(19) United States
(12) Patent Application Publication
(10) Pub. No.: US 2022/0281949 A1
(43) Pub. Date: Sep. 8, 2022

(54) MHC CLASS II MOLECULES AND METHODS OF USE THEREOF

(71) Applicant: University Health Network, Toronto (CA)

(72) Inventors: Naoto HIRANO, Toronto (CA); Kenji SUGATA, Toronto (CA); Tingxi GUO, Toronto (CA)

(73) Assignee: University Health Network, Toronto, ON (CA)

(21) Appl. No.: 17/631,824

(22) PCT Filed: Jul. 29, 2020
(86) PCT No.: PCT/IB2020/057174

§ 371 Date: Jan. 31, 2022

Related U.S. Application Data
(60) Provisional application No. 62/880,509, filed on Jul. 30, 2019.
(60) Provisional application No. 63/029,115, filed on May 22, 2020.

(51) Int. Cl. C07K 14/74; A61P 35/00

(52) U.S. Cl. CPC C07K 14/70539; A61P 35/00; A61K 38/00

(57) ABSTRACT

The present disclosure is directed to HLA class II molecules having a higher affinity for CD4 than naturally occurring HLA class II molecules. In certain aspects, the HLA class II molecule comprises a DR beta chain having (i) an amino acid other than leucine at a position corresponding to amino acid residue 114 of SEQ ID NO: 1, (ii) an amino acid other than valine at a position corresponding to amino acid residue 143 of SEQ ID NO: 1, (iii) or both (i) and (ii). Certain aspects of the present disclosure are directed to nucleic acid molecules encoding the HLA class II molecules, vectors comprising the nucleic acid molecule, cells comprising the same, and methods of use thereof.

### CROSS REFERENCE TO RELATED APPLICATIONS

This PCT application claims the priority benefit of U.S. Provisional Application Nos. 62/880,509, filed Jul. 30, 2019, and 63/029,115, filed May 22, 2020, each of which is incorporated herein by reference in its entirety.

### REFERENCE TO SEQUENCE LISTING SUBMITTED ELECTRONICALLY VIA EFS-WEB

The content of the electronically submitted sequence listing (Name: 4285-012PC02_SL_ST25.txt, Size: 63,907 bytes; and Date of Creation: Jul. 28, 2020) is incorporated herein by reference in its entirety.

### FIELD OF THE DISCLOSURE

The present disclosure provides major histocompatibility complex (MHC) class II molecules with increased affinity for CD4 and uses thereof.

### BACKGROUND OF THE DISCLOSURE

Immunotherapy has emerged as a critical tool in the battle against a variety of diseases, including cancer. T cell therapies are at the forefront of immunotherapeutic development, and adoptive transfer of antitumor T cells has been shown to induce clinical responses in cancer patients.

Directed T cell therapy using T cells expression T cell receptors (TCRs) specific for a target epitope expressed by tumor cells is a promising form of T cell therapy. Antigen presenting cells display peptide fragments associated with the major histocompatibility complex (MHC) on their surface to induce an immune response. It has been demonstrated that the improved presentation of endogenous peptides via class II is correlated with improved survival of cancer patients. However, the development of novel TCRs capable of specifically targeting MHC class II presented peptides is hindered by the low affinity of MHC class II proteins for CD4 expressed by T cells.

The present disclosure provides MHC class II proteins with increased affinity for CD4 and methods of using the same for the identification and development of novel MHC class II-specific TCRs.

### SUMMARY OF THE DISCLOSURE

Certain aspects of the present disclosure are directed to an HLA class II molecule comprising a DR beta chain, wherein the DR beta chain comprises an amino acid other than leucine at a position corresponding to amino acid residue 114 of SEQ ID NO: 1.

Certain aspects of the present disclosure are directed to an HLA class II molecule comprising a DR beta chain, wherein the DR beta chain comprises a substitution mutation at a position corresponding to amino acid residue 114 of SEQ ID NO: 1, wherein the substitution mutation is with an amino acid other than leucine.

In certain embodiments, the DR beta chain further comprises an amino acid other than valine at a position corresponding to amino acid residue 143 of SEQ ID NO: 1.

Certain aspects of the present disclosure are directed to an HLA class II molecule comprising a DR beta chain, wherein the DR beta chain comprises an amino acid other than valine at a position corresponding to amino acid residue 143 of SEQ ID NO: 1.

Certain aspects of the present disclosure are directed to an HLA class II molecule comprising a DR beta chain, wherein the DR beta chain comprises a substitution mutation at a position corresponding to amino acid residue 143 of SEQ ID NO: 1, wherein the substitution mutation is with an amino acid other than valine.

In certain embodiments, the DR beta chain further comprises an amino acid other than leucine at a position corresponding to amino acid residue 114 of SEQ ID NO: 1. In certain embodiments, the DR beta chain further comprises an amino acid other than asparagine at a position corresponding to amino acid residue 110 of SEQ ID NO: 1. In certain embodiments, the DR beta chain further comprises an amino acid other than isoleucine at a position corresponding to amino acid residue 116 of SEQ ID NO: 1. In certain embodiments, the DR beta chain further comprises an amino acid other than serine at a position corresponding to amino acid residue 118 of SEQ ID NO: 1. In certain embodiments, the DR beta chain further comprises an amino acid other than proline at a position corresponding to amino acid residue 146 of SEQ ID NO: 1.

In certain embodiments, the beta chain of the HLA class II molecule comprises a DR2, DR3, DR4, DR5, DR6, DR7, DR8, DR9, DR10, DR11, DR12, DR13, DR14, DR15, or DR16 allele. In certain embodiments, the beta chain of the MHC class II molecule comprises an HLA allele selected from the group consisting of DRB1*01, DRB1*03, DRB1*04, DRB1*07, DRB1*08, DRB1*09, DRB1*10, DRB1*11, DRB1*12, DRB1*13, DRB1*14, DRB1*15, and DRB1*16.

In certain embodiments, the HLA class II molecule further comprises an alpha chain. In certain embodiments, the alpha chain of the MHC class II molecule comprises an HLA-DRA1*01 allele.

In certain embodiments, the beta chain comprises (a) an amino acid other than leucine at a position corresponding to amino acid residue 114 of SEQ ID NO: 1; (b) an amino acid other than valine at a position corresponding to amino acid residue 143 of SEQ ID NO: 1; and (c) at least two of (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain embodiments, the beta chain comprises (c) at least three of (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain embodiments, the beta chain comprises (c) at least four of (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain embodiments, the beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, and (d) an amino acid other than threonine at a position corresponding to amino acid residue 157 of SEQ ID NO: 1.

In certain embodiments, the beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, (d) an amino acid other than lysine at a position corresponding to amino acid residue 139 of SEQ ID NO: 1, (e) an amino acid other than glycine at a position corresponding to amino acid residue 146 of SEQ ID NO: 1, (f) an amino acid other than threonine at a position corresponding to amino acid residue 157 of SEQ ID NO: 1, (g) an amino acid other than threonine at a position corresponding to amino acid residue 163 of SEQ ID NO: 1, and (h) an amino acid other than valine at a position corresponding to amino acid residue 164 of SEQ ID NO: 1.

In certain embodiments, the amino acid other than leucine at a position corresponding to amino acid residue 114 of SEQ ID NO: 1 comprises a hydrophobic side chain. In certain embodiments, the amino acid other than leucine at a position corresponding to amino acid residue 114 of SEQ ID NO: 1 is selected from the group consisting of an alanine, a valine, an isoleucine, a methionine, a phenylalanine, a tyrosine, and a tryptophan. In certain embodiments, the amino acid other than leucine at a position corresponding to amino acid residue 114 of SEQ ID NO: 1 is a tryptophan.

In certain embodiments, the amino acid other than valine at a position corresponding to amino acid residue 143 of SEQ ID NO: 1 comprises a hydrophobic side chain. In certain embodiments, the amino acid other than valine at a position corresponding to amino acid residue 143 of SEQ ID NO: 1 is selected from an alanine, an isoleucine, a leucine, a methionine, a phenylalanine, a tyrosine, and a tryptophan. In certain embodiments, the amino acid other than valine at a position corresponding to amino acid residue 143 of SEQ ID NO: 1 is a methionine.

In certain embodiments, the beta chain of the WIC class II molecule comprises an amino acid other than serine at a position corresponding to amino acid residue 118 of SEQ ID NO: 1. In certain embodiments, the amino acid other than serine at a position corresponding to amino acid residue 118 of SEQ ID NO: 1 is selected from an arginine, a histidine, and a lysine. In certain embodiments, the amino acid other than serine at a position corresponding to amino acid residue 118 of SEQ ID NO: 1 is a histidine.

In certain embodiments, the beta chain of the WIC class II molecule comprises an amino acid other than lysine at a position corresponding to amino acid residue 139 of SEQ ID NO: 1. In certain embodiments, the amino acid other than lysine at a position corresponding to amino acid residue 139 of SEQ ID NO: 1 is selected from a serine, a threonine, and a glutamine. In certain embodiments, the amino acid other than lysine at a position corresponding to amino acid residue 139 of SEQ ID NO: 1 is a threonine.

In certain embodiments, the beta chain of the WIC class II molecule comprises an amino acid other than glycine at a position corresponding to amino acid residue 146 of SEQ ID NO: 1. In certain embodiments, the amino acid other than glycine at a position corresponding to amino acid residue 146 of SEQ ID NO: 1 is selected from a serine, an asparagine, a threonine, and a glutamine. In certain embodiments, the amino acid other than glycine at a position corresponding to amino acid residue 146 of SEQ ID NO: 1 is a glutamine.

In certain embodiments, the beta chain of the WIC class II molecule comprises an amino acid other than threonine at a position corresponding to amino acid residue 157 of SEQ ID NO: 1. In certain embodiments, the amino acid other than threonine at a position corresponding to amino acid residue 157 of SEQ ID NO: 1 is selected from an alanine, a valine, an isoleucine, a leucine, a methionine, a phenylalanine, a tyrosine, and a tryptophan. In certain embodiments, the amino acid other than threonine at a position corresponding to amino acid residue 157 of SEQ ID NO: 1 is an isoleucine.

In certain embodiments, the beta chain of the WIC class II molecule comprises an amino acid other than threonine at a position corresponding to amino acid residue 163 of SEQ ID NO: 1. In certain embodiments, the amino acid other than threonine at a position corresponding to amino acid residue 163 of SEQ ID NO: 1 is selected from an alanine, a valine, an isoleucine, a leucine, a methionine, a phenylalanine, a tyrosine, and a tryptophan. In certain embodiments, the amino acid other than threonine at a position corresponding to amino acid residue 163 of SEQ ID NO: 1 is a methionine.

In certain embodiments, the beta chain of the WIC class II molecule comprises an amino acid other than valine at a position corresponding to amino acid residue 164 of SEQ ID NO: 1. In certain embodiments, the amino acid other than valine at a position corresponding to amino acid residue 164 of SEQ ID NO: 1 is selected from a serine, an asparagine, a threonine, and a glutamine. In certain embodiments, the amino acid other than valine at a position corresponding to amino acid residue 164 of SEQ ID NO: 1 is a threonine.

In certain embodiments, the beta chain comprises (a) a tryptophan at a position corresponding to amino acid residue 114 of SEQ ID NO: 1, (b) a methionine at a position corresponding to amino acid residue 143 of SEQ ID NO: 1, (c) a histidine at a position corresponding to amino acid residue 118 of SEQ ID NO: 1, and (d) an isoleucine at a position corresponding to amino acid residue 157 of SEQ ID NO: 1.

In certain embodiments, the DR beta chain comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 3, 4, and 5.

In certain embodiments, the beta chain comprises (a) a tryptophan at a position corresponding to amino acid residue 114 of SEQ ID NO: 1; (b) a methionine at a position corresponding to amino acid residue 143 of SEQ ID NO: 1; and (c) at least two of (i) a histidine at a position corresponding to amino acid residue 118 of SEQ ID NO: 1, (ii) a threonine at a position corresponding to amino acid residue 139 of SEQ ID NO: 1, (iii) a glutamine at a position corresponding to amino acid residue 146 of SEQ ID NO: 1, (iv) an isoleucine at a position corresponding to amino acid residue 157 of SEQ ID NO: 1, (v) a methionine at a position corresponding to amino acid residue 163 of SEQ ID NO: 1, and (vi) a threonine at a position corresponding to amino acid residue 164 of SEQ ID NO: 1.

In certain embodiments, the beta chain comprises (a) a tryptophan at a position corresponding to amino acid residue 114 of SEQ ID NO: 1; (b) a methionine at a position corresponding to amino acid residue 143 of SEQ ID NO: 1; and (c) at least three of (i) a histidine at a position corresponding to amino acid residue 118 of SEQ ID NO: 1, (ii) a threonine at a position corresponding to amino acid residue 139 of SEQ ID NO: 1, (iii) a glutamine at a position corresponding to amino acid residue 146 of SEQ ID NO: 1, (iv) an isoleucine at a position corresponding to amino acid residue 157 of SEQ ID NO: 1, (v) a methionine at a position corresponding to amino acid residue 163 of SEQ ID NO: 1, and (vi) a threonine at a position corresponding to amino acid residue 164 of SEQ ID NO: 1.

In certain embodiments, the beta chain comprises (a) a tryptophan at a position corresponding to amino acid residue 114 of SEQ ID NO: 1; (b) a methionine at a position corresponding to amino acid residue 143 of SEQ ID NO: 1; and (c) at least four of (i) a histidine at a position corresponding to amino acid residue 118 of SEQ ID NO: 1, (ii) a threonine at a position corresponding to amino acid residue 139 of SEQ ID NO: 1, (iii) a glutamine at a position corresponding to amino acid residue 146 of SEQ ID NO: 1, (iv) an isoleucine at a position corresponding to amino acid residue 157 of SEQ ID NO: 1, (v) a methionine at a position corresponding to amino acid residue 163 of SEQ ID NO: 1, and (vi) a threonine at a position corresponding to amino acid residue 164 of SEQ ID NO: 1.

In certain embodiments, the beta chain comprises (a) a tryptophan at a position corresponding to amino acid residue 114 of SEQ ID NO: 1, (b) a methionine at a position corresponding to amino acid residue 143 of SEQ ID NO: 1, (c) a histidine at a position corresponding to amino acid residue 118 of SEQ ID NO: 1, (d) a threonine at a position corresponding to amino acid residue 139 of SEQ ID NO: 1, (e) a glutamine at a position corresponding to amino acid residue 146 of SEQ ID NO: 1, (f) an isoleucine at a position corresponding to amino acid residue 157 of SEQ ID NO: 1, (g) a methionine at a position corresponding to amino acid residue 163 of SEQ ID NO: 1, and (h) a threonine at a position corresponding to amino acid residue 164 of SEQ ID NO: 1.

In certain embodiments, the DR beta chain comprises the amino acid sequence set forth in SEQ ID NO: 3. In certain embodiments, the DR beta chain comprises the amino acid sequence set forth in SEQ ID NO: 4.

In certain embodiments, the beta chain of the HLA class II molecule comprises a DR1, DR3, DR4, DR7, DR8, DR9, DR10, DR11, DR12, DR13, DR14, DR15, or DR16 allele. In certain embodiments, the beta chain of the MEW class II molecule comprises an HLA-DRB1*01, HLA-DRB1*03, HLA-DRB1*04, HLA-DRB1*06, HLA-DRB1*07, HLA-DRB1*08, HLA-DRB1*09, HLA-DRB1*10, HLA-DRB1*11, HLA-DRB1*12, HLA-DRB1*13, HLA-DRB1*14, HLA-DRB1*15, or HLA-DRB1*16 allele. In certain embodiments, the alpha chain of the MHC class II molecule comprises an HLA-DRA1*01 allele.

In certain embodiments, the DR alpha chain comprises an amino acid sequence having at least about 80%, at least about 85%, at least about 90%, at least about 95%, at least about 96%, at least about 97%, at least about 98%, or at least about 99% sequence identity to SEQ ID NO: 6 or 8. In certain embodiments, the DR alpha chain comprises the amino acid sequence set forth in SEQ ID NO: 6 or 8.

In certain embodiments, the DR beta chain has an increased affinity for a CD4 protein as compared to a reference HLA class II molecule, wherein the reference HLA class II molecule comprises a DR beta chain comprising (i) a leucine at a position corresponding to amino acid residue 114 of SEQ ID NO: 1 and/or (ii) a valine at a position corresponding to amino acid residue 143 of SEQ ID NO: 1.

In certain embodiments, the increased affinity is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, or at least about 1000.

In certain embodiments, the DR beta chain is bound to a membrane of a cell. In certain embodiments, the DR beta chain is not bound to a membrane of a cell. In certain embodiments, the DR beta chain comprises an extracellular domain of a full length DR alpha chain. In certain embodiments, the DR beta chain does not comprise a transmembrane domain of a full length DR beta chain.

In certain embodiments, the DR alpha chain is bound to a membrane of a cell. In certain embodiments, the DR alpha chain is not bound to a membrane of a cell. In certain embodiments, the DR alpha chain comprises an extracellular domain of a full length DR alpha chain. In certain embodiments, the DR alpha chain does not comprise a transmembrane domain of a full length DR alpha chain.

In certain embodiments, the DR beta chain is linked to or associated with an inert particle. In certain embodiments, the inert particle is a bead. In certain embodiments, the inert particle is a nanoparticle. In certain embodiments, the nanoparticle is selected from a pegylated iron oxide, chitosan, dextrane, gelatin, alginate, liposome, starch, branched polymer, carbon-based carrier, polylactic acid, poly(cyano)acrylate, polyethyleinemine, block copolymer, ply caprolactone, SPIONS, USPIONS, Cd/Zn-selenide, or silica nanoparticle. In certain embodiments, the nanoparticle is a pegylated iron oxide nanoparticle.

In certain embodiments, the DR beta chain comprises a signal peptide. In certain embodiments, the DR alpha chain comprises a signal peptide. In certain embodiments, the signal peptide comprises the amino acid sequence set forth in SEQ ID NO: 9.

Certain aspects of the present disclosure are directed to a nucleic acid molecule encoding a DR beta chain disclosed herein. In certain embodiments, the nucleic acid molecule further encodes a DR alpha chain disclosed herein. In certain embodiments, the nucleic acid molecule comprises a nucleotide sequence having at least about 70%, at least about 75%, at least about 80%, at least about 85%, at least about 90%, at least about 95%, at least about 96%, at least about 97%, at least about 98%, or at least about 99% sequence identity to SEQ ID NO: 2.

Certain aspects of the present disclosure are directed to a vector comprising a nucleic acid molecule disclosed herein.

Certain aspects of the present disclosure are directed to a cell comprising an HLA class II molecule disclosed herein, a nucleic acid molecule disclosed herein, or a vector disclosed herein. In certain embodiments, the cell is a mammalian cell or an insect cell. In certain embodiments, the cell is selected from a K562 cell, T2, HEK293, HEK293T, A375, SK-MEL-28, Me275, COS, a fibroblast cell, a tumor cell, or any combination thereof. In certain embodiments, the cell lacks endogenous MHC class II DR beta chain expression. In certain embodiments, the cell lacks endogenous MHC class II DR alpha chain expression.

Certain aspects of the present disclosure are directed to a method of identifying a T cell receptor capable of binding an epitope in an MHC class II complex, comprising pulsing a cell disclosed herein with one or more peptide comprising the epitope, and stimulating one or more CD4⁺ T cell with the APC.

Certain aspects of the present disclosure are directed to a method of treating a disease or condition in a subject in need thereof, comprising administering to the subject an MHC class II molecule disclosed herein. In certain embodiments, the disease or condition is cancer or an infection.

In certain embodiments, the cancer is selected from the group consisting of melanoma, bone cancer, pancreatic cancer, skin cancer, cancer of the head or neck, uterine cancer, ovarian cancer, rectal cancer, stomach cancer, uterine cancer, lung cancer, Hodgkin's Disease, non-Hodgkin's lymphoma (NHL), cancer of the esophagus, cancer of the small intestine, cancer of the urethra, chronic or acute leukemia, acute myeloid leukemia, chronic myeloid leukemia, acute lymphoblastic leukemia (ALL) (including non T cell ALL), chronic lymphocytic leukemia (CLL), cancer of the bladder, cancer of the kidney or ureter, carcinoma of the renal pelvis, glioma, squamous cell cancer, and combinations of said cancers.

In certain embodiments, the cancer is relapsed or refractory. In certain embodiments, the cancer is locally advanced. In certain embodiments, the cancer is advanced. In certain embodiments, the cancer is metastatic.

In some aspects, the HLA class II molecule binds CD4 with a K_{D} of less than about 100 μM. In some aspects, the HLA class II molecule binds CD4 with a K_{D} of less than about 20 μM. In some aspects, the HLA class II molecule binds CD4 with a K_{D} of about 14 μM or less.

Certain aspects of the disclosure are directed to a complex comprising an HLA class II molecule disclosed herein and a peptide, wherein the peptide is selected from NY-ESO1₉₁₋₁₁₀, Bet V 1₁₄₂₋₁₅₃, HIV Gag₂₉₃₋₃₁₂, HA₃₀₆₋₃₁₈, Flu-HA₅₋₂₄, Flu-HA₁₁₇₋₁₃₆, Flu-HA₂₃₂₋₂₅₁, Flu-HA₂₆₈₋₂₈₇, Flu-HA₃₀₆₋₃₁₈, HSD17B12₂₂₅₋₂₄₄, LY6K₉₉₋₁₁₈, and any combination thereof.

### BRIEF DESCRIPTION OF THE DRAWINGS

FIGS. 1A-1F provide data illustrating the enhanced CD4 binding ability of modified DR molecules. FIG. 1A is a table comparing the amino acid sequences of DPB1*04:01, DRB1*01:01, and DRB1*01:0^{L114W/V143M+6reps}, with mutated amino acids underlined. FIGS. 1B and 1C are graphical representations of data of class II-deficient K562 cells stably transduced with wild-type DR1 (DRA1*01:01/DRB1*01:01), DR1^{L114W/V143M}, DR1^{L114W/V143M+6reps}, wild-type DP4, or DP4^{L112W/V141M} and stained with sCD4. FIGS. 1D-1E show the CD4 binding ability of a series of K562 derivatives individually expressing DR1^{L114W/V143M+6reps} mutants with a single amino acid reversal at one of the six positions (FIG. 1D), similarly stained with sCD4 and DRB1^{L114W/V143M+2reps}, which carries S118H and T157I along with L114W/V143M (FIG. 1E). FIG. 1F is a table listing the amino acid sequences of DPB1*04:01 and DRB1 alleles of DR3, DR4, DR7, DR10, DR11, and DR13 were compared along with those of DRB1^{L114W/V143M+6reps} and DRB1^{L114W/V143M+2reps}, with mutated amino acids underlined. FIGS. 1G-1L are graphical representations of data showing that the L114W/V143M+2reps mutations enhanced the binding of DR3, DR4, DR7, DR10, DR11, and DR13 to CD4 better than the L114W/V143M+6reps mutations. At least 2 independent experiments were performed. *, P<0.05 by Student's t-test. Bars and error bars represent the mean±SD of results in triplicate experiments. FIGS. 1M-1N are biolayer interferometry sensorgrams showing the interaction of biotinylated HLA-DR1 (ligand) with soluble CD4 (analyte) over a range of concentrations. Binding experiments for wild-type DR1 (FIG. 1M) and DR1^{L114W/V143M+2reps} (FIG. 1N) were performed in parallel, and binding was not detected for wild-type DR1 (FIG. 1M). FIG. 1O is a graph showing the affinity between DR1^{L114W/V143M+2reps} and CD4 as quantified by steady-state analysis. All data are representative of two independent experiments.

FIGS. 2A-2D are graphical representations illustrating that affinity-matured DR dimers detected cognate TCRs are expressed in human primary CD4+ T cells. DR1-restricted TCRs (HA1.7 and SB95) (FIG. 2A), DR7-restricted TCR (SD334) (FIG. 2B) and DR11-restricted TCR (F24) (FIG. 2C) were reconstituted in primary human T cells and stained by respective DR^{L114W/V143M+2reps} dimers. DR11-restricted F24-transduced CD4⁺ T cells were stained with the DR11^{L114W/V143M+2reps} dimer and an anti-Vβ22 mAb (FIG. 2D). Note that F24 expresses Vβ22. At least 2 independent experiments were performed.

FIGS. 3A-3D are drawings of model structures of HLA-DR1^{L114W/V143M+2reps} and the human CD4 complex. FIG. 3A provides an overview ribbon model of the ternary complex model structure of DRA1*01:01, DRB1*01:01, and CD4, as indicated. FIGS. 3B-3D provide close-up views of four mutated residues: L114W and V143M (FIG. 3B), S118H (FIG. 3C) and T157I (FIG. 3D) in wild-type DR1 (left) and mutated DR1^{L114W/V143M+2reps} (right), as illustrated using ball-and-stick representation.

FIGS. 4A-4II are graphical representations of histograms illustrating the comparable expression levels of HLA class II genes. HLA-DR and their derivatives were reconstituted in K562 cells and stained with anti-HLA class II monoclonal antibodies. The surface expression of all DR alleles was detected using the anti-HLA class II monoclonal antibody clone 9-49(I3). Open histograms represent the isotype control staining.

FIGS. 5A-5L are graphical representations showing influenza virus hemagglutinin-specific peripheral CD4⁺ T cells subjected to ex vivo staining with DR1^{L114W/V143M+2reps} dimers. Memory CD⁴⁺ T cells were purified from two DR1⁺ donors (No. 07 (FIGS. 5A-5F) and No. 08 (FIGS. 5G-5L)) and stained with DR1^{L114W/V143M+2reps} dimers specific to Flu-HA₅₋₂₄ (FIGS. 5B and 5H), Flu-HA₁₁₇₋₁₃₆ (FIGS. 5C and 5I), Flu-HA₂₃₂₋₂₅₁ (FIGS. 5D and 5J), Flu-HA₂₆₈₋₂₈₇ (FIGS. 5E and 5K), and Flu-HA₃₀₆₋₃₁₈ (FIGS. 5F and 5L) influenza virus hemagglutinin (Flu-HA) peptides without in vitro stimulation. The CLIP peptide was used as a negative control (FIGS. 5A and 5G).

FIGS. 6A-6X are graphical representations showing DR1^{L114W/V143M+6reps} and DR1^{L114W/V143M+2reps} dimers robustly stained HA1.7-transduced CD4⁺ T cells. HA1.7 was reconstituted in primary CD4⁺ T cells, which were then stained with wild-type DR1 (FIGS. 6I and 6M), DR1^{L114W/V143M} (FIGS. 6J and 6N), DR1^{L114W/V143M+6reps} (FIGS. 6K and 6O), and DR1^{L114W/V143M+2reps} (FIGS. 6L and 6P) dimers without a transduced TCR (FIGS. 6I-6L) and transduced with an HA1.7 TCR (FIGS. 6M-6P), with CLIP dimers used as negative controls (FIGS. 6A-6H). In addition, HA1.7 was reconstituted in primary CD4⁺ T cells, which were then stained with DR1^{L114W/V143M+2reps} dimer (FIGS. 6Q-6T) or a wild-type DR1 dextramer (FIGS. 6U-6X) specific to Flu-HA₃₀₆₋₃₁₈.

FIGS. 7A-7O are graphical representations showing data for cloning of DR1-restricted TCRs using affinity matured dimer. Primary CD4⁺ T cells were purified from two DR1⁺ melanoma patients and stimulated with irradiated HSD17B12₂₂₅₋₂₄₄-pulsed (FIG. 7B) and LY6K₉₉₋₁₁₈-pulsed (FIG. 7D) aAPCs expressing DR1. Two weeks later, stimulated CD4⁺ T cells were stained with cognate DR1^{L114W/V143M+2reps} dimers (FIGS. 7A-7D). The DR1-restricted TCRs were reconstituted in primary CD4⁺ T cells, and stained by the respective dimer (FIGS. 7E-7M). Primary CD4⁺ T cells expressing the DR1-restricted DR1-07-HSD17B12₂₂₅₋₂₄₄ (FIG. 7N) and DR1-08-LY6K₉₉₋₁₁₈ (FIG. 7O) TCRs were stimulated by DR1-K562 cells pulsed with HSD17B12₂₂₅₋₂₄₄ (FIG. 7N) and LY6K₉₉₋₁₁₈ (FIG. 7O) peptides, respectively, in IL-2 ELISPOT assays.

### DETAILED DESCRIPTION OF THE DISCLOSURE

The present disclosure is directed to MHC class II molecules with increased affinity for CD4. In some aspects, the present disclosure is directed to MEW class II molecules comprising an HLA-DR (DR) beta chain, wherein the DR beta chain has increased affinity for CD4.

The present disclosure is further directed to MHC class II molecules comprising a DR beta chain, wherein the DR beta chain comprises an amino acid other than leucine at a position corresponding to amino acid residue 114 of SEQ ID NO: 1. In some aspects, the DR beta chain further comprises an amino acid other than valine at a position corresponding to amino acid residue 143 of SEQ ID NO: 1.

The present disclosure is further directed to MHC class II molecules comprising a DR beta chain, wherein the DR beta chain comprises an amino acid other than valine at a position corresponding to amino acid residue 143 of SEQ ID NO: 1. In some aspects, the DR beta chain further comprises an amino acid other than leucine at a position corresponding to amino acid residue 114 of SEQ ID NO: 1.

In some aspects, the DR beta chain further comprises at least two of: (i) a histidine at a position corresponding to amino acid residue 118 of SEQ ID NO: 1, (ii) a threonine at a position corresponding to amino acid residue 139 of SEQ ID NO: 1, (iii) a glutamine at a position corresponding to amino acid residue 146 of SEQ ID NO: 1, (iv) an isoleucine at a position corresponding to amino acid residue 157 of SEQ ID NO: 1, (v) a methionine at a position corresponding to amino acid residue 163 of SEQ ID NO: 1, and (vi) a threonine at a position corresponding to amino acid residue 164 of SEQ ID NO: 1.

In some aspects, the DR beta chain comprises (a) an amino acid other than valine at a position corresponding to amino acid residue 143 of SEQ ID NO: 1; (b) an amino acid other than leucine at a position corresponding to amino acid residue 114 of SEQ ID NO: 1; (c) an amino acid other than serine at a position corresponding to amino acid residue 118 of SEQ ID NO: 1; and (d) an amino acid other than threonine at a position corresponding to amino acid residue 157 of SEQ ID NO: 1.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “HLA,” as used herein, refers to the human leukocyte antigen. HLA genes encode the major histocompatibility complex (MHC) proteins in humans. MHC proteins are expressed on the surface of cells, and are involved in activation of the immune response. HLA class II genes encode MHC class II proteins which are expressed on the surface of professional antigen presenting cells (APCs). Non-limiting examples of professional APCs include monocytes, macrophages, dendritic cells (DCs), and B lymphocytes. Some endothelial and epithelial cells can also express MHC class II molecules after inflammatory signals are activated. Humans lacking functional MHC class II molecules are extremely susceptible to an array of infectious diseases and typically die at a young age.

As used herein, an “HLA class II molecule” or “MHC class II molecule” refers to a protein product of a wild-type or variant HLA class II gene encoding an MHC class II molecule. Accordingly, “HLA class II molecule” and “MHC class II molecule” are used interchangeably herein. A typical MHC Class II molecule comprises two protein chains: an alpha chain and a beta chain. In general, naturally occurring alpha chains and beta chains each comprise a transmembrane domain, which anchors the alpha/beta chain to the cell surface, and an extracellular domain, which carries the antigen and interacts with a TCR and/or CD4 expressed on a T cell.

Both the MHC Class II alpha and beta chains are encoded by the HLA gene complex. The HLA complex is located within the 6p21.3 region on the short arm of human chromosome 6 and contains more than 220 genes of diverse function. The HLA gene complex is highly variant, with over 20,000 HLA alleles and related alleles, including over 250 MHC class II alpha chain alleles and 5,000 MHC class II beta chain alleles, known in the art, encoding thousands of MHC class II proteins (see, e.g., hla.alleles.org, last visited May 20, 2019, which is incorporated by reference herein in its entirety). For example, one such HLA-DP allele, DP4 is the most frequently found allele in many ethnic groups. Each alpha chain and beta chain is typically expressed as a proprotein, which further comprises a signal peptide that is cleaved off. Any number of naturally occurring signal peptides can be used to facilitate expression and localization of the alpha chains and beta chains disclosed herein. One such example is SEQ ID NO: 9.

Three loci in the HLA complex encode MHC Class II proteins: HLA-DP, HLA-DQ, and HLA-DR. HLA-DO and HLA-DM encode proteins that associate with the MHC class II molecule and support its configuration and function. Representative HLA-DR sequences are provided in Table 1.

TABLE 1
DR Beta chain and alpha chain amino acid and nucleotide sequences.
Beta Chain
DRB1*01:01 Extracellular Domain (SEQ ID NO: 1)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNLLVCSVSGFYPGSIEVRWFRNGQEEKAGVVSTGLI
QNGDWTFQTLVMLETVPRSGEVYTCQVEHPSVTSPLTVEWRARSESAQSK
DRB1*01:01 Extracellular Domain (SEQ ID NO: 2)
GGGGACACCCGACCACGTTTCTTGTGGCAGCTTAAGTTTGAATGTCATTTCTTCAATGGGACGGAGCGGGTGCG
GTTGCTGGAAAGATGCATCTATAACCAAGAGGAGTCCGTGCGCTTCGACAGCGACGTGGGGGAGTACCGGGCGG
TGACGGAGCTGGGGCGGCCTGATGCCGAGTACTGGAACAGCCAGAAGGACCTCCTGGAGCAGAGGCGGGCCGCG
GTGGACACCTACTGCAGACACAACTACGGGGTTGGTGAGAGCTTCACAGTGCAGCGGCGAGTTGAGCCTAAGGT
GACTGTGTATCCTTCAAAGACCCAGCCCCTGCAGCACCACAACCTCCTGGTCTGCTCTGTGAGTGGTTTCTATC
CAGGCAGCATTGAAGTCAGGTGGTTCCGGAACGGCCAGGAAGAGAAGGCTGGGGTGGTGTCCACAGGCCTGATC
CAGAATGGAGATTGGACCTTCCAGACCCTGGTGATGCTGGAAACAGTTCCTCGGAGTGGAGAGGTTTACACCTG
CCAAGTGGAGCACCCAAGTGTGACGAGCCCTCTCACAGTGGAATGGAGAGCACGGTCTGAATCTGCACAGAGCA
AG
DRB1*01:01 L114W/V143M Extracellular Domain (SEQ ID NO: 11)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCSVSGFYPGSIEVRWFRNGQEEKAGVMSTGLI
QNGDWTFQTLVMLETVPRSGEVYTCQVEHPSVTSPLTVEWRARSESAQSK
DRB1*01:01 L114W/V143M + 6reps Extracellular Domain (SEQ ID NO: 12)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCHVSGFYPGSIEVRWERNGQEETAGVMSTNLI
QNGDWTFQILVMLEMTPRSGEVYTCQVEHPSVTSPLTVEWRARSESAQSK
DRB1*01:01 L114W/V143M/S118H/T157I Extracellular Domain (SEQ ID NO: 3)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCHVSGFYPGSIEVRWERNGQEEKAGVMSTGLI
QNGDWTFQILVMLETVPRSGEVYTCQVEHPSVTSPLTVEWRARSESAQSK
Signal Peptide; DRB1*01:01 L114W/V143M/S118H/T157I Extracellular Domain;
Gly/Ser Linker; Zip Sequences and His tag sequences) (SEQ ID NO: 4)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCHVSGFYPGSIEVRW
FRNGQEEKAGVMSTGLIQNGDWTFQILVMLETVPRSGEVYTCQVEHPSVTSPLTVEWRARSESAQSKGGGGSLE
IEAAFLERENTALETRVAELRQRVQRLRNRVSQYRTRYGPLGGGK
Full-length wild-type DRB1*01:01 (SEQ ID NO: 5)
MVCLKLPGGSCMTALTVTLMVLSSPLALAGDTRPRFLWQLKFECHFFNGTERVRLLERCIYNQEESVRFDSDVG
EYRAVTELGRPDAEYWNSQKDLLEQRRAAVDTYCRHNYGVGESFTVQRRVEPKVTVYPSKTQPLQHHNLLVCSV
SGFYPGSIEVRWFRNGQEEKAGVVSTGLIQNGDWTFQTLVMLETVPRSGEVYTCQVEHPSVTSPLTVEWRARSE
SAQSKMLSGVGGFVLGLLFLGAGLFIYFRNQKGHSGLQPTGFLS
Signal Peptide; DRB1*01:01 Extracellular Domain; and Gly/Ser Linker, Zip
Sequences, and biotinylation sequences) (SEQ ID NO: 15)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNLLVCSVSGFYPGSIEVRW
FRNGQEEKAGVVSTGLIQNGDWTFQTLVMLETVPRSGEVYTCQVEHPSVTSPLTVEWRARSESAQSKGGGGSLE
IEAAFLERENTALETRVAELRQRVQRLRNRVSQYRTRYGPLGGGKGSGLNDIFEAQKIEWHE
Signal Peptide; DRB1*01:01 Extracellular Domain; and Gly/Ser Linker, Zip
Sequences, and biotinylation sequences) (SEQ ID NO: 16)
ATGATGCGGCCCATCGTGCTGGTGCTGCTGTTCGCCACATCTGCCCTGGCCGGGGACACCCGACCACGTTTCTT
GTGGCAGCTTAAGTTTGAATGTCATTTCTTCAATGGGACGGAGCGGGTGCGGTTGCTGGAAAGATGCATCTATA
ACCAAGAGGAGTCCGTGCGCTTCGACAGCGACGTGGGGGAGTACCGGGCGGTGACGGAGCTGGGGCGGCCTGAT
GCCGAGTACTGGAACAGCCAGAAGGACCTCCTGGAGCAGAGGCGGGCCGCGGTGGACACCTACTGCAGACACAA
CTACGGGGTTGGTGAGAGCTTCACAGTGCAGCGGCGAGTTGAGCCTAAGGTGACTGTGTATCCTTCAAAGACCC
AGCCCCTGCAGCACCACAACCTCCTGGTCTGCTCTGTGAGTGGTTTCTATCCAGGCAGCATTGAAGTCAGGTGG
TTCCGGAACGGCCAGGAAGAGAAGGCTGGGGTGGTGTCCACAGGCCTGATCCAGAATGGAGATTGGACCTTCCA
GACCCTGGTGATGCTGGAAACAGTTCCTCGGAGTGGAGAGGTTTACACCTGCCAAGTGGAGCACCCAAGTGTGA
CGAGCCCTCTCACAGTGGAATGGAGAGCACGGTCTGAATCTGCACAGAGCAAGGGCGGCGGAGGCAGCCTGGAA
ATCGAGGCCGCCTTCCTGGAAAGAGAGAACACCGCCCTGGAAACCCGGGTGGCCGAGCTGAGACAGAGAGTGCA
GAGACTGCGGAACCGGGTGTCCCAGTACCGGACCAGATATGGCCCTCTGGGAGGCGGCAAAGGGTCCGGCTTGA
ACGACATTTTTGAGGCCCAGAAGATAGAGTGGCACGAGTGA
Signal Peptide; DRB1*01:01 L114W/V143M Extracellular Domain; Gly/Ser
Linker; Zip Sequences and His tag sequences) (SEQ ID NO: 14)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCSVSGFYPGSIEVRW
FRNGQEEKAGVMSTGLIQNGDWTFQTLVMLETVPRSGEVYTCQVEHPSVTSPLTVEWRARSESAQSKGGGGSLE
IEAAFLERENTALETRVAELRQRVQRLRNRVSQYRTRYGPLGGGK
Signal Peptide; DRB1*01:01 [symbol] [symbol] ( [symbol] ) Extracellular
Domain; Gly/Ser Linker, Zip Sequences, and biotinylation sequences) (SEQ
ID NO: 17)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHN [symbol] LVC [symbol] VSGFYPGSIEVRW
FRNGQEEKAGV [symbol] STGLIQNGDWTFQ [symbol] LVMLETVPRSGEVYTCQVEHPSVTSPLTVEWRARSESAQSKGGGGSLE
IEAAFLERENTALETRVAELRQRVQRLRNRVSQYRTRYGPLGGGKGSGLNDIFEAQKIEWHE
Signal Peptide; DRB1*01:01 [symbol] [symbol] ( [symbol] ) Extracellular
Domain; Gly/Ser Linker, Zip Sequences, and biotinylation sequences) (SEQ
ID NO: 18)
ATGATGCGGCCCATCGTGCTGGTGCTGCTGTTCGCCACATCTGCCCTGGCCGGGGACACCCGACCACGTTTCTT
GTGGCAGCTTAAGTTTGAATGTCATTTCTTCAATGGGACGGAGCGGGTGCGGTTGCTGGAAAGATGCATCTATA
ACCAAGAGGAGTCCGTGCGCTTCGACAGCGACGTGGGGGAGTACCGGGCGGTGACGGAGCTGGGGCGGCCTGAT
GCCGAGTACTGGAACAGCCAGAAGGACCTCCTGGAGCAGAGGCGGGCCGCGGTGGACACCTACTGCAGACACAA
CTACGGGGTTGGTGAGAGCTTCACAGTGCAGCGGCGAGTTGAGCCTAAGGTGACTGTGTATCCTTCAAAGACCC
AGCCCCTGCAGCACCACAAC [symbol] CTGGTCTGC [symbol] GTGAGTGGTTTCTATCCAGGCAGCATTGAAGTCAGGTGG
TTCCGGAACGGCCAGGAAGAGAAGGCTGGGGTG [symbol] TCCACAGGCCTGATCCAGAATGGAGATTGGACCTTCCA
G [symbol] CTGGTGATGCTGGAAACAGTTCCTCGGAGTGGAGAGGTTTACACCTGCCAAGTGGAGCACCCAAGTGTGA
CGAGCCCTCTCACAGTGGAATGGAGAGCACGGTCTGAATCTGCACAGAGCAAGGGCGGCGGAGGCAGCCTGGAA
ATCGAGGCCGCCTTCCTGGAAAGAGAGAACACCGCCCTGGAAACCCGGGTGGCCGAGCTGAGACAGAGAGTGCA
GAGACTGCGGAACCGGGTGTCCCAGTACCGGACCAGATATGGCCCTCTGGGAGGCGGCAAAGGGTCCGGCTTGA
ACGACATTTTTGAGGCCCAGAAGATAGAGTGGCACGAGTGA
Signal Peptide; DRB1*01:01 L114W/V143M + 6reps Extracellular Domain;
Gly/Ser Linker; Zip Sequences and His tag sequences) (SEQ ID NO: 17)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCHVSGFYPGSIEVRW
FRNGQEETAGVMSTNLIQNGDWTFQILVMLEMTPRSGEVYTCQVEHPSVTSPLTVEWRARSESAQSKGGGGSLE
IEAAFLERENTALETRVAELRQRVQRLRNRVSQYRTRYGPLGGGK
Alpha Chain
DRA1*01: 01 Extracellular Domain (SEQ ID NO: 6)
IKEEHVIIQAEFYLNPDQSGEFMFDFDGDEIFHVDMAKKETVWRLEEFGRFASFEAQGALANIAVDKANLEIMT
KRSNYTPITNVPPEVTVLTNSPVELREPNVLICFIDKFTPPVVNVTWLRNGKPVTTGVSETVFLPREDHLFRKF
HYLPFLPSTEDVYDCRVEHWGLDEPLLKHWEFDAPSPLPETTEN
DRA1*01:01 Extracellular Domain (SEQ ID NO: 7)
ATCAAAGAAGAACATGTGATCATCCAGGCCGAGTTCTATCTGAATCCTGACCAATCAGGCGAGTTTATGTTTGA
CTTTGATGGTGATGAGATTTTCCATGTGGATATGGCAAAGAAGGAGACGGTCTGGCGGCTTGAAGAATTTGGAC
GATTTGCCAGCTTTGAGGCTCAAGGTGCATTGGCCAACATAGCTGTGGACAAAGCCAACCTGGAAATCATGACA
AAGCGCTCCAACTATACTCCGATCACCAATGTACCTCCAGAGGTAACTGTGCTCACAAACAGCCCTGTGGAACT
GAGAGAGCCCAACGTCCTCATCTGTTTCATAGACAAGTTCACCCCACCAGTGGTCAATGTCACGTGGCTTCGAA
ATGGAAAACCTGTCACCACAGGAGTGTCAGAGACAGTCTTCCTGCCCAGGGAAGACCACCTTTTCCGCAAGTTC
CACTATCTCCCCTTCCTGCCCTCAACTGAGGACGTTTACGACTGCAGGGTGGAGCACTGGGGCTTGGATGAGCC
TCTTCTCAAGCACTGGGAGTTTGATGCTCCAAGCCCTCTCCCAGAGACTACAGAGAAC
Signal Peptide; DRA1*01:01 Extracellular Domain; Gly/Ser Linker, Zip
Sequences and His tag sequences) (SEQ ID NO: 8)
MMRPIVLVLLFATSALAIKEEHVIIQAEFYLNPDQSGEFMFDFDGDEIFHVDMAKKETVWRLEEFGRFASFEAQ
GALANIAVDKANLEIMTKRSNYTPITNVPPEVTVLTNSPVELREPNVLICFIDKFTPPVVNVTWLRNGKPVTTG
VSETVFLPREDHLFRKFHYLPFLPSTEDVYDCRVEHWGLDEPLLKHWEFDAPSPLPETTENGGGGSLEIRAAFL
RQRNTALRTEVAELEQEVQRLENEVSQYETRYGPLGGGKGSHHHHHH
Signal Peptide; DRA1*01:01 Extracellular Domain; Gly/Ser Linker, Zip
Sequences and His tag sequences (10x) (SEQ ID NO: 19)
MMRPIVLVLLFATSALAIKEEHVIIQAEFYLNPDQSGEFMFDFDGDEIFHVDMAKKETVWRLEEFGRFASFEAQ
GALANIAVDKANLEIMTKRSNYTPITNVPPEVTVLTNSPVELREPNVLICFIDKFTPPVVNVTWLRNGKPVTTG
VSETVFLPREDHLFRKFHYLPFLPSTEDVYDCRVEHWGLDEPLLKHWEFDAPSPLPETTENGGGGSLEIRAAFL
RQRNTALRTEVAELEQEVQRLENEVSQYETRYGPLGGGKGSHHHHHHHHHH
Signal Peptide; DRA1*01:01 Extracellular Domain; Gly/Ser Linker, Zip
Sequences and His tag sequences (10x) (SEQ ID NO: 20)
ATGATGCGGCCCATCGTGCTGGTGCTGCTGTTCGCCACATCTGCCCTGGCCATCAAAGAAGAACATGTGATCAT
CCAGGCCGAGTTCTATCTGAATCCTGACCAATCAGGCGAGTTTATGTTTGACTTTGATGGTGATGAGATTTTCC
ATGTGGATATGGCAAAGAAGGAGACGGTCTGGCGGCTTGAAGAATTTGGACGATTTGCCAGCTTTGAGGCTCAA
GGTGCATTGGCCAACATAGCTGTGGACAAAGCCAACCTGGAAATCATGACAAAGCGCTCCAACTATACTCCGAT
CACCAATGTACCTCCAGAGGTAACTGTGCTCACGAACAGCCCTGTGGAACTGAGAGAGCCCAACGTCCTCATCT
GTTTCATCGACAAGTTCACCCCACCAGTGGTCAATGTCACGTGGCTTCGAAATGGAAAACCTGTCACCACAGGA
GTGTCAGAGACAGTCTTCCTGCCCAGGGAAGACCACCTTTTCCGCAAGTTCCACTATCTCCCCTTCCTGCCCTC
AACTGAGGACGTTTACGACTGCAGGGTGGAGCACTGGGGCTTGGATGAGCCTCTTCTCAAGCACTGGGAGTTTG
ATGCTCCAAGCCCTCTCCCAGAGACTACAGAGAACGGCGGCGGAGGCAGCCTGGAAATCAGAGCCGCCTTCCTG
CGGCAGAGAAACACCGCCCTGAGAACCGAAGTGGCCGAGCTGGAACAGGAAGTGCAGCGGCTGGAAAACGAGGT
GTCCCAGTACGAGACAAGATACGGCCCTCTGGGAGGCGGCAAGGGCTCTCACCACCACCATCACCATCATCATC
ACCATTGA
Signal Peptide (Fibroin light chain-derived)
MMRPIVLVLLFATSALA (SEQ ID NO: 9)

When the MEW class II molecule is complexed with an antigen peptide, the 10-30 amino acid long antigen peptide binds the peptide-binding groove and is presented extracellularly to CD4+ cells. Both the alpha- and beta-chains fold into two separate domains; alpha-1 and alpha-2 for the alpha polypeptide, and beta-1 and beta-2 for the beta polypeptide. The invariant residues at L114, V116, V143, L158, and M160 that are recognized and bound by CD4 are located in the beta-2 domain of the beta polypeptide. The open-ended peptide-binding groove which holds the presented antigen is found between the alpha-1 and beta-1 domains. Upon interaction with a CD4+ T cell, the MHC class II complex interacts with a T cell receptor (TCR) expressed on the surface of the T cell. In addition, the beta chain of the MHC class II molecule weakly interacts (K_{D}>2 mM) with CD4 expressed on the surface of the T cell. The canonical CD4 amino acid sequence (UniProt—P01730) is provided in Table 2 (SEQ ID NO: 10).

TABLE 2
Human CD4 Amino Acid Sequence
MNRGVPFRHLLLVLQLALLPAATQGKKVVLGKKGDTVELTCTASQKK
SIQFHWKNSNQIKILGNQGSFLTKGPSKLNDRADSRRSLWDQGNFPL
IIKNLKIEDSDTYICEVEDQKEEVQLLVFGLTANSDTHLLQGQSLTL
TLESPPGSSPSVQCRSPRGKNIQGGKTLSVSQLELQDSGTWTCTVLQ
NQKKVEFKIDIVVLAFQKASSIVYKKEGEQVEFSFPLAFTVEKLTGS
GELWWQAERASSSKSWITFDLKNKEVSVKRVTQDPKLQMGKKLPLHL
TLPQALPQYAGSGNLTLALEAKTGKLHQEVNLVVMRATQLQKNLTCE
VWGPTSPKLMLSLKLENKEAKVSKREKAVWVLNPEAGMWQCLLSDSG
QVLLESNIKVLPTWSTPVQPMALIVLGGVAGLLLFIGLGIFFCVRCR
HRRRQAERMSQIKRLLSEKKTCQCPHRFQKTCSPI
(SEQ ID NO: 10)

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs, full-length TCRs and antigen binding portions thereof, chimeric TCRs, TCR fusion constructs, and synthetic TCRs. In human, TCRs are expressed on the surface of T cells, and they are responsible for T cell recognition and targeting of antigen presenting cells. Antigen presenting cells (APCs) display fragments of foreign proteins (antigens) complexed with the major histocompatibility complex (MHC class I or MHC class II; also referred to herein as complexed with an HLA molecule, e.g., an HLA class II molecule). A TCR recognizes and binds to the peptide:HLA complex and recruits CD8 (for MHC Class I molecules) or CD4 (for MHC class II molecules) expressed by T cells, activating the TCR. The activated TCR initiates downstream signaling and an immune response, including the destruction of the APC.

In general, a TCR can comprise two chains, an alpha chain and a beta chain (or less commonly a gamma chain and a delta chain), interconnected by disulfide bonds. Each chain comprises a variable domain (alpha chain variable domain and beta chain variable domain) and a constant region (alpha chain constant region and beta chain constant region). The variable domain is located distal to the cell membrane, and the variable domain interacts with an antigen. The constant region is located proximal to the cell membrane. A TCR can further comprises a transmembrane region and a short cytoplasmic tail. As used herein, the term “constant region” encompasses the transmembrane region and the cytoplasmic tail, when present, as well as the traditional “constant region.”

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region, while CDR1 and CDR2 are believed to primarily recognize the HLA molecule.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD4, as described herein), or a TCR described herein that is free from a cell membrane (e.g., an isolated TCR or a soluble TCR).

An “antigen binding molecule,” “portion of a TCR,” or “TCR fragment” refers to any portion of an TCR less than the whole. An antigen binding molecule can include the antigenic CDRs.

An “antigen” refers to any molecule, e.g., a peptide, that provokes an immune response or is capable of being bound by a TCR. An “epitope,” as used herein, refers to a portion of a polypeptide that provokes an immune response or is capable of being bound by a TCR. The immune response may involve either antibody production, or the activation of specific immunologically-competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or an epitope can be specific to a certain tissue, such as a diseased cell, e.g., a cancer cell, or it can be broadly expressed. In addition, fragments of larger molecules can act as antigens. In one aspect, antigens are tumor antigens. An epitope can be present in a longer polypeptide (e.g., in a protein), or an epitope can be present as a fragment of a longer polypeptide. In some aspects, an epitope is complexed with a major histocompatibility complex (MHC; also referred to herein as complexed with an HLA molecule, e.g., an HLA class 1 molecule).

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some aspects, the methods of the present invention can be used to reduce the tumor size of a tumor derived from, for example, melanoma, bone cancer, pancreatic cancer, skin cancer, cancer of the head or neck, uterine cancer, ovarian cancer, rectal cancer, stomach cancer, uterine cancer, lung cancer, Hodgkin's Disease, non-Hodgkin's lymphoma (NHL), cancer of the esophagus, cancer of the small intestine, cancer of the urethra, chronic or acute leukemia, acute myeloid leukemia, chronic myeloid leukemia, acute lymphoblastic leukemia (ALL) (including non T cell ALL), chronic lymphocytic leukemia (CLL), cancer of the bladder, cancer of the kidney or ureter, carcinoma of the renal pelvis, glioma, squamous cell cancer, and combinations of said cancers. The particular cancer can be responsive to chemo- or radiation therapy or the cancer can be refractory. A refractory cancer refers to a cancer that is not amendable to surgical intervention, and the cancer is either initially unresponsive to chemo- or radiation therapy or the cancer becomes unresponsive over time.

The term “progression-free survival,” which can be abbreviated as PFS, as used herein refers to the time from the treatment date to the date of disease progression per the revised IWG Response Criteria for Malignant Lymphoma or death from any cause.

The term “overall survival,” which can be abbreviated as OS, is defined as the time from the date of treatment to the date of death.

The term “infection,” as used herein refers to any type of invasion of one or more tissue of the body by a foreign agent. The term “infection” includes without limitation infection by a virus (including viroids and prions), a bacterium, a fungus, a parasite, and any combination thereof.

The term “lymphocyte” as used herein includes natural killer (NK) cells, T cells, or B cells. NK cells are a type of cytotoxic (cell toxic) lymphocyte that represents a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). T-cell receptors (TCR) differentiate T cells from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

The terms “modified” and “mutated,” when used herein to refer to a nucleotide or amino acid sequence, refers to a change in the sequence relative to a wild-type sequence or a specified reference sequence. The terms “modified” and “mutated” do not require a step in a process for making the modified or mutated sequence (e.g., the modified beta chain sequence), unless otherwise specified. Rather, these terms indicate that there is a variation in the modified or mutated sequence relative to a reference sequence, e.g., a wild-type sequence. For example, a DR beta chain comprising a substitution mutation at a position corresponding to amino acid residue 114 of SEQ ID NO: 1 does not require that a wild-type DR beta chain has been physically altered to arrive at the recited DR beta chain; but rather that, when properly aligned, the recited DR beta chain comprises an amino acid residue at the recited position (residue 114) that is different from the amino acid residue at the corresponding position in a wild-type or reference DR beta chain.

The term “any amino acid,” as used herein, means any known amino acid. Amino acids are organic compounds comprising (i) an amine (—NH₂) functional group, (ii) a carboxyl (—COOH) functional group, and (iii) a side chain (R group), wherein the side chain is specific to each amino acid. This includes but is not limited to any naturally occurring amino acid, as well as any modifications and variants thereof. There are about 500 naturally occurring amino acids, 20 of which are encoded by the genetic code. Amino acids with positively charged side chains include arginine (Arg; R), histidine (His, H), and lysine (Lys; K). Amino acids with a negatively charged side chain include aspartic acid (Asp; D) and glutamic acid (Glu; E). Amino acids with a polar uncharged side chain include serine (Ser; S), threonine (Thr; T), glutamine (Gln; Q), and asparagine (Asn; N). Amino acids with a hydrophobic side chain include alanine (Ala; A), isoleucine (Ile; I), leucine (Leu; L), methionine (Met; M), phenylalanine (Phe; F), valine (Val; V), Tryptophan (Trp; W), Tyrosine (Tyr; Y). Tryptophan (Trp; W), tyrosine (Tyr; Y), and methionine (Met; M) can also be classified as polar and/or amphipathic, in that these amino acids can often be found at the surface of proteins or lipid membranes. Additional amino acids include cysteine (Cys; C), selenocysteine (Sec; U), glycine (Gly; G) and proline (Pro; P).

As used herein “at a position corresponding to” is used as a means to identify a particular amino acid residue, e.g., a specific amino acid position, in a polynucleotide or a particular nucleic acid, e.g., a specific nucleic acid position, in a polypeptide. The position can be determined by properly aligning the sequence in question with the referenced sequence. A person of skill in the art would readily understand how to align to sequences to determine the relative position. For example, various alignment tools are available online, including, without limitation, “Clustal Omega Multiple Sequence Alignment,” available at www.ebi.ac.uk (last visited May 25, 2019).

The term “genetically engineered” or “engineered” refers to a method of modifying the genome of a cell, including, but not limited to, deleting a coding or non-coding region or a portion thereof or inserting a coding region or a portion thereof. In some aspects, the cell that is modified is a lymphocyte, e.g., a T cell or a modified cell that expresses CD4, which can either be obtained from a patient or a donor. The cell can be modified to express an exogenous construct, such as, e.g., a T cell receptor (TCR) disclosed herein, which is incorporated into the cell's genome. In some aspects, the cell is modified to express CD4.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation.

Cells used in an immunotherapy described herein can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety. An immunotherapy can also comprise administering a modified cell to a subject, wherein the modified cell expresses CD4 and a TCR disclosed herein. In some aspects, the modified cell is not a T cell.

A “patient” as used herein includes any human who is afflicted with a cancer (e.g., a lymphoma or a leukemia). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD4 complex, that specifically binds with a cognate stimulatory ligand present on an antigen present cell. A “stimulatory ligand” is a ligand that when present on an antigen presenting cell (e.g., an aAPC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an WIC Class II molecule loaded with a peptide, an anti-CD4 antibody, an anti-CD28 antibody, an anti-CD2 antibody, and an anti-CD3 antibody.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Various aspects of the invention are described in further detail in the following subsections.

### II. Compositions of the Disclosure

The present disclosure is directed to HLA class II molecules with enhanced CD4 binding. Certain aspects of the present disclosure are directed to HLA class II molecules comprising a beta chain, wherein the beta chain comprises one or more mutations. In certain aspects, the one or more mutations in the beta chain increase the affinity of the beta chain for CD4. In certain aspects, the beta chain is an HLA-DR (“DR”) beta chain.

II.A. MHC Class II Molecules

The human leukocyte antigen (HLA) system (the major histocompatibility complex [MHC] in humans) is an important part of the immune system and is controlled by genes located on chromosome 6. It encodes cell surface molecules specialized to present antigenic peptides to the T-cell receptor (TCR) on T cells. (See also Overview of the Immune System.) MHC molecules that present antigen (Ag) are divided into 2 main classes: Class I MHC molecules and Class II MHC molecules.

Class II MHC molecules are present as transmembrane glycoproteins on the surface of professional antigen presenting cells (APCs). Intact class II molecules consist of an alpha chain and a beta chain. Three loci in the HLA complex encode MHC class II proteins: HLA-DP, HLA-DQ, and HLA-DR. T cells that express CD4 molecules react with class II MHC molecules. These lymphocytes often have effector and helper functions and activate a response to eliminate self-cells infected with intracellular pathogens or to destroy extracellular parasites and help other T cells such as CD8 T cells. Because only professional APCs express class II MHC molecules, only these cells present antigen for CD4 T cells (CD4 binds to the nonpolymorphic part of the alpha-2 and beta-2 domains of the alpha and beta chains of an MHC class II molecule respectively).

In some aspects, the HLA class II alpha and beta chains are selected from an HLA-DP, HLA-DQ, and HLA-DR allele. In certain aspects, the HLA class II beta chain is an HLA-DR allele. In certain aspects, the HLA class II alpha chain is an HLA-DR allele.

Many HLA-DR alleles are known in the art, and any of the known alleles can be used in the present disclosure. Examples of HLA-DR alpha chain and beta chain alleles are shown in Table 1. An updated list of HLA alleles is available at hla.alleles.org/(last visited on Jul. 10, 2019).

II.A.1. MHC Class II Beta Chain

In certain aspects, the HLA class II molecule comprises a DR beta chain, wherein the DR beta chain comprises an amino acid other than leucine at a position corresponding to amino acid residue 114 of SEQ ID NO: 1. Any amino acid other than leucine can be present at the position corresponding to amino acid residue 114 of SEQ ID NO: 1. In some aspects, the amino acid other than leucine is an amino acid comprising a hydrophobic side chain. In certain aspects, the amino acid other than leucine at the position corresponding to amino acid residue 114 of SEQ ID NO: 1 is an amino acid selected from an alanine, a valine, an isoleucine, a methionine, a phenylalanine, a tyrosine, and a tryptophan. In certain aspects, the amino acid other than leucine at the position corresponding to amino acid residue 114 of SEQ ID NO: 1 is an alanine. In certain aspects, the amino acid other than leucine at the position corresponding to amino acid residue 114 of SEQ ID NO: 1 is a valine. In certain aspects, the amino acid other than leucine at the position corresponding to amino acid residue 114 of SEQ ID NO: 1 is an isoleucine. In certain aspects, the amino acid other than leucine at the position corresponding to amino acid residue 114 of SEQ ID NO: 1 is a methionine. In certain aspects, the amino acid other than leucine at the position corresponding to amino acid residue 114 of SEQ ID NO: 1 is a phenylalanine. In certain aspects, the amino acid other than leucine at the position corresponding to amino acid residue 114 of SEQ ID NO: 1 is a tyrosine. In certain aspects, the amino acid other than leucine at the position corresponding to amino acid residue 114 of SEQ ID NO: 1 is a tryptophan.

In some embodiments, the amino acid other than leucine at the position corresponding to amino acid residue 114 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than leucine at the position corresponding to amino acid residue 114 of SEQ ID NO: 1 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than valine at a position corresponding to amino acid residue 143 of SEQ ID NO: 1. Any amino acid other than valine can be present at the position corresponding to amino acid residue 143 of SEQ ID NO: 1. In some aspects, the amino acid other than valine is an amino acid comprising a hydrophobic side chain. In certain aspects, the amino acid other than valine at the position corresponding to amino acid residue 143 of SEQ ID NO: 1 is an amino acid selected from an alanine, an isoleucine, a leucine, a methionine, a phenylalanine, a tyrosine, and a tryptophan. In certain aspects, the amino acid other than valine at the position corresponding to amino acid residue 143 of SEQ ID NO: 1 is an alanine. In certain aspects, the amino acid other than valine at the position corresponding to amino acid residue 143 of SEQ ID NO: 1 is an isoleucine. In certain aspects, the amino acid other than valine at the position corresponding to amino acid residue 143 of SEQ ID NO: 1 is a leucine. In certain aspects, the amino acid other than valine at the position corresponding to amino acid residue 143 of SEQ ID NO: 1 is a methionine. In certain aspects, the amino acid other than valine at the position corresponding to amino acid residue 143 of SEQ ID NO: 1 is a phenylalanine. In certain aspects, the amino acid other than valine at the position corresponding to amino acid residue 143 of SEQ ID NO: 1 is a tyrosine. In certain aspects, the amino acid other than valine at the position corresponding to amino acid residue 143 of SEQ ID NO: 1 is a tryptophan.

In some aspects, the amino acid other than valine at the position corresponding to amino acid residue 143 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than valine at the position corresponding to amino acid residue 143 of SEQ ID NO: 1 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than serine at a position corresponding to amino acid residue 118 of SEQ ID NO: 1. Any amino acid other than serine can be present at the position corresponding to amino acid residue 118 of SEQ ID NO: 1. In some aspects, the amino acid other than serine is an amino acid comprising an electrically charged side chain. In certain aspects, the amino acid other than serine at the position corresponding to amino acid residue 118 of SEQ ID NO: 1 is an amino acid selected from an arginine, a histidine, and a lysine. In certain aspects, the amino acid other than serine at the position corresponding to amino acid residue 118 of SEQ ID NO: 1 is an arginine. In certain aspects, the amino acid other than serine at the position corresponding to amino acid residue 118 of SEQ ID NO: 1 is a histidine. In certain aspects, the amino acid other than serine at the position corresponding to amino acid residue 118 of SEQ ID NO: 1 is a lysine.

In some aspects, the amino acid other than serine at the position corresponding to amino acid residue 118 of SEQ ID NO: 1 consists of more than one amino acid, e.g., two amino acids, three amino acids, four amino acids, five amino acids, or more. In some aspects at least one of the more than one amino acids comprises an electrically charged side chain. In certain aspects, the amino acid other than serine at the position corresponding to amino acid residue 118 of SEQ ID NO: 1 consists of a series, e.g., at least 2, at least 3, at least 4, or at least 5, amino acids, wherein each of the series of amino acids comprises an electrically charged side chain.

In certain aspects, the HLA class II molecule comprises a DR beta chain, wherein the DR beta chain comprises an amino acid other than threonine at a position corresponding to amino acid residue 157 of SEQ ID NO: 1. Any amino acid other than threonine can be present at the position corresponding to amino acid residue 157 of SEQ ID NO: 1. In some aspects, the amino acid other than threonine is an amino acid comprising a hydrophobic side chain. In certain aspects, the amino acid other than threonine at the position corresponding to amino acid residue 157 of SEQ ID NO: 1 is an amino acid selected an alanine, a valine, an isoleucine, a leucine, a methionine, a phenylalanine, a tyrosine, and a tryptophan. In certain aspects, the amino acid other than threonine at the position corresponding to amino acid residue 157 of SEQ ID NO: 1 is an alanine. In certain aspects, the amino acid other than threonine at the position corresponding to amino acid residue 157 of SEQ ID NO: 1 is a valine. In certain aspects, the amino acid other than threonine at the position corresponding to amino acid residue 157 of SEQ ID NO: 1 is an isoleucine. In certain aspects, the amino acid other than threonine at the position corresponding to amino acid residue 157 of SEQ ID NO: 1 is a leucine. In certain aspects, the amino acid other than threonine at the position corresponding to amino acid residue 157 of SEQ ID NO: 1 is a methionine. In certain aspects, the amino acid other than threonine at the position corresponding to amino acid residue 157 of SEQ ID NO: 1 is a phenylalanine. In certain aspects, the amino acid other than threonine at the position corresponding to amino acid residue 157 of SEQ ID NO: 1 is a tyrosine. In certain aspects, the amino acid other than threonine at the position corresponding to amino acid residue 157 of SEQ ID NO: 1 is a tryptophan.

In some aspects, the amino acid other than threonine at the position corresponding to amino acid residue 157 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than threonine at the position corresponding to amino acid residue 157 of SEQ ID NO: 1 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than lysine at a position corresponding to amino acid residue 139 of SEQ ID NO: 1. Any amino acid other than lysine can be present at the position corresponding to amino acid residue 139 of SEQ ID NO: 1. In some aspects, the amino acid other than lysine is an amino acid comprising a polar uncharged side chain. In certain aspects, the amino acid other than lysine at the position corresponding to amino acid residue 139 of SEQ ID NO: 1 is an amino acid selected from a serine, a threonine, and a glutamine. In certain aspects, the amino acid other than lysine at the position corresponding to amino acid residue 139 of SEQ ID NO: 1 is a serine. In certain aspects, the amino acid other than lysine at the position corresponding to amino acid residue 139 of SEQ ID NO: 1 is a threonine. In certain aspects, the amino acid other than lysine at the position corresponding to amino acid residue 139 of SEQ ID NO: 1 is a glutamine.

In some aspects, the amino acid other than lysine at the position corresponding to amino acid residue 139 of SEQ ID NO: 1 consists of more than one amino acid, e.g., two amino acids, three amino acids, four amino acids, five amino acids, or more. In some aspects at least one of the more than one amino acids comprises a polar uncharged side chain. In certain aspects, the amino acid other than lysine at the position corresponding to amino acid residue 139 of SEQ ID NO: 1 consists of a series, e.g., at least 2, at least 3, at least 4, or at least 5, amino acids, wherein each of the series of amino acids comprises a polar uncharged side chain.

In certain aspects, the HLA class II molecule comprises a DR beta chain, wherein the DR beta chain comprises an amino acid other than glycine at a position corresponding to amino acid residue 146 of SEQ ID NO: 1. Any amino acid other than glycine can be present at the position corresponding to amino acid residue 146 of SEQ ID NO: 1. In some aspects, the amino acid other than glycine is an amino acid comprising a polar uncharged side chain. In certain aspects, the amino acid other than glycine at the position corresponding to amino acid residue 146 of SEQ ID NO: 1 is an amino acid selected from a serine, an asparagine, a threonine, and a glutamine. In certain aspects, the amino acid other than glycine at the position corresponding to amino acid residue 146 of SEQ ID NO: 1 is a serine. In certain aspects, the amino acid other than glycine at the position corresponding to amino acid residue 146 of SEQ ID NO: 1 is an asparagine. In certain aspects, the amino acid other than glycine at the position corresponding to amino acid residue 146 of SEQ ID NO: 1 is a threonine. In certain aspects, the amino acid other than glycine at the position corresponding to amino acid residue 146 of SEQ ID NO: 1 is a glutamine.

In some aspects, the amino acid other than glycine at the position corresponding to amino acid residue 146 of SEQ ID NO: 1 consists of more than one amino acid, e.g., two amino acids, three amino acids, four amino acids, five amino acids, or more. In some aspects at least one of the more than one amino acids comprises a polar uncharged side chain. In certain aspects, the amino acid other than glycine at the position corresponding to amino acid residue 146 of SEQ ID NO: 1 consists of a series, e.g., at least 2, at least 3, at least 4, or at least 5, amino acids, wherein each of the series of amino acids comprises a polar uncharged side chain.

In certain aspects, the HLA class II molecule comprises a DR beta chain, wherein the DR beta chain comprises an amino acid other than threonine at a position corresponding to amino acid residue 163 of SEQ ID NO: 1. Any amino acid other than threonine can be present at the position corresponding to amino acid residue 163 of SEQ ID NO: 1. In some aspects, the amino acid other than threonine is an amino acid comprising a hydrophobic side chain. In certain aspects, the amino acid other than threonine at the position corresponding to amino acid residue 163 of SEQ ID NO: 1 is an amino acid selected from an alanine, a valine, an isoleucine, a leucine, a methionine, a phenylalanine, a tyrosine, and a tryptophan. In certain aspects, the amino acid other than threonine at the position corresponding to amino acid residue 163 of SEQ ID NO: 1 is an alanine. In certain aspects, the amino acid other than threonine at the position corresponding to amino acid residue 163 of SEQ ID NO: 1 is a valine. In certain aspects, the amino acid other than threonine at the position corresponding to amino acid residue 163 of SEQ ID NO: 1 is an isoleucine. In certain aspects, the amino acid other than threonine at the position corresponding to amino acid residue 163 of SEQ ID NO: 1 is a leucine. In certain aspects, the amino acid other than threonine at the position corresponding to amino acid residue 163 of SEQ ID NO: 1 is a methionine. In certain aspects, the amino acid other than threonine at the position corresponding to amino acid residue 163 of SEQ ID NO: 1 is a phenylalanine. In certain aspects, the amino acid other than threonine at the position corresponding to amino acid residue 163 of SEQ ID NO: 1 is a tyrosine. In certain aspects, the amino acid other than threonine at the position corresponding to amino acid residue 163 of SEQ ID NO: 1 is a tryptophan.

In some aspects, the amino acid other than threonine at the position corresponding to amino acid residue 163 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than threonine at the position corresponding to amino acid residue 163 of SEQ ID NO: 1 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than valine at a position corresponding to amino acid residue 164 of SEQ ID NO: 1. Any amino acid other than valine can be present at the position corresponding to amino acid residue 164 of SEQ ID NO: 1. In some aspects, the amino acid other than valine is an amino acid comprising a polar uncharged side chain. In certain aspects, the amino acid other than valine at the position corresponding to amino acid residue 164 of SEQ ID NO: 1 is an amino acid selected from a serine, an asparagine, a threonine, and a glutamine. In certain aspects, the amino acid other than valine at the position corresponding to amino acid residue 164 of SEQ ID NO: 1 is a serine. In certain aspects, the amino acid other than valine at the position corresponding to amino acid residue 164 of SEQ ID NO: 1 is an asparagine. In certain aspects, the amino acid other than valine at the position corresponding to amino acid residue 164 of SEQ ID NO: 1 is a threonine. In certain aspects, the amino acid other than valine at the position corresponding to amino acid residue 164 of SEQ ID NO: 1 is a glutamine.

In some aspects, the amino acid other than valine at the position corresponding to amino acid residue 164 of SEQ ID NO: 1 consists of more than one amino acid, e.g., two amino acids, three amino acids, four amino acids, five amino acids, or more. In some aspects at least one of the more than one amino acids comprises a polar uncharged side chain. In certain aspects, the amino acid other than valine at the position corresponding to amino acid residue 164 of SEQ ID NO: 1 consists of a series, e.g., at least 2, at least 3, at least 4, or at least 5, amino acids, wherein each of the series of amino acids comprises a polar uncharged side chain.

In certain aspects of the present disclosure, the MEW class II molecule comprises a DR beta chain comprising more than one substitution mutation relative to the wild-type DR beta chain. In certain aspects, the DR beta chain comprises at least two mutations, at least three mutations, at least four mutations, at least five mutations, at least six mutations, at least seven mutations, at least eight mutations, at least nine mutations, or at least ten mutations relative to the wild-type DR beta chain.

In certain aspects, the DR beta chain comprises an amino acid other than leucine at a position corresponding to amino acid residue 114 of SEQ ID NO: 1 and an amino acid other than valine at a position corresponding to amino acid residue 143 of SEQ ID NO: 1.

In certain aspects, the DR beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, and (d) an amino acid other than threonine at a position corresponding to amino acid residue 157 of SEQ ID NO: 1.

In certain aspects, the DR beta chain comprises an amino acid other than leucine at a position corresponding to amino acid residue 114 of SEQ ID NO: 1; an amino acid other than valine at a position corresponding to amino acid residue 143 of SEQ ID NO: 1; and at least two of: (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain aspects, the DR beta chain comprises an amino acid other than leucine at a position corresponding to amino acid residue 114 of SEQ ID NO: 1; an amino acid other than valine at a position corresponding to amino acid residue 143 of SEQ ID NO: 1; and at least three of: (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain aspects, the DR beta chain comprises an amino acid other than leucine at a position corresponding to amino acid residue 114 of SEQ ID NO: 1; an amino acid other than valine at a position corresponding to amino acid residue 143 of SEQ ID NO: 1; and at least four of: (i) an amino acid other than serine at a position corresponding to amino acid residue 118 of SEQ ID NO: 1, (ii) an amino acid other than lysine at a position corresponding to amino acid residue 139 of SEQ ID NO: 1, (iii) an amino acid other than glycine at a position corresponding to amino acid residue 146 of SEQ ID NO: 1, (iv) an amino acid other than threonine at a position corresponding to amino acid residue 157 of SEQ ID NO: 1, (v) an amino acid other than threonine at a position corresponding to amino acid residue 163 of SEQ ID NO: 1, and (vi) an amino acid other than valine at a position corresponding to amino acid residue 164 of SEQ ID NO: 1.

In certain aspects, the DR beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, and (d) an amino acid other than threonine at a position corresponding to amino acid residue 157 of SEQ ID NO: 1; and at least one of: (i) an amino acid other than lysine at a position corresponding to amino acid residue 139 of SEQ ID NO: 1, (ii) an amino acid other than glycine at a position corresponding to amino acid residue 146 of SEQ ID NO: 1, (iii) an amino acid other than threonine at a position corresponding to amino acid residue 163 of SEQ ID NO: 1, and (iv) an amino acid other than valine at a position corresponding to amino acid residue 164 of SEQ ID NO: 1.

In certain aspects, the DR beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, and (d) an amino acid other than threonine at a position corresponding to amino acid residue 157 of SEQ ID NO: 1; and at least two of: (i) an amino acid other than lysine at a position corresponding to amino acid residue 139 of SEQ ID NO: 1, (ii) an amino acid other than glycine at a position corresponding to amino acid residue 146 of SEQ ID NO: 1, (iii) an amino acid other than threonine at a position corresponding to amino acid residue 163 of SEQ ID NO: 1, and (iv) an amino acid other than valine at a position corresponding to amino acid residue 164 of SEQ ID NO: 1.

In certain aspects, the DR beta chain comprises (a) an amino acid other than leucine at a position corresponding to amino acid residue 114 of SEQ ID NO: 1, (b) an amino acid other than valine at a position corresponding to amino acid residue 143 of SEQ ID NO: 1, (c) an amino acid other than serine at a position corresponding to amino acid residue 118 of SEQ ID NO: 1, and (d) an amino acid other than threonine at a position corresponding to amino acid residue 157 of SEQ ID NO: 1; and at least three of: (i) an amino acid other than lysine at a position corresponding to amino acid residue 139 of SEQ ID NO: 1, (ii) an amino acid other than glycine at a position corresponding to amino acid residue 146 of SEQ ID NO: 1, (iii) an amino acid other than threonine at a position corresponding to amino acid residue 163 of SEQ ID NO: 1, and (iv) an amino acid other than valine at a position corresponding to amino acid residue 164 of SEQ ID NO: 1.

In certain aspects, the DR beta chain comprises (a) an amino acid other than leucine at a position corresponding to amino acid residue 114 of SEQ ID NO: 1; (b) an amino acid other than valine at a position corresponding to amino acid residue 143 of SEQ ID NO: 1; (c) an amino acid other than serine at a position corresponding to amino acid residue 118 of SEQ ID NO: 1, (d) an amino acid other than lysine at a position corresponding to amino acid residue 139 of SEQ ID NO: 1, (e) an amino acid other than glycine at a position corresponding to amino acid residue 146 of SEQ ID NO: 1, (f) an amino acid other than threonine at a position corresponding to amino acid residue 157 of SEQ ID NO: 1, (g) an amino acid other than threonine at a position corresponding to amino acid residue 163 of SEQ ID NO: 1, and (h) an amino acid other than valine at a position corresponding to amino acid residue 164 of SEQ ID NO: 1.

In certain aspects, the DR beta chain comprises (a) a tryptophan at a position corresponding to amino acid residue 114 of SEQ ID NO: 1, (b) a methionine at a position corresponding to amino acid residue 143 of SEQ ID NO: 1, (c) a histidine at a position corresponding to amino acid residue 118 of SEQ ID NO: 1, and (d) an isoleucine at a position corresponding to amino acid residue 157 of SEQ ID NO: 1.

In some aspects, (i) the amino acid other than leucine at the position corresponding to amino acid residue 114 of SEQ ID NO: 1, (ii) the amino acid other than valine at a position corresponding to amino acid residue 143 of SEQ ID NO: 1, or each of the amino acid other than leucine at the position corresponding to amino acid residue 114 of SEQ ID NO: 1 and the amino acid other than valine at a position corresponding to amino acid residue 143 of SEQ ID NO: 1 is an amino acid comprising a hydrophobic side chain.

In some aspects, (i) the amino acid other than leucine at the position corresponding to amino acid residue 114 of SEQ ID NO: 1 is selected from an alanine, a valine, an isoleucine, a methionine, a phenylalanine, a tyrosine, and a tryptophan; (ii) the amino acid other than valine at a position corresponding to amino acid residue 143 of SEQ ID NO: 1 is selected from an alanine, an isoleucine, a leucine, a methionine, a phenylalanine, a tyrosine, and a tryptophan; (iii) the amino acid other than serine at a position corresponding to amino acid residue 118 of SEQ ID NO: 1 is selected from an arginine, a histidine, and a lysine; and/or (iv) the amino acid other than threonine at a position corresponding to amino acid residue 157 of SEQ ID NO: 1 is selected from an alanine, a valine, an isoleucine, a leucine, a methionine, a phenylalanine, a tyrosine, and a tryptophan.

In some aspects, (i) the amino acid other than leucine at the position corresponding to amino acid residue 114 of SEQ ID NO: 1 is selected from an alanine, a valine, an isoleucine, a methionine, a phenylalanine, a tyrosine, and a tryptophan; (ii) the amino acid other than valine at a position corresponding to amino acid residue 143 of SEQ ID NO: 1 is selected from an alanine, an isoleucine, a leucine, a methionine, a phenylalanine, a tyrosine, and a tryptophan; (iii) the amino acid other than serine at a position corresponding to amino acid residue 118 of SEQ ID NO: 1 is selected from an arginine, a histidine, and a lysine; (iv) the amino acid other than threonine at a position corresponding to amino acid residue 157 of SEQ ID NO: 1 is selected from an alanine, a valine, an isoleucine, a leucine, a methionine, a phenylalanine, a tyrosine, and a tryptophan; (v) the amino acid other than lysine at a position corresponding to amino acid residue 139 of SEQ ID NO: 1 is selected from a serine, a threonine, and a glutamine; (vi) the amino acid other than glycine at a position corresponding to amino acid residue 146 of SEQ ID NO: 1 is selected from a serine, an asparagine, a threonine, and a glutamine; (vii) the amino acid other than threonine at a position corresponding to amino acid residue 163 of SEQ ID NO: 1 is selected from an alanine, a valine, an isoleucine, a leucine, a methionine, a phenylalanine, a tyrosine, and a tryptophan; and/or (viii) the amino acid other than valine at a position corresponding to amino acid residue 164 of SEQ ID NO: 1 is selected from a serine, an asparagine, a threonine, and a glutamine.

In certain aspects, a DR beta chain described herein has an increased affinity for a CD4 protein as compared to a reference HLA class II molecule. In some aspects, the reference HLA class II molecule is an HLA class II molecule having a wild-type DR beta chain. In some aspects, the reference HLA class II molecule is an HLA class II molecule having a DR beta chain comprising (i) a leucine at a position corresponding to amino acid residue 114 of SEQ ID NO: 1 and/or (ii) a valine at a position corresponding to amino acid residue 143 of SEQ ID NO: 1. In some aspects, the reference HLA class II molecule is an HLA class II molecule having a DR beta chain comprising (i) a leucine at a position corresponding to amino acid residue 114 of SEQ ID NO: 1, (ii) a valine at a position corresponding to amino acid residue 143 of SEQ ID NO: 1, (iii) a serine at a position corresponding to amino acid residue 118 of SEQ ID NO: 1, and (iv) a threonine at a position corresponding to amino acid residue 157 of SEQ ID NO: 1.

In some aspects, the increased affinity for CD4 is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, at least about 4000-fold, at least about 4500-fold, or at least about 4000-fold greater than the affinity of the reference HLA class II molecule for CD4.

In some aspects, the increased affinity for CD4 is at least about 1.5-fold to at least about 5000-fold, 1.5-fold to at least about 4000-fold, 1.5-fold to at least about 3000-fold, 1.5-fold to at least about 2000-fold, 1.5-fold to at least about 1000-fold, 10-fold to at least about 5000-fold, 10-fold to at least about 4000-fold, 10-fold to at least about 3000-fold, 10-fold to at least about 2000-fold, 10-fold to at least about 1000-fold, 10-fold to at least about 900-fold, 10-fold to at least about 800-fold, 10-fold to at least about 700-fold, 10-fold to at least about 600-fold, 10-fold to at least about 500-fold, 10-fold to at least about 400-fold, 10-fold to at least about 300-fold, 10-fold to at least about 200-fold, 10-fold to at least about 100-fold, 100-fold to at least about 5000-fold, 100-fold to at least about 4000-fold, 100-fold to at least about 3000-fold, 100-fold to at least about 2000-fold, 100-fold to at least about 1000-fold, 100-fold to at least about 900-fold, 100-fold to at least about 800-fold, 100-fold to at least about 700-fold, 100-fold to at least about 600-fold, 100-fold to at least about 500-fold, 100-fold to at least about 400-fold, 100-fold to at least about 300-fold, or 100-fold to at least about 200-fold greater than the affinity of the reference HLA class II molecule for CD4.

In certain aspects, the DR beta chain comprises an allele selected from an HLA-DRB1*01, an HLA-DRB1*03, an HLA-DRB1*04, an HLA-DRB1*06, an HLA-DRB1*07, an HLA-DRB1*08, an HLA-DRB1*09, an HLA-DRB1*10, an HLA-DRB1*11, an HLA-DRB1*12, an HLA-DRB1*13, an HLA-DRB1*14, an HLA-DRB1*15, or an HLA-DRB1*16 allele. In some aspects, the DR beta chain comprises an HLA-DRB1*01 allele. In particular aspects, the DR beta chain comprises an HLA-DRB1*01:01 allele.

In certain aspects, the DR beta chain comprises an allele selected from DRB1*01:01:01, DRB1*01:01:02, DRB1*01:01:03, DRB1*01:01:04, DRB1*01:01:05, DRB1*01:01:06, DRB1*01:01:07, DRB1*01:01:08, DRB1*01:01:09, DRB1*01:01:10, DRB1*01:01:11, DRB1*01:01:12, DRB1*01:01:13, DRB1*01:01:14, DRB1*01:01:15, DRB1*01:01:16, DRB1*01:01:17, DRB1*01:01:18, DRB1*01:01:19, DRB1*01:01:20, DRB1*01:01:21, DRB1*01:01:22, DRB1*01:01:23, DRB1*01:01:24, DRB1*01:01:25, DRB1*01:01:26, DRB1*01:01:27, DRB1*01:01:28, DRB1*01:01:29, DRB1*01:01:30, DRB1*01:01:31, DRB1*01:01:32, DRB1*01:01:33, DRB1*01:02:01:01, DRB1*01:02:01:02, DRB1*01:02:02, DRB1*01:02:03, DRB1*01:02:04, DRB1*01:02:05, DRB1*01:02:06, DRB1*01:02:07, DRB1*01:02:08, DRB1*01:02:09, DRB1*01:02:10, DRB1*01:02:11, DRB1*01:02:12, DRB1*01:02:13, DRB1*01:03:01, DRB1*01:03:02, DRB1*01:03:03, DRB1*01:03:04, DRB1*01:04, DRB1*01:05, DRB1*01:06, DRB1*01:07, DRB1*01:08, DRB1*01:09, DRB1*01:10, DRB1*01:100, DRB1*01:11:01, DRB1*01:11:02, DRB1*01:12, DRB1*01:13, DRB1*01:14, DRB1*01:15, DRB1*01:16, DRB1*01:17, DRB1*01:18:01, DRB1*01:18:02, DRB1*01:19, DRB1*01:20:01, DRB1*01:20:02, DRB1*01:21, DRB1*01:22, DRB1*01:23, DRB1*01:24:01, DRB1*01:24:02, DRB1*01:25, DRB1*01:26, DRB1*01:27, DRB1*01:28, DRB1*01:29:01, DRB1*01:29:02, DRB1*01:30, DRB1*01:31, DRB1*01:32, DRB1*01:33N, DRB1*01:34, DRB1*01:35, DRB1*01:36, DRB1*01:37, DRB1*01:38, DRB1*01:39N, DRB1*01:40N, DRB1*01:41, DRB1*01:42, DRB1*01:43, DRB1*01:44:01, DRB1*01:44:02, DRB1*01:45, DRB1*01:46, DRB1*01:47, DRB1*01:48, DRB1*01:49, DRB1*01:50, DRB1*01:51, DRB1*01:52N, DRB1*01:53, DRB1*01:54, DRB1*01:55, DRB1*01:56, DRB1*01:57, DRB1*01:58, DRB1*01:59, DRB1*01:60, DRB1*01:61, DRB1*01:62N, DRB1*01:63, DRB1*01:64, DRB1*01:65:01, DRB1*01:65:02, DRB1*01:66, DRB1*01:67, DRB1*01:68N, DRB1*01:69, DRB1*01:70, DRB1*01:71, DRB1*01:72, DRB1*01:73, DRB1*01:74, DRB1*01:75, DRB1*01:76, DRB1*01:77, DRB1*01:78, DRB1*01:79, DRB1*01:80, DRB1*01:81, DRB1*01:82, DRB1*01:83, DRB1*01:84, DRB1*01:85, DRB1*01:86, DRB1*01:87, DRB1*01:88, DRB1*01:89, DRB1*01:90, DRB1*01:91Q, DRB1*01:92, DRB1*01:93, DRB1*01:94, DRB1*01:95, DRB1*01:96, DRB1*01:97, DRB1*01:98, DRB1*01:99, DRB1*03:01:01:01, DRB1*03:01:01:02, DRB1*03:01:01:03, DRB1*03:01:02, DRB1*03:01:03, DRB1*03:01:04, DRB1*03:01:05, DRB1*03:01:06, DRB1*03:01:07, DRB1*03:01:08, DRB1*03:01:09, DRB1*03:01:10, DRB1*03:01:11, DRB1*03:01:12, DRB1*03:01:13, DRB1*03:01:14, DRB1*03:01:15, DRB1*03:01:16, DRB1*03:01:17, DRB1*03:01:18, DRB1*03:01:19, DRB1*03:01:20, DRB1*03:01:21, DRB1*03:01:22, DRB1*03:01:23, DRB1*03:01:24, DRB1*03:01:25, DRB1*03:01:26, DRB1*03:01:27, DRB1*03:01:28, DRB1*03:02:01, DRB1*03:02:02, DRB1*03:02:03, DRB1*03:03, DRB1*03:04:01, DRB1*03:04:02, DRB1*03:05:01, DRB1*03:05:02, DRB1*03:05:03, DRB1*03:06, DRB1*03:07:01, DRB1*03:07:02, DRB1*03:08, DRB1*03:09, DRB1*03:10, DRB1*03:100:01, DRB1*03:100:02, DRB1*03:101, DRB1*03:102, DRB1*03:103, DRB1*03:104, DRB1*03:105, DRB1*03:106, DRB1*03:107, DRB1*03:108, DRB1*03:109, DRB1*03:110, DRB1*03:111, DRB1*03:112, DRB1*03:113, DRB1*03:114, DRB1*03:115, DRB1*03:116, DRB1*03:117, DRB1*03:118, DRB1*03:119, DRB1*03:11:01, DRB1*03:12, DRB1*03:120, DRB1*03:121, DRB1*03:122, DRB1*03:123, DRB1*03:124, DRB1*03:125, DRB1*03:126, DRB1*03:127, DRB1*03:128, DRB1*03:129, DRB1*03:130, DRB1*03:131, DRB1*03:132, DRB1*03:133, DRB1*03:134, DRB1*03:135, DRB1*03:136, DRB1*03:137, DRB1*03:138, DRB1*03:139, DRB1*03:13:01, DRB1*03:13:02, DRB1*03:14, DRB1*03:140, DRB1*03:141, DRB1*03:142, DRB1*03:143, DRB1*03:144, DRB1*03:145, DRB1*03:146, DRB1*03:147, DRB1*03:148, DRB1*03:149, DRB1*03:150, DRB1*03:151, DRB1*03:152, DRB1*03:153, DRB1*03:154, DRB1*03:155, DRB1*03:156N, DRB1*03:157, DRB1*03:158, DRB1*03:15:01, DRB1*03:15:02, DRB1*03:16, DRB1*03:17, DRB1*03:18, DRB1*03:19, DRB1*03:20, DRB1*03:21, DRB1*03:22, DRB1*03:23, DRB1*03:24, DRB1*03:25:01, DRB1*03:25:02, DRB1*03:26, DRB1*03:27, DRB1*03:28, DRB1*03:29, DRB1*03:30, DRB1*03:31, DRB1*03:32, DRB1*03:33, DRB1*03:34, DRB1*03:35, DRB1*03:36, DRB1*03:37, DRB1*03:38, DRB1*03:39, DRB1*03:40, DRB1*03:41:01, DRB1*03:41:02, DRB1*03:42, DRB1*03:43, DRB1*03:44, DRB1*03:45, DRB1*03:46, DRB1*03:47, DRB1*03:48, DRB1*03:49, DRB1*03:50, DRB1*03:51, DRB1*03:52, DRB1*03:53, DRB1*03:54, DRB1*03:55, DRB1*03:56, DRB1*03:57, DRB1*03:58, DRB1*03:59, DRB1*03:60, DRB1*03:61, DRB1*03:62, DRB1*03:63, DRB1*03:64, DRB1*03:65, DRB1*03:66, DRB1*03:67N, DRB1*03:68N, DRB1*03:69, DRB1*03:70, DRB1*03:71:01, DRB1*03:71:02, DRB1*03:72, DRB1*03:73, DRB1*03:74, DRB1*03:75, DRB1*03:76, DRB1*03:77, DRB1*03:78, DRB1*03:79, DRB1*03:80, DRB1*03:81, DRB1*03:82, DRB1*03:83, DRB1*03:84, DRB1*03:85, DRB1*03:86, DRB1*03:87, DRB1*03:88, DRB1*03:89, DRB1*03:90, DRB1*03:91, DRB1*03:92, DRB1*03:93, DRB1*03:94, DRB1*03:95, DRB1*03:96, DRB1*03:97, DRB1*03:98, DRB1*03:99, DRB1*04:01:01:01, DRB1*04:01:01:02, DRB1*04:01:01:03, DRB1*04:01:02, DRB1*04:01:03, DRB1*04:01:04, DRB1*04:01:05, DRB1*04:01:06, DRB1*04:01:07, DRB1*04:01:08, DRB1*04:01:09, DRB1*04:01:10, DRB1*04:01:11, DRB1*04:01:12, DRB1*04:01:13, DRB1*04:01:14, DRB1*04:01:15, DRB1*04:01:16, DRB1*04:01:17, DRB1*04:01:18, DRB1*04:01:19, DRB1*04:01:20, DRB1*04:01:21, DRB1*04:02:01, DRB1*04:02:02, DRB1*04:02:03, DRB1*04:02:04, DRB1*04:02:05, DRB1*04:02:06, DRB1*04:03:01:01, DRB1*04:03:01:02, DRB1*04:03:02, DRB1*04:03:03, DRB1*04:03:04, DRB1*04:03:05, DRB1*04:03:06, DRB1*04:03:07, DRB1*04:03:08, DRB1*04:03:09, DRB1*04:03:10, DRB1*04:03:11, DRB1*04:03:12, DRB1*04:03:13, DRB1*04:03:14, DRB1*04:03:15, DRB1*04:04:01, DRB1*04:04:02, DRB1*04:04:03, DRB1*04:04:04, DRB1*04:04:05, DRB1*04:04:06, DRB1*04:04:07, DRB1*04:04:08, DRB1*04:04:09, DRB1*04:04:10, DRB1*04:04:11, DRB1*04:04:12, DRB1*04:04:13, DRB1*04:04:14, DRB1*04:04:15, DRB1*04:05:01:01, DRB1*04:05:01:02, DRB1*04:05:01:03, DRB1*04:05:02, DRB1*04:05:03, DRB1*04:05:04, DRB1*04:05:05, DRB1*04:05:06, DRB1*04:05:07, DRB1*04:05:08, DRB1*04:05:09, DRB1*04:05:10, DRB1*04:05:11, DRB1*04:05:13, DRB1*04:05:14, DRB1*04:05:15, DRB1*04:05:16, DRB1*04:05:17, DRB1*04:05:18, DRB1*04:05:19, DRB1*04:05:20, DRB1*04:06:01, DRB1*04:06:02, DRB1*04:06:03, DRB1*04:06:04, DRB1*04:06:05, DRB1*04:06:06, DRB1*04:06:07, DRB1*04:07:01:01, DRB1*04:07:01:02, DRB1*04:07:02, DRB1*04:07:03, DRB1*04:07:04, DRB1*04:07:05, DRB1*04:07:06, DRB1*04:08:01, DRB1*04:08:02, DRB1*04:08:03, DRB1*04:08:04, DRB1*04:09, DRB1*04:100, DRB1*04:101, DRB1*04:102, DRB1*04:103, DRB1*04:104, DRB1*04:105:01, DRB1*04:105:02, DRB1*04:106, DRB1*04:107, DRB1*04:108, DRB1*04:109, DRB1*04:10:01, DRB1*04:10:02, DRB1*04:10:03, DRB1*04:110, DRB1*04:111, DRB1*04:112, DRB1*04:113, DRB1*04:114, DRB1*04:115, DRB1*04:116, DRB1*04:117, DRB1*04:118, DRB1*04:119N, DRB1*04:11:01, DRB1*04:11:02, DRB1*04:11:03, DRB1*04:11:04, DRB1*04:11:05, DRB1*04:12, DRB1*04:120N, DRB1*04:121, DRB1*04:122, DRB1*04:123, DRB1*04:124, DRB1*04:125, DRB1*04:126, DRB1*04:127, DRB1*04:128, DRB1*04:129, DRB1*04:13, DRB1*04:130, DRB1*04:131:01, DRB1*04:131:02, DRB1*04:132, DRB1*04:133, DRB1*04:134, DRB1*04:135, DRB1*04:136, DRB1*04:137, DRB1*04:138, DRB1*04:139, DRB1*04:14, DRB1*04:140, DRB1*04:141, DRB1*04:142N, DRB1*04:143, DRB1*04:144, DRB1*04:145, DRB1*04:146, DRB1*04:147, DRB1*04:148, DRB1*04:149, DRB1*04:15, DRB1*04:150, DRB1*04:151, DRB1*04:152, DRB1*04:153, DRB1*04:154, DRB1*04:155, DRB1*04:156, DRB1*04:157N, DRB1*04:158N, DRB1*04:159, DRB1*04:16, DRB1*04:160, DRB1*04:161, DRB1*04:162, DRB1*04:163, DRB1*04:164, DRB1*04:165, DRB1*04:166, DRB1*04:167, DRB1*04:168, DRB1*04:169, DRB1*04:170, DRB1*04:171, DRB1*04:172, DRB1*04:173, DRB1*04:174, DRB1*04:175, DRB1*04:176, DRB1*04:177, DRB1*04:178N, DRB1*04:179, DRB1*04:17:01, DRB1*04:17:02, DRB1*04:18, DRB1*04:180, DRB1*04:181, DRB1*04:182, DRB1*04:183, DRB1*04:184, DRB1*04:185, DRB1*04:186N, DRB1*04:187, DRB1*04:188, DRB1*04:189, DRB1*04:19, DRB1*04:190, DRB1*04:191, DRB1*04:192, DRB1*04:193, DRB1*04:194, DRB1*04:195, DRB1*04:196, DRB1*04:197, DRB1*04:198, DRB1*04:199, DRB1*04:20, DRB1*04:200, DRB1*04:201, DRB1*04:202, DRB1*04:203, DRB1*04:204, DRB1*04:205, DRB1*04:206, DRB1*04:207, DRB1*04:208, DRB1*04:209, DRB1*04:21, DRB1*04:210, DRB1*04:211, DRB1*04:212N, DRB1*04:213, DRB1*04:214N, DRB1*04:215, DRB1*04:216, DRB1*04:217, DRB1*04:218, DRB1*04:219, DRB1*04:22, DRB1*04:220, DRB1*04:221, DRB1*04:222, DRB1*04:223, DRB1*04:224, DRB1*04:225, DRB1*04:226:01, DRB1*04:226:02, DRB1*04:227, DRB1*04:228, DRB1*04:229, DRB1*04:23, DRB1*04:230, DRB1*04:231, DRB1*04:232, DRB1*04:233, DRB1*04:234, DRB1*04:235, DRB1*04:236, DRB1*04:237, DRB1*04:238, DRB1*04:239, DRB1*04:24, DRB1*04:240, DRB1*04:241, DRB1*04:242, DRB1*04:243, DRB1*04:244, DRB1*04:245, DRB1*04:246, DRB1*04:247N, DRB1*04:248, DRB1*04:249, DRB1*04:25, DRB1*04:250, DRB1*04:251, DRB1*04:252, DRB1*04:253, DRB1*04:254, DRB1*04:255, DRB1*04:256, DRB1*04:257, DRB1*04:258, DRB1*04:259, DRB1*04:26, DRB1*04:260, DRB1*04:261, DRB1*04:262, DRB1*04:263, DRB1*04:264N, DRB1*04:265, DRB1*04:266N, DRB1*04:267N, DRB1*04:268, DRB1*04:269, DRB1*04:27, DRB1*04:270, DRB1*04:271, DRB1*04:272, DRB1*04:28, DRB1*04:29, DRB1*04:30, DRB1*04:31, DRB1*04:32, DRB1*04:33, DRB1*04:34, DRB1*04:35, DRB1*04:36, DRB1*04:37, DRB1*04:38, DRB1*04:39, DRB1*04:40, DRB1*04:41, DRB1*04:42, DRB1*04:43, DRB1*04:44:01, DRB1*04:44:02, DRB1*04:45, DRB1*04:46, DRB1*04:47, DRB1*04:48, DRB1*04:49, DRB1*04:50, DRB1*04:51, DRB1*04:52, DRB1*04:53:01, DRB1*04:53:02, DRB1*04:54, DRB1*04:55, DRB1*04:56:01, DRB1*04:56:02, DRB1*04:57, DRB1*04:58, DRB1*04:59, DRB1*04:60, DRB1*04:61, DRB1*04:62, DRB1*04:63, DRB1*04:64, DRB1*04:65, DRB1*04:66, DRB1*04:67, DRB1*04:68, DRB1*04:69, DRB1*04:70, DRB1*04:71, DRB1*04:72:01, DRB1*04:72:02, DRB1*04:73:01, DRB1*04:73:02, DRB1*04:74, DRB1*04:75, DRB1*04:76, DRB1*04:77, DRB1*04:78, DRB1*04:79, DRB1*04:80, DRB1*04:81N, DRB1*04:82, DRB1*04:83, DRB1*04:84, DRB1*04:85, DRB1*04:86, DRB1*04:87, DRB1*04:88, DRB1*04:89, DRB1*04:90, DRB1*04:91, DRB1*04:92, DRB1*04:93, DRB1*04:94:01N, DRB1*04:95:01, DRB1*04:95:02, DRB1*04:96, DRB1*04:97, DRB1*04:98:01, DRB1*04:98:02, DRB1*04:99, DRB1*07:01:01:01, DRB1*07:01:01:02, DRB1*07:01:01:03, DRB1*07:01:01:04, DRB1*07:01:02, DRB1*07:01:03, DRB1*07:01:04, DRB1*07:01:05, DRB1*07:01:06, DRB1*07:01:07, DRB1*07:01:08, DRB1*07:01:09, DRB1*07:01:10, DRB1*07:01:11, DRB1*07:01:12, DRB1*07:01:13, DRB1*07:01:14, DRB1*07:01:15, DRB1*07:01:16, DRB1*07:01:17, DRB1*07:01:18, DRB1*07:01:19, DRB1*07:01:20, DRB1*07:01:21, DRB1*07:01:22, DRB1*07:03, DRB1*07:04, DRB1*07:05, DRB1*07:06, DRB1*07:07, DRB1*07:08, DRB1*07:09, DRB1*07:100, DRB1*07:101N, DRB1*07:10N, DRB1*07:11, DRB1*07:12, DRB1*07:13, DRB1*07:14, DRB1*07:15, DRB1*07:16, DRB1*07:17, DRB1*07:18, DRB1*07:19, DRB1*07:20, DRB1*07:21, DRB1*07:22, DRB1*07:23, DRB1*07:24, DRB1*07:25, DRB1*07:26N, DRB1*07:27, DRB1*07:28, DRB1*07:29, DRB1*07:30, DRB1*07:31, DRB1*07:32, DRB1*07:33, DRB1*07:34, DRB1*07:35, DRB1*07:36, DRB1*07:37, DRB1*07:38, DRB1*07:39, DRB1*07:40, DRB1*07:41, DRB1*07:42, DRB1*07:43, DRB1*07:44, DRB1*07:45, DRB1*07:46, DRB1*07:47, DRB1*07:48, DRB1*07:49, DRB1*07:50, DRB1*07:51, DRB1*07:52, DRB1*07:53, DRB1*07:54, DRB1*07:55, DRB1*07:56, DRB1*07:57, DRB1*07:58N, DRB1*07:59, DRB1*07:60, DRB1*07:61, DRB1*07:62, DRB1*07:63, DRB1*07:64, DRB1*07:65, DRB1*07:66, DRB1*07:67, DRB1*07:68N, DRB1*07:69, DRB1*07:70, DRB1*07:71, DRB1*07:72, DRB1*07:73, DRB1*07:74, DRB1*07:75, DRB1*07:76, DRB1*07:77, DRB1*07:78, DRB1*07:79, DRB1*07:80, DRB1*07:81, DRB1*07:82, DRB1*07:83, DRB1*07:84, DRB1*07:85, DRB1*07:86, DRB1*07:87N, DRB1*07:88, DRB1*07:89, DRB1*07:90, DRB1*07:91, DRB1*07:92, DRB1*07:93, DRB1*07:94, DRB1*07:95, DRB1*07:96, DRB1*07:97, DRB1*07:98, DRB1*07:99, DRB1*08:01:01, DRB1*08:01:02, DRB1*08:01:04, DRB1*08:01:05, DRB1*08:01:06, DRB1*08:01:07, DRB1*08:02:01:01, DRB1*08:02:01:02, DRB1*08:02:02, DRB1*08:02:03, DRB1*08:02:04, DRB1*08:03:02:01, DRB1*08:03:02:02, DRB1*08:03:03, DRB1*08:03:04, DRB1*08:03:05, DRB1*08:03:06, DRB1*08:03:07, DRB1*08:03:08, DRB1*08:03:09, DRB1*08:04:01, DRB1*08:04:02, DRB1*08:04:03, DRB1*08:04:04, DRB1*08:04:05, DRB1*08:04:06, DRB1*08:04:07, DRB1*08:05, DRB1*08:06, DRB1*08:07, DRB1*08:08, DRB1*08:09, DRB1*08:10, DRB1*08:11, DRB1*08:12, DRB1*08:13, DRB1*08:14, DRB1*08:15, DRB1*08:16, DRB1*08:17, DRB1*08:18, DRB1*08:19, DRB1*08:20, DRB1*08:21, DRB1*08:22, DRB1*08:23, DRB1*08:24:01, DRB1*08:24:02, DRB1*08:25, DRB1*08:26, DRB1*08:27, DRB1*08:28, DRB1*08:29, DRB1*08:30:01, DRB1*08:30:02, DRB1*08:30:03, DRB1*08:31, DRB1*08:32, DRB1*08:33, DRB1*08:34, DRB1*08:35, DRB1*08:36:01, DRB1*08:36:02, DRB1*08:37, DRB1*08:38, DRB1*08:39, DRB1*08:40, DRB1*08:41, DRB1*08:42, DRB1*08:43, DRB1*08:44, DRB1*08:45:01, DRB1*08:45:02, DRB1*08:46, DRB1*08:47, DRB1*08:48, DRB1*08:49, DRB1*08:50, DRB1*08:51, DRB1*08:52, DRB1*08:53, DRB1*08:54, DRB1*08:55, DRB1*08:56, DRB1*08:57, DRB1*08:58, DRB1*08:59, DRB1*08:60N, DRB1*08:61, DRB1*08:62, DRB1*08:63, DRB1*08:64, DRB1*08:65, DRB1*08:66, DRB1*08:67, DRB1*08:68:01, DRB1*08:68:02, DRB1*08:69, DRB1*08:70, DRB1*08:71, DRB1*08:72, DRB1*08:73, DRB1*08:74, DRB1*08:75, DRB1*08:76, DRB1*08:77, DRB1*08:78N, DRB1*08:79, DRB1*08:80, DRB1*08:81, DRB1*08:82, DRB1*08:83, DRB1*08:84, DRB1*08:85, DRB1*08:86, DRB1*08:87, DRB1*08:88, DRB1*08:89N, DRB1*08:90, DRB1*09:01:02:01, DRB1*09:01:02:02, DRB1*09:01:03, DRB1*09:01:04, DRB1*09:01:05, DRB1*09:01:06, DRB1*09:01:07, DRB1*09:01:08, DRB1*09:01:09, DRB1*09:01:10, DRB1*09:01:11, DRB1*09:02:01, DRB1*09:02:02, DRB1*09:03, DRB1*09:04, DRB1*09:05, DRB1*09:06, DRB1*09:07, DRB1*09:08, DRB1*09:09, DRB1*09:10, DRB1*09:11, DRB1*09:12, DRB1*09:13, DRB1*09:14, DRB1*09:15, DRB1*09:16, DRB1*09:17, DRB1*09:18, DRB1*09:19, DRB1*09:20, DRB1*09:21, DRB1*09:22, DRB1*09:23, DRB1*09:24, DRB1*09:25, DRB1*09:26, DRB1*09:27, DRB1*09:28, DRB1*09:29, DRB1*09:30, DRB1*09:31, DRB1*09:32, DRB1*09:33, DRB1*09:34, DRB1*09:35, DRB1*09:36, DRB1*09:37N, DRB1*09:38, DRB1*09:39, DRB1*09:40, DRB1*10:01:01:01, DRB1*10:01:01:02, DRB1*10:01:01:03, DRB1*10:01:02, DRB1*10:01:03, DRB1*10:01:04, DRB1*10:01:05, DRB1*10:01:06, DRB1*10:01:07, DRB1*10:01:08, DRB1*10:01:09, DRB1*10:01:10, DRB1*10:01:11, DRB1*10:01:12, DRB1*10:02, DRB1*10:03, DRB1*10:04, DRB1*10:05, DRB1*10:06, DRB1*10:07, DRB1*10:08, DRB1*10:09, DRB1*10:10, DRB1*10:11, DRB1*10:12, DRB1*10:13, DRB1*10:14, DRB1*10:15, DRB1*10:16, DRB1*10:17, DRB1*10:18, DRB1*10:19, DRB1*10:20, DRB1*10:21, DRB1*10:22, DRB1*10:23, DRB1*10:24, DRB1*10:25, DRB1*10:26, DRB1*10:27, DRB1*10:28, DRB1*10:29, DRB1*10:30, DRB1*10:31, DRB1*10:32, DRB1*10:33, DRB1*11:01:01:01, DRB1*11:01:01:02, DRB1*11:01:01:03, DRB1*11:01:01:04, DRB1*11:01:02, DRB1*11:01:03, DRB1*11:01:04, DRB1*11:01:05, DRB1*11:01:06, DRB1*11:01:07, DRB1*11:01:08, DRB1*11:01:09, DRB1*11:01:10, DRB1*11:01:11, DRB1*11:01:12, DRB1*11:01:13, DRB1*11:01:14, DRB1*11:01:15, DRB1*11:01:16, DRB1*11:01:17, DRB1*11:01:18, DRB1*11:01:19, DRB1*11:01:20, DRB1*11:01:21, DRB1*11:01:22, DRB1*11:01:23, DRB1*11:01:24, DRB1*11:01:25, DRB1*11:01:26, DRB1*11:01:27, DRB1*11:01:28, DRB1*11:01:29, DRB1*11:01:30, DRB1*11:01:31, DRB1*11:01:32, DRB1*11:01:33, DRB1*11:02:01, DRB1*11:02:02, DRB1*11:02:03, DRB1*11:02:04, DRB1*11:02:05, DRB1*11:03:01, DRB1*11:03:02, DRB1*11:03:03, DRB1*11:03:04, DRB1*11:04:01, DRB1*11:04:02, DRB1*11:04:03, DRB1*11:04:04, DRB1*11:04:05, DRB1*11:04:06, DRB1*11:04:07, DRB1*11:04:08, DRB1*11:04:09, DRB1*11:04:10, DRB1*11:04:11, DRB1*11:04:12, DRB1*11:04:13, DRB1*11:04:14, DRB1*11:04:15, DRB1*11:04:16, DRB1*11:04:17, DRB1*11:04:18, DRB1*11:05, DRB1*11:06:01, DRB1*11:06:02, DRB1*11:06:03, DRB1*11:07:01, DRB1*11:07:02, DRB1*11:08:01, DRB1*11:08:02, DRB1*11:08:03, DRB1*11:09, DRB1*11:100, DRB1*11:101:01, DRB1*11:101:02, DRB1*11:102:01, DRB1*11:102:02, DRB1*11:103:01, DRB1*11:103:02, DRB1*11:104, DRB1*11:105, DRB1*11:106, DRB1*11:107, DRB1*11:108, DRB1*11:109, DRB1*11:10:01, DRB1*11:10:02, DRB1*11:110, DRB1*11:111, DRB1*11:112, DRB1*11:113, DRB1*11:114, DRB1*11:115, DRB1*11:116, DRB1*11:117:01, DRB1*11:117:02, DRB1*11:118, DRB1*11:119, DRB1*11:11:01, DRB1*11:11:03, DRB1*11:120, DRB1*11:121, DRB1*11:122, DRB1*11:123, DRB1*11:124:01, DRB1*11:124:02, DRB1*11:125, DRB1*11:126, DRB1*11:127, DRB1*11:128, DRB1*11:129, DRB1*11:12:01, DRB1*11:12:02, DRB1*11:12:03, DRB1*11:130, DRB1*11:131, DRB1*11:132, DRB1*11:133, DRB1*11:134, DRB1*11:135, DRB1*11:136, DRB1*11:137, DRB1*11:138, DRB1*11:139, DRB1*11:13:01, DRB1*11:13:02, DRB1*11:140, DRB1*11:141, DRB1*11:142, DRB1*11:143, DRB1*11:144, DRB1*11:145, DRB1*11:146, DRB1*11:147:01, DRB1*11:147:02, DRB1*11:148, DRB1*11:149, DRB1*11:14:01, DRB1*11:14:02, DRB1*11:15, DRB1*11:150, DRB1*11:151, DRB1*11:152, DRB1*11:153, DRB1*11:154, DRB1*11:155, DRB1*11:156, DRB1*11:157, DRB1*11:158, DRB1*11:159, DRB1*11:16, DRB1*11:160, DRB1*11:161, DRB1*11:162, DRB1*11:163, DRB1*11:164, DRB1*11:165:01, DRB1*11:165:02, DRB1*11:166, DRB1*11:167, DRB1*11:168, DRB1*11:169N, DRB1*11:17, DRB1*11:170, DRB1*11:171, DRB1*11:172, DRB1*11:173, DRB1*11:174, DRB1*11:175, DRB1*11:176, DRB1*11:177, DRB1*11:178, DRB1*11:179, DRB1*11:18, DRB1*11:180, DRB1*11:181, DRB1*11:182, DRB1*11:183, DRB1*11:184, DRB1*11:185, DRB1*11:186, DRB1*11:187, DRB1*11:188, DRB1*11:189, DRB1*11:190, DRB1*11:191, DRB1*11:192, DRB1*11:193:01, DRB1*11:193:02, DRB1*11:194, DRB1*11:195, DRB1*11:196, DRB1*11:197, DRB1*11:198, DRB1*11:199, DRB1*11:19:01, DRB1*11:19:02, DRB1*11:19:03, DRB1*11:20, DRB1*11:200, DRB1*11:201, DRB1*11:202, DRB1*11:203, DRB1*11:204, DRB1*11:205, DRB1*11:206, DRB1*11:207, DRB1*11:208, DRB1*11:209, DRB1*11:21, DRB1*11:210, DRB1*11:211, DRB1*11:212, DRB1*11:213, DRB1*11:214, DRB1*11:215, DRB1*11:216, DRB1*11:217N, DRB1*11:218, DRB1*11:219, DRB1*11:22, DRB1*11:220, DRB1*11:221, DRB1*11:222, DRB1*11:223, DRB1*11:224, DRB1*11:225, DRB1*11:226, DRB1*11:227, DRB1*11:228, DRB1*11:229, DRB1*11:230, DRB1*11:231, DRB1*11:232, DRB1*11:233, DRB1*11:234, DRB1*11:235, DRB1*11:236, DRB1*11:237, DRB1*11:238, DRB1*11:239, DRB1*11:23:01, DRB1*11:23:02, DRB1*11:240, DRB1*11:241, DRB1*11:242, DRB1*11:243, DRB1*11:244, DRB1*11:245, DRB1*11:246N, DRB1*11:247, DRB1*11:248Q, DRB1*11:249, DRB1*11:24:01, DRB1*11:24:02, DRB1*11:25, DRB1*11:250N, DRB1*11:251, DRB1*11:252, DRB1*11:253, DRB1*11:254, DRB1*11:26, DRB1*11:27:01, DRB1*11:27:02, DRB1*11:27:03, DRB1*11:28:01, DRB1*11:28:02, DRB1*11:29:01, DRB1*11:29:02, DRB1*11:30, DRB1*11:31, DRB1*11:32, DRB1*11:33, DRB1*11:34, DRB1*11:35, DRB1*11:36, DRB1*11:37:01, DRB1*11:37:02, DRB1*11:38, DRB1*11:39, DRB1*11:40, DRB1*11:41, DRB1*11:42:01, DRB1*11:42:02, DRB1*11:43, DRB1*11:44, DRB1*11:45, DRB1*11:46:01, DRB1*11:46:02, DRB1*11:47, DRB1*11:48, DRB1*11:49:01, DRB1*11:49:02, DRB1*11:50, DRB1*11:51, DRB1*11:52, DRB1*11:53, DRB1*11:54:01, DRB1*11:54:02, DRB1*11:55, DRB1*11:56, DRB1*11:57, DRB1*11:58:01, DRB1*11:58:02, DRB1*11:59, DRB1*11:60, DRB1*11:61, DRB1*11:62:01, DRB1*11:62:02, DRB1*11:63:01, DRB1*11:63:02, DRB1*11:64, DRB1*11:65:01, DRB1*11:65:02, DRB1*11:66:01, DRB1*11:66:02, DRB1*11:67, DRB1*11:68, DRB1*11:69, DRB1*11:70, DRB1*11:72, DRB1*11:73, DRB1*11:74:01, DRB1*11:74:02, DRB1*11:75, DRB1*11:76, DRB1*11:77, DRB1*11:78, DRB1*11:79, DRB1*11:80, DRB1*11:81, DRB1*11:82, DRB1*11:83, DRB1*11:84:01, DRB1*11:84:02, DRB1*11:84:03, DRB1*11:85, DRB1*11:86, DRB1*11:87, DRB1*11:88, DRB1*11:89, DRB1*11:90, DRB1*11:91, DRB1*11:92, DRB1*11:93, DRB1*11:94, DRB1*11:95, DRB1*11:96, DRB1*11:97, DRB1*11:98, DRB1*11:99, DRB1*12:01:01:01, DRB1*12:01:01:02, DRB1*12:01:01:03, DRB1*12:01:01:04, DRB1*12:01:01:05, DRB1*12:01:01:06, DRB1*12:01:02, DRB1*12:01:03, DRB1*12:01:04, DRB1*12:01:05, DRB1*12:01:06, DRB1*12:01:07, DRB1*12:01:08, DRB1*12:01:09, DRB1*12:02:01:01, DRB1*12:02:01:02, DRB1*12:02:01:03, DRB1*12:02:01:04, DRB1*12:02:02, DRB1*12:02:03, DRB1*12:02:04, DRB1*12:02:05, DRB1*12:02:06, DRB1*12:02:07, DRB1*12:02:08, DRB1*12:02:09, DRB1*12:03:02, DRB1*12:03:03, DRB1*12:04, DRB1*12:05, DRB1*12:06, DRB1*12:07, DRB1*12:08, DRB1*12:09, DRB1*12:10, DRB1*12:11, DRB1*12:12, DRB1*12:13, DRB1*12:14, DRB1*12:15, DRB1*12:16:01, DRB1*12:16:02, DRB1*12:16:03, DRB1*12:17, DRB1*12:18, DRB1*12:19, DRB1*12:20, DRB1*12:21, DRB1*12:22, DRB1*12:23, DRB1*12:24N, DRB1*12:25, DRB1*12:26, DRB1*12:27, DRB1*12:28, DRB1*12:29, DRB1*12:30, DRB1*12:31N, DRB1*12:32, DRB1*12:33, DRB1*12:34, DRB1*12:35, DRB1*12:36, DRB1*12:37, DRB1*12:38, DRB1*12:39, DRB1*12:40, DRB1*12:41, DRB1*12:42, DRB1*12:43, DRB1*12:44, DRB1*12:45, DRB1*12:46, DRB1*12:47, DRB1*12:48, DRB1*12:49, DRB1*12:50, DRB1*12:51, DRB1*12:52, DRB1*12:53, DRB1*12:54, DRB1*12:55, DRB1*12:56, DRB1*12:57, DRB1*12:58, DRB1*12:59, DRB1*12:60N, DRB1*12:61, DRB1*12:62, DRB1*12:63, DRB1*12:64, DRB1*12:65, DRB1*12:66, DRB1*12:67, DRB1*12:68, DRB1*12:69, DRB1*12:70, DRB1*12:71, DRB1*12:72N, DRB1*12:73, DRB1*12:74N, DRB1*12:75, DRB1*13:01:01:01, DRB1*13:01:01:02, DRB1*13:01:02, DRB1*13:01:03, DRB1*13:01:04, DRB1*13:01:05, DRB1*13:01:06, DRB1*13:01:07, DRB1*13:01:08, DRB1*13:01:09, DRB1*13:01:10, DRB1*13:01:11, DRB1*13:01:12, DRB1*13:01:13, DRB1*13:01:14, DRB1*13:01:15, DRB1*13:01:16, DRB1*13:01:17, DRB1*13:01:18, DRB1*13:01:19, DRB1*13:01:20, DRB1*13:01:21, DRB1*13:01:22, DRB1*13:01:23, DRB1*13:01:24, DRB1*13:01:25, DRB1*13:01:26, DRB1*13:02:01:01, DRB1*13:02:01:02, DRB1*13:02:01:03, DRB1*13:02:02, DRB1*13:02:03, DRB1*13:02:04, DRB1*13:02:05, DRB1*13:02:06, DRB1*13:02:07, DRB1*13:02:08, DRB1*13:02:09, DRB1*13:02:10, DRB1*13:02:11, DRB1*13:02:12, DRB1*13:02:13, DRB1*13:02:14, DRB1*13:02:15, DRB1*13:02:16, DRB1*13:02:17, DRB1*13:03:01, DRB1*13:03:02, DRB1*13:03:03, DRB1*13:03:04, DRB1*13:03:05, DRB1*13:03:06, DRB1*13:03:07, DRB1*13:03:08, DRB1*13:03:09, DRB1*13:04, DRB1*13:05:01, DRB1*13:05:02, DRB1*13:05:03, DRB1*13:06, DRB1*13:07:01, DRB1*13:07:02, DRB1*13:08, DRB1*13:09, DRB1*13:10, DRB1*13:100, DRB1*13:101, DRB1*13:102, DRB1*13:103, DRB1*13:104, DRB1*13:105, DRB1*13:106, DRB1*13:107, DRB1*13:108, DRB1*13:109, DRB1*13:110, DRB1*13:111, DRB1*13:112, DRB1*13:113N, DRB1*13:114, DRB1*13:115, DRB1*13:116, DRB1*13:117, DRB1*13:118, DRB1*13:119, DRB1*13:11:01, DRB1*13:11:02, DRB1*13:120, DRB1*13:121, DRB1*13:122, DRB1*13:123, DRB1*13:124, DRB1*13:125, DRB1*13:126, DRB1*13:127, DRB1*13:128, DRB1*13:129, DRB1*13:12:01, DRB1*13:12:02, DRB1*13:12:03, DRB1*13:12:04, DRB1*13:13, DRB1*13:130, DRB1*13:131, DRB1*13:132, DRB1*13:133, DRB1*13:134, DRB1*13:135, DRB1*13:136, DRB1*13:137N, DRB1*13:138, DRB1*13:139, DRB1*13:140, DRB1*13:141, DRB1*13:142N, DRB1*13:143, DRB1*13:144, DRB1*13:145, DRB1*13:146, DRB1*13:147, DRB1*13:148, DRB1*13:149, DRB1*13:14:01, DRB1*13:14:02, DRB1*13:14:03, DRB1*13:15, DRB1*13:150, DRB1*13:151, DRB1*13:152, DRB1*13:153, DRB1*13:154, DRB1*13:155, DRB1*13:156, DRB1*13:157, DRB1*13:158, DRB1*13:159, DRB1*13:16, DRB1*13:160, DRB1*13:161, DRB1*13:162, DRB1*13:163, DRB1*13:164, DRB1*13:165, DRB1*13:166, DRB1*13:167, DRB1*13:168, DRB1*13:169, DRB1*13:17, DRB1*13:170, DRB1*13:171:01, DRB1*13:171:02, DRB1*13:172, DRB1*13:173, DRB1*13:174, DRB1*13:175, DRB1*13:176, DRB1*13:177, DRB1*13:178, DRB1*13:179, DRB1*13:18, DRB1*13:180, DRB1*13:181, DRB1*13:182, DRB1*13:183, DRB1*13:184, DRB1*13:185N, DRB1*13:186, DRB1*13:187, DRB1*13:188, DRB1*13:189, DRB1*13:19, DRB1*13:190, DRB1*13:191, DRB1*13:192, DRB1*13:193, DRB1*13:194, DRB1*13:195, DRB1*13:196, DRB1*13:197, DRB1*13:198, DRB1*13:199, DRB1*13:20, DRB1*13:200N, DRB1*13:201, DRB1*13:202, DRB1*13:203, DRB1*13:204, DRB1*13:205, DRB1*13:206, DRB1*13:207, DRB1*13:208, DRB1*13:209, DRB1*13:210, DRB1*13:211, DRB1*13:212, DRB1*13:213, DRB1*13:214, DRB1*13:215, DRB1*13:216, DRB1*13:217, DRB1*13:218, DRB1*13:219, DRB1*13:21:01, DRB1*13:21:02, DRB1*13:220, DRB1*13:221, DRB1*13:222, DRB1*13:223, DRB1*13:224, DRB1*13:225, DRB1*13:226, DRB1*13:227, DRB1*13:228, DRB1*13:229, DRB1*13:22:01, DRB1*13:22:02, DRB1*13:230, DRB1*13:231, DRB1*13:232, DRB1*13:233, DRB1*13:234, DRB1*13:235, DRB1*13:236, DRB1*13:237, DRB1*13:238, DRB1*13:239, DRB1*13:23:01, DRB1*13:23:02, DRB1*13:24, DRB1*13:240, DRB1*13:241, DRB1*13:242:01, DRB1*13:242:02, DRB1*13:243, DRB1*13:244, DRB1*13:245, DRB1*13:246, DRB1*13:247, DRB1*13:248, DRB1*13:249N, DRB1*13:25, DRB1*13:250, DRB1*13:251, DRB1*13:252N, DRB1*13:253, DRB1*13:254, DRB1*13:255N, DRB1*13:256, DRB1*13:257, DRB1*13:258, DRB1*13:259, DRB1*13:260, DRB1*13:261, DRB1*13:262, DRB1*13:263, DRB1*13:264, DRB1*13:265, DRB1*13:266, DRB1*13:267, DRB1*13:268N, DRB1*13:269, DRB1*13:26:01, DRB1*13:26:02, DRB1*13:27, DRB1*13:270, DRB1*13:271, DRB1*13:272, DRB1*13:273, DRB1*13:274, DRB1*13:275, DRB1*13:276, DRB1*13:277, DRB1*13:278Q, DRB1*13:279, DRB1*13:28:01, DRB1*13:28:02, DRB1*13:29, DRB1*13:30, DRB1*13:31, DRB1*13:32, DRB1*13:33:01, DRB1*13:33:02, DRB1*13:33:03, DRB1*13:34, DRB1*13:35, DRB1*13:36, DRB1*13:37, DRB1*13:38, DRB1*13:39, DRB1*13:40, DRB1*13:41, DRB1*13:42, DRB1*13:43, DRB1*13:44, DRB1*13:45, DRB1*13:46, DRB1*13:47, DRB1*13:48, DRB1*13:49, DRB1*13:50:01, DRB1*13:50:02, DRB1*13:50:03, DRB1*13:51, DRB1*13:52, DRB1*13:53, DRB1*13:54, DRB1*13:55, DRB1*13:56, DRB1*13:57, DRB1*13:58, DRB1*13:59, DRB1*13:60, DRB1*13:61:01, DRB1*13:61:02, DRB1*13:62, DRB1*13:63, DRB1*13:64, DRB1*13:65, DRB1*13:66:01, DRB1*13:66:02, DRB1*13:67, DRB1*13:68, DRB1*13:69, DRB1*13:70, DRB1*13:71, DRB1*13:72, DRB1*13:73, DRB1*13:74, DRB1*13:75, DRB1*13:76, DRB1*13:77, DRB1*13:78, DRB1*13:79, DRB1*13:80, DRB1*13:81, DRB1*13:82, DRB1*13:83, DRB1*13:84, DRB1*13:85, DRB1*13:86, DRB1*13:87, DRB1*13:88, DRB1*13:89:01, DRB1*13:89:02, DRB1*13:90, DRB1*13:91, DRB1*13:92, DRB1*13:93, DRB1*13:94:01, DRB1*13:94:02, DRB1*13:95, DRB1*13:96:01, DRB1*13:96:02, DRB1*13:97:01, DRB1*13:97:02, DRB1*13:98, DRB1*13:99, DRB1*14:01:01, DRB1*14:01:02, DRB1*14:01:03, DRB1*14:01:04, DRB1*14:02:01:01, DRB1*14:02:01:02, DRB1*14:02:02, DRB1*14:02:03, DRB1*14:02:04, DRB1*14:02:05, DRB1*14:02:06, DRB1*14:02:07, DRB1*14:03:01, DRB1*14:03:02, DRB1*14:04:01, DRB1*14:04:02, DRB1*14:04:03, DRB1*14:04:04, DRB1*14:04:05, DRB1*14:04:06, DRB1*14:05:01:01, DRB1*14:05:01:02, DRB1*14:05:02, DRB1*14:05:03, DRB1*14:05:04, DRB1*14:06:01, DRB1*14:06:02, DRB1*14:06:03, DRB1*14:06:04, DRB1*14:07:01, DRB1*14:07:02, DRB1*14:08, DRB1*14:09, DRB1*14:10, DRB1*14:100, DRB1*14:101, DRB1*14:102, DRB1*14:103, DRB1*14:104, DRB1*14:105, DRB1*14:106, DRB1*14:107, DRB1*14:108, DRB1*14:109, DRB1*14:11, DRB1*14:110, DRB1*14:111, DRB1*14:112, DRB1*14:113, DRB1*14:114, DRB1*14:115, DRB1*14:116, DRB1*14:117, DRB1*14:118, DRB1*14:119, DRB1*14:120, DRB1*14:121, DRB1*14:122, DRB1*14:123, DRB1*14:124, DRB1*14:125, DRB1*14:126:01, DRB1*14:126:02, DRB1*14:127:01, DRB1*14:127:02, DRB1*14:128, DRB1*14:129, DRB1*14:12:01, DRB1*14:12:02, DRB1*14:13, DRB1*14:130, DRB1*14:131, DRB1*14:132, DRB1*14:133, DRB1*14:134, DRB1*14:135, DRB1*14:136, DRB1*14:137N, DRB1*14:138, DRB1*14:139, DRB1*14:14, DRB1*14:140, DRB1*14:141, DRB1*14:142, DRB1*14:143, DRB1*14:144, DRB1*14:145, DRB1*14:146, DRB1*14:147, DRB1*14:148, DRB1*14:149, DRB1*14:15, DRB1*14:150, DRB1*14:151, DRB1*14:152N, DRB1*14:153, DRB1*14:154, DRB1*14:155, DRB1*14:156, DRB1*14:157, DRB1*14:158, DRB1*14:159, DRB1*14:16, DRB1*14:160, DRB1*14:161, DRB1*14:162, DRB1*14:163, DRB1*14:164, DRB1*14:165, DRB1*14:166N, DRB1*14:167, DRB1*14:168, DRB1*14:169, DRB1*14:17, DRB1*14:170, DRB1*14:171, DRB1*14:172, DRB1*14:173, DRB1*14:174, DRB1*14:175, DRB1*14:176, DRB1*14:177, DRB1*14:178, DRB1*14:179, DRB1*14:18, DRB1*14:180, DRB1*14:181, DRB1*14:182, DRB1*14:183, DRB1*14:184, DRB1*14:185, DRB1*14:186, DRB1*14:187, DRB1*14:188N, DRB1*14:189, DRB1*14:19, DRB1*14:190, DRB1*14:191, DRB1*14:192, DRB1*14:193, DRB1*14:194, DRB1*14:195N, DRB1*14:196, DRB1*14:197N, DRB1*14:198, DRB1*14:199, DRB1*14:20, DRB1*14:200, DRB1*14:201, DRB1*14:202, DRB1*14:203, DRB1*14:204, DRB1*14:205, DRB1*14:206, DRB1*14:207, DRB1*14:208, DRB1*14:209, DRB1*14:21, DRB1*14:210Q, DRB1*14:211, DRB1*14:22, DRB1*14:23:01, DRB1*14:23:02, DRB1*14:23:03, DRB1*14:23:04, DRB1*14:24, DRB1*14:25:01, DRB1*14:25:02, DRB1*14:26, DRB1*14:27:01, DRB1*14:27:02, DRB1*14:28, DRB1*14:29, DRB1*14:30, DRB1*14:31, DRB1*14:32:01, DRB1*14:32:02, DRB1*14:32:03, DRB1*14:33, DRB1*14:34, DRB1*14:35, DRB1*14:36, DRB1*14:37, DRB1*14:38:01, DRB1*14:38:02, DRB1*14:39, DRB1*14:40, DRB1*14:41, DRB1*14:42, DRB1*14:43, DRB1*14:44:01, DRB1*14:44:02, DRB1*14:44:03, DRB1*14:45, DRB1*14:46, DRB1*14:47, DRB1*14:48, DRB1*14:49, DRB1*14:50, DRB1*14:51, DRB1*14:52, DRB1*14:53, DRB1*14:54:01:01, DRB1*14:54:01:02, DRB1*14:54:01:03, DRB1*14:54:01:04, DRB1*14:54:02, DRB1*14:54:03, DRB1*14:54:04, DRB1*14:54:05, DRB1*14:54:06, DRB1*14:54:07, DRB1*14:55, DRB1*14:56, DRB1*14:57, DRB1*14:58, DRB1*14:59, DRB1*14:60, DRB1*14:61, DRB1*14:62, DRB1*14:63, DRB1*14:64, DRB1*14:65, DRB1*14:67, DRB1*14:68:01, DRB1*14:68:02, DRB1*14:69, DRB1*14:70, DRB1*14:71, DRB1*14:72, DRB1*14:73, DRB1*14:74, DRB1*14:75, DRB1*14:76, DRB1*14:77, DRB1*14:78, DRB1*14:79, DRB1*14:80, DRB1*14:81, DRB1*14:82, DRB1*14:83, DRB1*14:84, DRB1*14:85, DRB1*14:86, DRB1*14:87, DRB1*14:88, DRB1*14:89, DRB1*14:90, DRB1*14:91, DRB1*14:92N, DRB1*14:93, DRB1*14:94, DRB1*14:95, DRB1*14:96, DRB1*14:97, DRB1*14:98, DRB1*14:99, DRB1*15:01:01:01, DRB1*15:01:01:02, DRB1*15:01:01:03, DRB1*15:01:01:04, DRB1*15:01:01:05, DRB1*15:01:02, DRB1*15:01:03, DRB1*15:01:04, DRB1*15:01:05, DRB1*15:01:06, DRB1*15:01:07, DRB1*15:01:08, DRB1*15:01:09, DRB1*15:01:10, DRB1*15:01:11, DRB1*15:01:12, DRB1*15:01:13, DRB1*15:01:14, DRB1*15:01:15, DRB1*15:01:16, DRB1*15:01:17, DRB1*15:01:18, DRB1*15:01:19, DRB1*15:01:20, DRB1*15:01:21, DRB1*15:01:22, DRB1*15:01:23, DRB1*15:01:24, DRB1*15:01:25, DRB1*15:01:26, DRB1*15:01:27, DRB1*15:01:28, DRB1*15:01:29, DRB1*15:01:30, DRB1*15:01:31, DRB1*15:01:32, DRB1*15:01:33, DRB1*15:01:34, DRB1*15:01:35, DRB1*15:01:36, DRB1*15:01:37, DRB1*15:01:38, DRB1*15:01:39, DRB1*15:01:40, DRB1*15:01:41, DRB1*15:02:01:01, DRB1*15:02:01:02, DRB1*15:02:01:03, DRB1*15:02:02, DRB1*15:02:03, DRB1*15:02:04, DRB1*15:02:05, DRB1*15:02:06, DRB1*15:02:07, DRB1*15:02:08, DRB1*15:02:09, DRB1*15:02:10, DRB1*15:02:11, DRB1*15:02:12, DRB1*15:02:13, DRB1*15:02:14, DRB1*15:02:15, DRB1*15:02:16, DRB1*15:02:17, DRB1*15:02:18, DRB1*15:02:19, DRB1*15:03:01:01, DRB1*15:03:01:02, DRB1*15:03:01:03, DRB1*15:03:02, DRB1*15:03:03, DRB1*15:03:04, DRB1*15:04, DRB1*15:05, DRB1*15:06:01, DRB1*15:06:02, DRB1*15:06:03, DRB1*15:06:04, DRB1*15:07:01, DRB1*15:07:02, DRB1*15:07:03, DRB1*15:08, DRB1*15:09, DRB1*15:10, DRB1*15:100, DRB1*15:101, DRB1*15:102, DRB1*15:103, DRB1*15:104:01, DRB1*15:104:02, DRB1*15:104:03, DRB1*15:105:01, DRB1*15:105:02, DRB1*15:106, DRB1*15:107, DRB1*15:108, DRB1*15:109, DRB1*15:110, DRB1*15:111, DRB1*15:112, DRB1*15:113N, DRB1*15:114, DRB1*15:115N, DRB1*15:116, DRB1*15:117, DRB1*15:118, DRB1*15:119, DRB1*15:11:01, DRB1*15:11:02, DRB1*15:12, DRB1*15:120, DRB1*15:121, DRB1*15:122, DRB1*15:123, DRB1*15:124, DRB1*15:125, DRB1*15:126, DRB1*15:127, DRB1*15:128, DRB1*15:129N, DRB1*15:13, DRB1*15:130, DRB1*15:131, DRB1*15:132, DRB1*15:133, DRB1*15:134N, DRB1*15:135, DRB1*15:136, DRB1*15:137N, DRB1*15:138N, DRB1*15:139, DRB1*15:14, DRB1*15:140, DRB1*15:141, DRB1*15:142, DRB1*15:143, DRB1*15:144, DRB1*15:145, DRB1*15:146, DRB1*15:147, DRB1*15:148N, DRB1*15:149, DRB1*15:150, DRB1*15:151, DRB1*15:152, DRB1*15:153, DRB1*15:154N, DRB1*15:155, DRB1*15:156, DRB1*15:157, DRB1*15:158, DRB1*15:159N, DRB1*15:15:01, DRB1*15:15:02, DRB1*15:15:03, DRB1*15:16, DRB1*15:160, DRB1*15:161, DRB1*15:162, DRB1*15:163N, DRB1*15:164Q, DRB1*15:165, DRB1*15:166, DRB1*15:167, DRB1*15:168, DRB1*15:169, DRB1*15:170, DRB1*15:17N, DRB1*15:18, DRB1*15:19, DRB1*15:20, DRB1*15:21, DRB1*15:22, DRB1*15:23, DRB1*15:24, DRB1*15:25, DRB1*15:26, DRB1*15:27, DRB1*15:28, DRB1*15:29, DRB1*15:30, DRB1*15:31:01, DRB1*15:31:02, DRB1*15:32, DRB1*15:33, DRB1*15:34, DRB1*15:35, DRB1*15:36, DRB1*15:37:01, DRB1*15:37:02, DRB1*15:38, DRB1*15:39, DRB1*15:40, DRB1*15:41, DRB1*15:42, DRB1*15:43, DRB1*15:44, DRB1*15:45, DRB1*15:46, DRB1*15:47, DRB1*15:48, DRB1*15:49, DRB1*15:50N, DRB1*15:51, DRB1*15:52, DRB1*15:53, DRB1*15:54, DRB1*15:55, DRB1*15:56, DRB1*15:57, DRB1*15:58, DRB1*15:59, DRB1*15:60, DRB1*15:61, DRB1*15:62, DRB1*15:63, DRB1*15:64, DRB1*15:65, DRB1*15:66:01, DRB1*15:66:02, DRB1*15:67, DRB1*15:68, DRB1*15:69, DRB1*15:70, DRB1*15:71, DRB1*15:72, DRB1*15:73, DRB1*15:74, DRB1*15:75, DRB1*15:76, DRB1*15:77, DRB1*15:78, DRB1*15:79, DRB1*15:80N, DRB1*15:81, DRB1*15:82, DRB1*15:83, DRB1*15:84, DRB1*15:85, DRB1*15:86, DRB1*15:87, DRB1*15:88, DRB1*15:89, DRB1*15:90, DRB1*15:91, DRB1*15:92, DRB1*15:93, DRB1*15:94, DRB1*15:95, DRB1*15:96, DRB1*15:97, DRB1*15:98, DRB1*15:99, DRB1*16:01:01, DRB1*16:01:02, DRB1*16:01:03, DRB1*16:01:04, DRB1*16:01:05, DRB1*16:01:06, DRB1*16:01:07, DRB1*16:01:08, DRB1*16:01:09, DRB1*16:01:10, DRB1*16:01:11, DRB1*16:01:12, DRB1*16:01:13, DRB1*16:01:14, DRB1*16:01:15, DRB1*16:01:16, DRB1*16:02:01:01, DRB1*16:02:01:02, DRB1*16:02:01:03, DRB1*16:02:02, DRB1*16:02:03, DRB1*16:02:04, DRB1*16:02:05, DRB1*16:02:06, DRB1*16:02:07, DRB1*16:02:08, DRB1*16:03, DRB1*16:04:01, DRB1*16:04:02, DRB1*16:05:01, DRB1*16:05:02, DRB1*16:07, DRB1*16:08, DRB1*16:09:01, DRB1*16:09:02, DRB1*16:10:01, DRB1*16:10:02, DRB1*16:11, DRB1*16:12, DRB1*16:13N, DRB1*16:14, DRB1*16:15, DRB1*16:16, DRB1*16:17, DRB1*16:18, DRB1*16:19, DRB1*16:20, DRB1*16:21N, DRB1*16:22, DRB1*16:23, DRB1*16:24, DRB1*16:25, DRB1*16:26, DRB1*16:27, DRB1*16:28, DRB1*16:29, DRB1*16:30, DRB1*16:31, DRB1*16:32, DRB1*16:33, DRB1*16:34, DRB1*16:35, DRB1*16:36, DRB1*16:37, DRB1*16:38:01, DRB1*16:38:02, DRB1*16:39, DRB1*16:40, DRB1*16:41N, DRB1*16:42, DRB1*16:43, DRB1*16:44, DRB1*16:45, DRB1*16:46, DRB1*16:47, DRB1*16:48, DRB1*16:49, DRB1*16:50, DRB1*16:51, DRB1*16:52, DRB1*16:53, DRB1*16:54, DRB1*16:55N, DRB1*16:56, and any combination thereof.

In certain aspects, the MHC class II molecule comprises a DR beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 3, wherein the DR beta chain comprises (i) a tryptophan at a position corresponding to amino acid residue 114 of SEQ ID NO: 1, (ii) a methionine at a position corresponding to amino acid residue 143 of SEQ ID NO: 1, (iii) a histidine at a position corresponding to amino acid residue 118 of SEQ ID NO: 1; and (iv) an isoleucine at a position corresponding to amino acid residue 157 of SEQ ID NO: 1. In certain aspects, the MHC class II molecule comprises a DR beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 3, wherein the DR beta chain comprises (i) a tryptophan at a position corresponding to amino acid residue 114 of SEQ ID NO: 1, (ii) a methionine at a position corresponding to amino acid residue 143 of SEQ ID NO: 1, (iii) a histidine at a position corresponding to amino acid residue 118 of SEQ ID NO: 1; (iv) an isoleucine at a position corresponding to amino acid residue 157 of SEQ ID NO: 1; (v) a threonine at a position corresponding to amino acid residue 139 of SEQ ID NO: 1; (vi) a glutamine at a position corresponding to amino acid residue 146 of SEQ ID NO: 1; (vii) a methionine at a position corresponding to amino acid residue 163 of SEQ ID NO: 1; and (viii) a threonine at a position corresponding to amino acid residue 164 of SEQ ID NO: 1. In certain aspects, the MHC class II molecule comprises a DR beta chain comprising an amino acid sequence set forth in SEQ ID NO: 3.

II.A.2. MHC Class II Alpha Chain

In some aspects of the present disclosure, the MHC class II molecule further comprises an alpha chain. In some aspects, the alpha chain is a wild-type alpha chain. In some aspects, the alpha chain is a DR alpha chain. Any DR alpha chain can be used in the compositions and methods of the present disclosure. In some aspects, the DR alpha chain comprises an HLA-DRA1*01 allele.

In certain aspects, the DR alpha chain is selected from DRA*01:01:01:01, DRA*01:01:01:02, DRA*01:01:01:03, DRA*01:01:02, DRA*01:02:01, DRA*01:02:02, DRA*01:02:03, and any combination thereof.

II.A.3. Signal Peptide

In some aspects, the DR beta chain and/or the DR alpha chain further comprises a signal peptide. Any signal peptide known in the art can be used in the compositions and methods disclosed herein. In some aspects the DR beta chain signal peptide is the same as the DR alpha signal peptide. In some aspects the DR beta chain signal peptide is different from the DR alpha signal peptide.

In some aspects, the signal peptide is derived from a native signal peptide. In some aspects, the signal peptide is derived from a naturally occurring DR beta chain signal peptide. In some aspects, the signal peptide comprises a naturally occurring DR beta chain signal peptide. In some aspects, the signal peptide is derived from a naturally occurring DR alpha chain signal peptide. In some aspects, the signal peptide comprises a naturally occurring DR alpha chain signal peptide. In some aspects, the signal peptide is derived from a fibroin light chain (FibL) signal peptide. In some aspects, the signal peptide comprises SEQ ID NO: 9. In some aspects, the signal peptide is synthetic.

II.A.4. Transmembrane Domain

In some aspects, the DR beta chain and/or the DR alpha chain further comprises a transmembrane domain. The transmembrane domain can be any length and of any origin. In some aspects, the transmembrane domain is at least about 1 to at least about 50 amino acid in length. In some aspects, the transmembrane domain is derived from a naturally occurring transmembrane domain. In some aspects, the transmembrane domain comprises a naturally occurring transmembrane domain. In some aspects, the transmembrane domain is derived from a naturally occurring HLA transmembrane domain. In some aspects, the transmembrane domain comprises a naturally occurring HLA transmembrane domain. In some aspects, the transmembrane domain is derived from a naturally occurring DR beta chain transmembrane domain. In some aspects, the transmembrane domain comprises a naturally occurring DR beta chain transmembrane domain. In some aspects, the transmembrane domain is derived from a naturally occurring DR alpha chain transmembrane domain. In some aspects, the transmembrane domain comprises a naturally occurring DR alpha chain transmembrane domain.

II.A.5. Leucine Zipper

In some aspects, the DR beta chain and/or the DR alpha chain further comprises one or more leucine zipper (LZip) sequences. Any LZip sequence known in the art can be used in the compositions and methods disclosed herein. In some aspects, the DR beta chain and/or the DR alpha chain comprises an acidic LZip (αLZip), a basic LZip (βLZip), or both. In some aspects, the one or more LZip sequences are derived from a naturally occurring LZip sequence. In some aspects, the one or more LZip sequences comprise a naturally occurring LZip sequence. In some aspects, the one or more LZip sequences are synthetic. In certain aspects, the one or more LZip sequences comprise the LZip sequences set forth in SEQ ID NO: 4 (Table 1).

II.A.6. Linker

In some aspects, the DR beta chain and/or the DR alpha chain useful for the disclosure further comprises a linker. Any linker known in the art can be used in the compositions and methods disclosed herein. In certain aspects, the linker comprises a Gly/Ser linker. In some aspects, the linker comprises an amino acid sequence selected from GlySer, Gly₂Ser, Gly₃Ser, and Gly₄Ser. In some aspects, the linker is positioned at the N-terminus of the extracellular domain of the DR alpha chain or the DR beta chain. In some aspects, the linker is positioned at the C-terminus of the extracellular domain of the DR alpha chain or the DR beta chain. In some aspects, the linker is positioned between the extracellular domain of the DR alpha chain or the DR beta chain and the transmembrane domain. In some aspects, the linker is positioned between the extracellular domain of the DR alpha chain or the DR beta chain and the one or more LZip sequences. In some aspects, the linker is positioned between the extracellular domain of the DR alpha chain or the DR beta chain and the signal peptide.

A linker of any length can be used in the compositions and methods disclosed herein. In some aspects, the linker is at least one amino acid in length. In some aspects, the linker is at least about 1 to at least about 100, at least about 1 to at least about 90, at least about 1 to at least about 80, at least about 1 to at least about 70, at least about 1 to at least about 60, at least about 1 to at least about 50, at least about 1 to at least about 40, at least about 1 to at least about 30, at least about 1 to at least about 20, at least about 1 to at least about 15, at least about 1 to at least about 14, at least about 1 to at least about 13, at least about 1 to at least about 12, at least about 1 to at least about 11, at least about 1 to at least about 10, at least about 1 to at least about 9, at least about 1 to at least about 8, at least about 1 to at least about 7, at least about 1 to at least about 6, at least about 1 to at least about 5, at least about 1 to at least about 4, at least about 1 to at least about 3 amino acids in length.

In some aspects, the linker is at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, at least about 50, at least about 60, at least about 70, at least about 80, at least about 90, at least about 100 amino acids in length. In certain aspects, the linker is about 3 amino acids in length. In certain aspects, the linker is about 4 amino acids in length. In certain aspects, the linker is about 5 amino acids in length.

II.B. Cells

In certain aspects of the present disclosure, the MHC class II molecule of the present disclosure is linked to or associated with a membrane of a cell. In certain aspects, the beta chain of the MHC class II molecule is linked or associated with a membrane of a cell. In certain aspects, the alpha chain of the MHC class II molecule is linked or associated with a membrane of a cell. In certain aspects, the alpha chain and the beta chain of the MHC class II molecule are linked or associated with a membrane of a cell.

Certain aspects of the present disclosure are directed to cells comprising an MHC class II molecule disclosed herein. Any cell can be used in the compositions described herein. In certain aspects the cell is a mammalian cell. In some aspects, the cell is an insect cell. In some aspects, the cell is derived from a healthy cell, e.g., a health fibroblast cell. In some aspects the cell is derived from a tumor cell. Non-limiting examples of cells that are useful in the present disclosure include K562 cells, T2 cells, HEK293 cells, HEK293T cells, A375 cells, SK-MEL-28 cells, Me275 cells, COS cells, fibroblast cells, tumor cells, or any combination thereof. In certain aspects, the cell is any cell disclosed in Hasan et al., Adv. Genet. Eng. 4(3):130 (2015), which is incorporated by reference herein in its entirety.

In certain aspects, the cell is a professional APC. In certain aspects, the cell is a macrophage, a B cell, a dendritic cell, or any combination thereof.

In certain aspects, the cell lacks endogenous expression of one or more MHC class II allele. In some aspects the cell lacks endogenous expression of an HLA-DR allele. In some aspects the cell lacks endogenous expression of an HLA-DR alpha chain allele. In some aspects the cell lacks endogenous expression of an HLA-DR beta chain allele.

II.C. Soluble MHC Class II Molecules

In certain aspects, the MHC class II molecule is not associated with a membrane of a cell, e.g., the MEW class II molecule is in a soluble form. As used herein, a soluble MEW class II molecule includes any MEW class II molecule or a portion thereof, described herein, that is not associated with a cell membrane. In certain aspects, the MEW class II molecule or portion thereof is unbound to any membrane. In some aspects, the MEW class II molecule or portion thereof is bound to an inert particle. In some aspects, the MEW class II molecule or portion thereof is bound to the membrane of an extracellular vesicle. In some aspects, the MHC class II molecule is bound to an artificial membrane or an artificial surface, e.g., the surface of an array plate.

Any inert particle known in the art can be used in the compositions and methods of the present disclosure. In some aspects, the inert particle is a bead. In some aspects, the bead is a glass bead, a latex bead, a metal bead, or any combination thereof. In some aspects, the inert particle is a nanoparticle (NP). Any NP known in the art can be used in the compositions and methods of the present disclosure. In certain aspects, the nanoparticle is selected from a pegylated iron oxide, chitosan, dextrane, gelatin, alginate, liposome, starch, branched polymer, carbon-based carrier, polylactic acid, poly(cyano)acrylate, polyethyleinemine, block copolymer, polycaprolactone, SPIONS, USPIONS, Cd/Zn-selenide, or silica nanoparticle. In particular aspects, the nanoparticle is a pegylated iron oxide nanoparticle. Nonlimiting examples of nanoparticles useful in the compositions and methods disclosed herein include those set forth in De Jong and Borm, Int. J. Nanomedicine 3(2):133-49 (2008) and Umeshappa et al., Nat. Commun. 10(1):2150 (May 14, 2019), each of which is incorporated by reference herein in its entirety.

In some aspects, the MEW class II molecule comprises a fragment of a full length MHC class II molecule, wherein one or more amino acids of the transmembrane domain of the alpha chain and/or the transmembrane domain of the beta chain are deleted. In some aspects, the MEW class II molecule comprises the extracellular domain of the alpha chain (e.g., as set forth in SEQ ID NO: 6) and/or the extracellular domain of the beta chain (e.g., as set forth in SEQ ID NO: 1 or 3). In certain aspects, the MHC class II molecule comprises a DR alpha chain comprising an amino acid sequence having at least about 80%, at least about 85%, at least about 90%, at least about 95%, at least about 96%, at least about 97%, at least about 98%, or at least about 99% sequence identity to SEQ ID NO: 6. In some aspects, the MHC class II molecule comprises a DR alpha chain comprising an amino acid sequence set forth in SEQ ID NO: 6.

In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 1. In some aspects, the MHC class II molecule comprises a DR beta chain comprising an amino acid sequence set forth in SEQ ID NO: 1. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 3. In some aspects, the MHC class II molecule comprises a DR beta chain comprising an amino acid sequence set forth in SEQ ID NO: 3. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 4. In some aspects, the MHC class II molecule comprises a DR beta chain comprising an amino acid sequence set forth in SEQ ID NO: 4. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 5. In some aspects, the MHC class II molecule comprises a DR beta chain comprising an amino acid sequence set forth in SEQ ID NO: 5.

II.D. Nucleic Acid Molecules and Vectors

Certain aspects of the present disclosure are directed to a nucleic acid molecule encoding an MHC class II molecule disclosed herein. In some aspects the nucleic acid molecule encodes an MHC class II beta chain disclosed herein. In certain aspects, the nucleic acid molecule encoding the MHC class II beta chain comprises a nucleotide sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the sequence set forth in SEQ ID NO: 2.

In some aspects the nucleic acid molecule encodes an MHC class II alpha chain disclosed herein. In certain aspects, the nucleic acid molecule encoding the MHC class II alpha chain comprises a nucleotide sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the sequence set forth in SEQ ID NO: 7.

In some aspects, the nucleic acid molecule encodes both an MHC class II alpha chain disclosed herein and an MHC class II beta chain disclosed herein. In some aspects, the sequence encoding the MHC class II alpha chain is under the control of the same promoter as the sequence encoding the MHC class II beta chain. In some aspects, the sequence encoding the MHC class II alpha chain is under the control of a first promoter, and the sequence encoding the MHC class II beta chain is under the control of a second promoter.

In some aspects, the present disclosure is directed to a first nucleic acid molecule encoding an MHC class II beta chain disclosed herein and a second nucleic acid molecule encoding an MHC class II alpha chain disclosed herein.

Certain aspects of the present disclosure are directed to a vector or a set of vectors comprising a nucleic acid molecule disclosed herein. In some aspects, the vector is a viral vector. In some aspects, the vector is a viral particle or a virus. In some aspects, the vector is a mammalian vector. In some aspects, the vector is a bacterial vector.

In certain aspects, the vector is a retroviral vector. In some aspects, the vector is an adenoviral vector, a lentivirus, a Sendai virus, a baculoviral vector, an Epstein Barr viral vector, a papovaviral vector, a vaccinia viral vector, a herpes simplex viral vector, or an adeno associated virus (AAV) vector. In particular aspects, the vector is an AAV vector. In some aspects, the vector is a lentivirus. In particular aspects, the vector is an adenoviral vector. In some aspects, the vector is a Sendai virus. In some aspects, the vector is a hybrid vector. Examples of hybrid vectors that can be used in the present disclosure can be found in Huang and Kamihira, Biotechnol. Adv. 31(2):208-23 (2103), which is incorporated by reference herein in its entirety.

### III. Methods of the Disclosure

Certain aspects of the present disclosure are directed to methods of treating a disease or condition in a subject. In some aspects, the disclosure is directed to methods of enhancing an immune response in a subject in need thereof.

III.A. Methods of Treating a Tumor

Certain aspects of the present disclosure are directed to methods of treating a cancer in a subject in need thereof, comprising administering to the subject an HLA class II molecule disclosed herein, a nucleic acid molecule disclosed herein, a vector disclosed herein, or a cell disclosed herein.

In some aspects, the cancer is selected from melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of the cancers. In some aspects, the cancer is melanoma.

In some aspects, the cancer is relapsed. In some aspects, the cancer is refractory. In some aspects, the cancer is advanced. In some aspects, the cancer is metastatic.

In some aspects, the methods disclosed herein treat a cancer in a subject. In some aspects, the methods disclosed herein reduce the severity of one or more symptom of the cancer. In some aspects, the methods disclosed herein reduce the size or number of a tumor derived from the cancer. In some aspects, the methods disclosed herein increase the overall survival of the subject, relative to a subject not provided the methods disclosed herein. In some aspects, the methods disclosed herein increase the progressive-free survival of the subject, relative to a subject not provided the methods disclosed herein. In some aspects, the methods disclosed herein lead to a partial response in the subject. In some aspects, the methods disclosed herein lead to a complete response in the subject.

Certain aspects of the present disclosure are directed to methods of treating an infection in a subject in need thereof, comprising administering to the subject an HLA class II molecule disclosed herein, a nucleic acid molecule disclosed herein, a vector disclosed herein, or a cell disclosed herein. Non-limiting examples of infections that can be treated using the compositions and methods disclosed herein include infection by a virus (including viroids and prions), a bacterium, a fungus, a parasite, or any combination thereof. In some aspects, the virus is herpesvirus, HIV, papvavirus, measles virus, rubella virus, human papillomavirus (HPV), human T-lymphotropic virus 1, Epstein-Barr virus, hepatitis A virus, hepatitis B virus, hepatitis C virus, influenza virus, norovirus, and any combination thereof. In some aspects, the bacterium is selected from Streptococcus, Staphylococcus, and E. coli. In some aspects, the bacterial infection is selected from Brucellosis, Campylobacter infections, Cat-scratch disease, Cholera, Escherichia coli, Gonorrhea, Klebsiella, Enterobacter, Serratia, Legionella infections, Meningococcal infection, Pertussis, Plague, Pseudomonas infection, Salmonella infection, Shigellosis, Typhoid fever, Tularemia, Anthrax, Diphtheria, Enterococcal infection, Erysipelothricosis, Listeriosis, Nocardiosis, Pneumococcal infection, Staphylococcal infection, Streptococcal infection, and any combination thereof. In some embodiments, the parasite infection is selected from pinworm, trichomononiasis, toxoplasmosis, giardiasis, cryptosporidiosis, malaria, hookwork, ringworm, tapeworm, fluke, and any combination thereof. In some aspects, the fungal infection is selected from Candida, Malassezia furfur, dermatophytes (e.g., Epidermophyton, Microsporum, and Trichophyton), or any combination thereof.

In some aspects, the methods disclosed herein comprise treating a cancer or an infection in a subject in need thereof, comprising administering to the subject a cell described herein, wherein the cell comprises an MHC class II molecule disclosed herein, a nucleic acid molecule disclosed herein, a vector disclosed herein, or any combination thereof.

In some aspects, the cell is obtained from the subject. In some aspects, the cell is obtained from a donor other than the subject.

III.B. Methods of Enriching a Target Population of T Cells

Certain aspects of the present disclosure are directed to methods of enriching a target population of T cells obtained from a human subject. In some aspects, the method comprises contacting the T cells with an HLA class II molecule disclosed herein. In some aspects, the method comprises contacting the T cells with a cell, e.g., an APC, disclosed herein. In some aspects, following the contacting, the enriched population of T cells comprises a higher number of T cells capable of binding the HLA class II molecule relative to the number of T cells capable of binding the HLA class II molecule prior to the contacting.

Some aspects of the present disclosure are directed to a method of selecting a T cell capable of targeting a diseased cell, e.g., tumor cell. In some aspects, the method comprises contacting a population of isolated T cells in vitro with a complex comprising an MHC class II molecule disclosed herein and a fragment of a polypeptide, e.g. an antigen expressed by a diseased cell, e.g., a tumor-expressed polypeptide, e.g., an epitope. In some aspects, the T cells are obtained from a human subject.

The T cells obtained from the human subject can be any T cells disclosed herein. In some aspects, the T cells obtained from the human subject are tumor infiltrating lymphocytes (TIL).

In some aspects, the method further comprises administering to the human subject the enriched T cells. In some aspects, the subject is preconditioned prior to receiving the T cells, as described herein.

All of the various aspects, aspects, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1—Methods

Cells

Peripheral mononuclear cells were obtained via density gradient centrifugation (Ficoll-Paque PLUS, GE Healthcare Life Sciences, Marlborough, Mass.). The K562 cell line is an erythroleukemic cell line with defective HLA class I/II expression. K562-based artificial APCs (aAPCs) individually expressing various HLA class II genes as a single HLA allele in conjunction with CD80 and CD83 have been reported previously (Butler et al., PloS One 7, e30229 (2012). HEK293T cells were grown in DMEM supplemented with 10% FBS and 50 μg/ml gentamicin (Thermo Fisher Scientific, Waltham, Mass.). The K562 cells were cultured in RPMI 1640 supplemented with 10% FBS and 50 μg/ml gentamicin.

Peptides

Synthetic peptides were purchased from Genscript (Piscataway, N.J.) and dissolved at 50 μg/ml in DMSO.

Antibodies

The following antibodies were used for flow cytometry analysis: PE-conjugated anti-class II (9-49 (I3)), APC-Cy7-conjugated anti-CD4 (RPA-T4, Biolegend, San Diego, Calif.)⁴⁴, PE-conjugated anti-His tag (AD1.1.10, Abcam, Cambridge, Mass.), and FITC-conjugated anti-Vβ22 (IMMU 546, Beckman Coulter, Brea, Calif.). Dead cells were distinguished with the LIVE/DEAD Fixable Near-IR Dead Cell Stain Kit 465 (Thermo Fisher Scientific, Waltham, Mass.). Stained cells were analyzed with Canto II or LSRFortessa X-20 (BD Biosciences, Franklin Lakes, N.J.). Cell sorting was conducted using a FACS Aria II (BD Biosciences, Franklin Lakes, N.J.). Data analysis was performed using FlowJo software (Tree Star, Ashland, Oreg.).

TCR Transduction into Primary T Cells

CD4⁺ T cells were purified using the CD4⁺ T Cell Isolation Kit (Miltenyi Biotec, Bergisch Gladbach, Germany). Purified T cells were stimulated with aAPC/mOKT3 irradiated with 200 Gy at an E:T ratio of 20:1. Starting the following day, activated T cells were retrovirally transduced with the cloned TCR genes via centrifugation for 1 hour at 1,000×g at 32° C. for 3 consecutive days or using a Retronectin-coated plate (Takara Bio, Shiga, Japan). On the following day, 100 IU/ml IL-2 and 10 ng/ml IL-15 were added to the TCR-transduced T cells. The culture medium was replenished every 2-3 days.

Staining with Soluble CD4

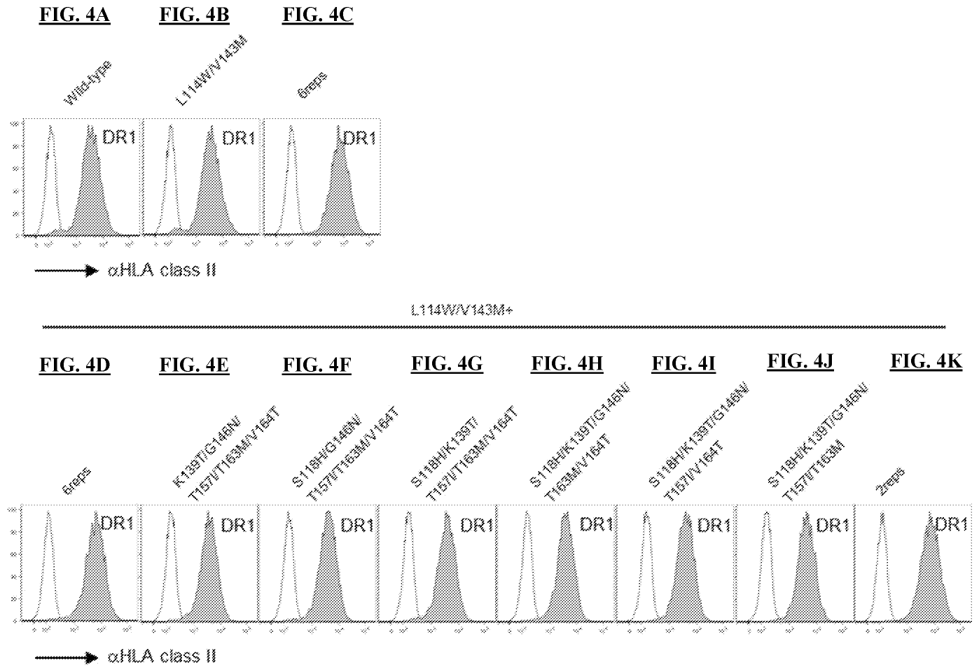

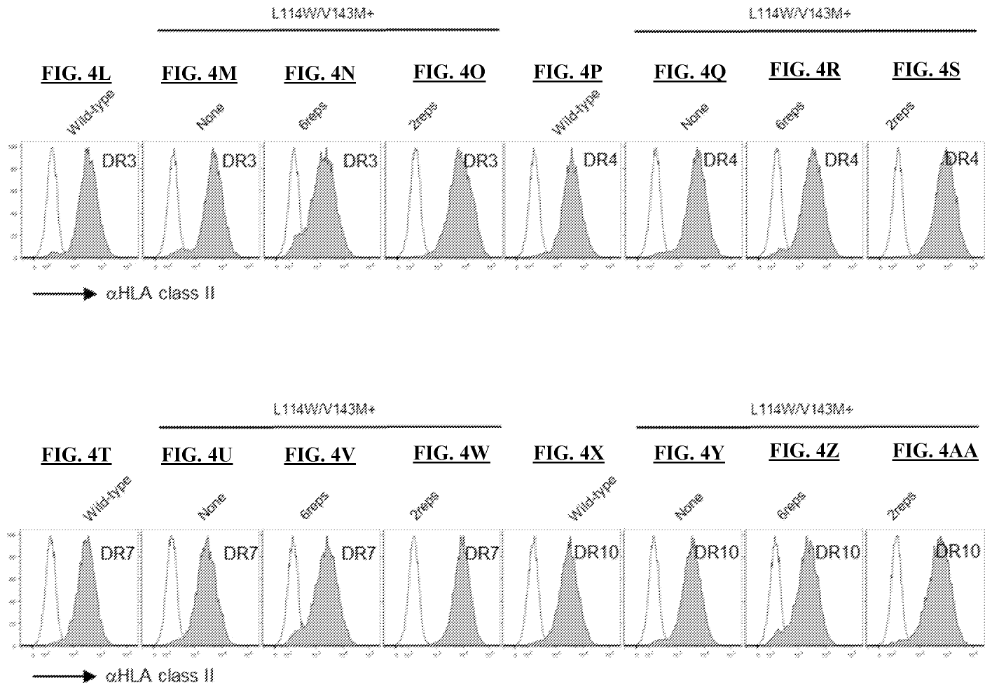

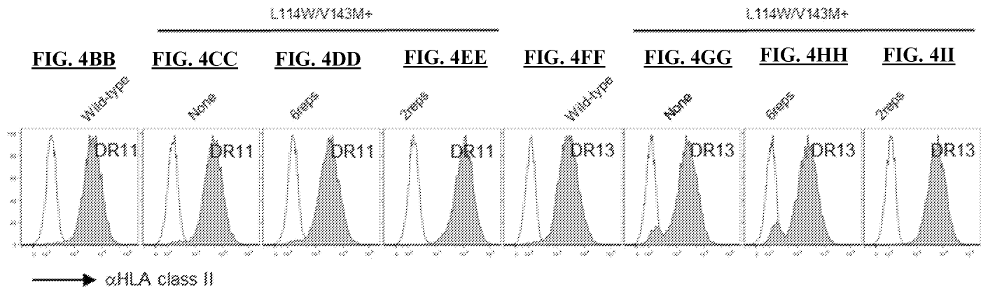

The soluble CD4 (sCD4) gene was generated by fusing the human CD4 extracellular domain with a 6×His tag via a GS linker. HEK293T cells were retrovirally transduced with the sCD4 gene, and the culture supernatant containing the sCD4 monomer was harvested. sCD4 was dimerized with a PE-labeled anti-6×His tag mAb (AD1.1.10, Abcam, Cambridge, Mass.) and used. HLA class II-expressing K562 cells were stained with dimerized sCD4 in the presence of goat serum for 30 min at room temperature. The surface HLA class II expression in K562-derived cells individually expressing various class II genes was as demonstrated in FIGS. 4A-4II.

Generation of the HLA Class II Monomer and Dimer

The extracellular domain of the wild-type class II α gene was fused with an acidic leucine zipper via a GGGS linker followed by a 6×His tag via a GS linker (see SEQ ID NO: 8). The ectodomain of the class II β gene carrying mutations (see SEQ ID NO: 3) was similarly linked with a basic leucine zipper via a GGGS linker (see SEQ ID NO: 4). HEK293T cells were transfected with the α and β genes using the 293GPG cell-based retrovirus system and cultured in DMEM supplemented with 10% FBS and 50 μg/ml gentamicin. For DR1 dimer staining, HEK293T cells stably secreting soluble DR1^{L114W/V143M+2reps}, DR7^{L114W/V143M+2reps} and DR11^{L114W/V143M+2reps} (which possesses the S118H/T157I replacements (2reps) in addition to L114W/V143M) protein were grown until confluent, and the after forty-eight hours, the medium was harvested. The soluble HLA class II-containing supernatant was then mixed with 100 μg/ml peptide of interest for 20-24 hours at 37° C. for in vitro peptide exchange. Monomer that was not subjected to peptide exchange was used as a control. The concentration of the monomer was measured by specific ELISA using a nickel-coated plate (XPressBio, Frederick, Md.) and an anti-His tag biotinylated mAb (AD1.1.10, R&D Systems, Minneapolis, Minn.). Soluble HLA class II monomer was dimerized using PE-conjugated anti-His mAb (AD1.1.10, Abcam, Cambridge, Mass.) at a 2:1 molar ratio for 1.5 hours at 4° C. for staining.

HLA Class II Dimer Staining

Primary T cells transduced with exogenous TCR gene were pretreated with 50 nM dasatinib (LC Laboratories, Woburn, Mass.) for 30 min at 37° C.⁴⁶ and stained with 5-15 μg/ml class II dimer for 4-5 hours at room temperature. After washing, cell surface molecules were counterstained with an APC-Cy7-conjugated anti-CD4 mAb and a PE-conjugated anti-Vβ22 mAb.

Protein Modeling

The HLA-DR1 and human CD4 complex model structures were predicted based on structures from PDB IDs 3S5L and 3T0E using Swiss-Model workspace for quaternary structure prediction.

Statistical Analysis

Statistical analysis was performed using GraphPad Prism 6.0 software (GraphPad Software, San Diego, Calif.). Unpaired two-tailed Student's t-tests were used for two-sample comparisons. No statistical method was used to predetermine sample size. The investigators were not blinded to allocation during the experiments or outcome assessment. The experiments were not randomized.

Biolayer Interferometry and Steady-State Analysis

The extracellular domain of human CD4 (residues 26-440 of NP_000607.1) followed by a GS linker and 10× histidine (His) tag was stably expressed in the human cell line A375 (SEQ ID NOs: 11-12). Recombinant 10× His-tagged CD4 protein was purified from the supernatant with TALON metal affinity resin (Takara Bio, Shiga, Japan). The eluted protein was concentrated using an Amicon Ultra-15 spin column (MilliporeSigma, Burlington, Mass.) with a 10 kDa MWCO. Buffer was exchanged to HBS-EP (GE Healthcare Life Sciences, Marlborough, Mass.) using 10 kDa MWCO MINI Dialyzer (Thermo Fisher Scientific, Waltham, Mass.). The purity of the recombinant CD4 protein was consistently >90%, as confirmed by SDS-PAGE.

The recombinant DR1 protein included extracellular domains of DRA1*01:01, and the wild-type DRB1*01:01 or L114W/V143M+2reps mutant. DRA1*01:01 was followed by an acid leucine zipper, a GS linker and a 10× histidine tag, while wild-type and mutant DRB1 was followed by a basic leucine zipper, a GS linker, and a biotinylation sequence (GLNDIFEAQKIEWHE; SEQ ID NO: 13). Both DRA and DRB genes were stably expressed in A375-BirA cells, which were transduced with the codon-optimized BirA gene encoding a leader sequence at the 5′ end and an ER retention KDEL motif at the 3′ end. Recombinant DR1 protein was purified from the supernatant with TALON metal affinity resin (Takara Bio, Shiga, Japan). Eluted protein was concentrated using Vivaspin 500 spin column (GE Healthcare Life Sciences, Marlborough, Mass.) with a 10 kDa MWCO, and reconstituted to working volume in PBS.

Binding for wild-type DR1 and DR1^{L114W/V143M+2reps} with CD4 was measured by the Octet Red system (ForteBio, Fremont, Calif.). Experiments were performed at 25° C. using a 96-well OptiPlate (Perkin Elmer, Waltham, Mass.), with a 200-μl sample volume and constant shaking at 1,000 rpm. The biotinylated recombinant DR1 was loaded onto streptavidin-coated biosensors (ForteBio, Fremont, Calif.) until saturation, followed by baseline measurement in the HBS-EP buffer. Association was measured by incubating the loaded sensors for 400 sec with titrated concentrations of recombinant CD4 (0.8125 to 26 μM) before 300 sec dissociation in HBS-EP buffer alone. The steady-state analysis was fitted using a one-site specific binding model in GraphPad Prism 7.0.

TABLE 3
Soluble 10x His-tagged CD4
Nucleic Acid Sequence
Fibroin L Signal Peptide; CD4; Gly/Ser Linker
and His tag sequences (10X))(SEQ ID NO: 11)
ATGATGCGGCCCATCGTGCTGGTGCTGCTGTTTGCCACATCTGCCCTG
GCCAAGAAAGTGGTGCTGGGCAAAAAAGGGGATACAGTGGAACTGACC
TGTACAGCTTCCCAGAAGAAGAGCATACAATTCCACTGGAAAAACTCC
AACCAGATAAAGATTCTGGGAAATCAGGGCTCCTTCTTAACTAAAGGT
CCATCCAAGCTGAATGATCGCGCTGACTCAAGAAGAAGCCTTTGGGAC
CAAGGAAACTTTCCCCTGATCATCAAGAATCTTAAGATAGAAGACTCA
GATACTTACATCTGTGAAGTGGAGGACCAGAAGGAGGAGGTGCAATTG
CTAGTGTTCGGATTGACTGCCAACTCTGACACCCACCTGCTTCAGGGG
CAGAGCCTGACCCTGACCTTGGAGAGCCCCCCTGGTAGTAGCCCCTCA
GTGCAATGTAGGAGTCCAAGGGGTAAAAACATACAGGGGGGGAAGACC
CTCTCCGTGTCTCAGCTGGAGCTCCAGGATAGTGGCACCTGGACATGC
ACTGTCTTGCAGAACCAGAAGAAGGTGGAGTTCAAAATAGACATCGTG
GTGCTAGCTTTCCAGAAGGCCTCCAGCATAGTCTATAAGAAAGAGGGG
GAACAGGTGGAGTTCTCCTTCCCACTCGCCTTTACAGTTGAAAAGCTG
ACGGGCAGTGGCGAGCTGTGGTGGCAGGCGGAGAGGGCTTCCTCCTCC
AAGTCTTGGATCACCTTTGACCTGAAGAACAAGGAAGTGTCTGTAAAA
CGGGTTACCCAGGACCCTAAGCTCCAGATGGGCAAGAAGCTCCCGCTC
CACCTCACCCTGCCCCAGGCCTTGCCTCAGTATGCTGGCTCTGGAAAC
CTCACCCTGGCCCTTGAAGCGAAAACAGGAAAGTTGCATCAGGAAGTG
AACCTGGTGGTGATGAGAGCCACTCAGCTCCAGAAAAATTTGACCTGT
GAGGTGTGGGGACCCACCTCCCCTAAGCTGATGCTGAGCTTGAAACTG
GAGAACAAGGAGGCAAAGGTCTCGAAGCGGGAGAAGGCGGTGTGGGTG
CTGAACCCTGAGGCGGGGATGTGGCAGTGTCTGCTGAGTGACTCGGGA
CAGGTCCTGCTGGAATCCAACATCAAGGTTCTGCCCACATGGGGCAGC
CACCACCACCATCACCATCATCATCACCATTGA
Fibroin L Signal Peptide; CD4; Gly/Ser Linker
and His tag sequences (10X))(SEQ ID NO: 12)
MMRPIVLVLLFATSALAKKVVLGKKGDTVELTCTASQKKSIQFHWKNS
NQIKILGNQGSFLTKGPSKLNDRADSRRSLWDQGNFPLIIKNLKIEDS
DTYICEVEDQKEEVQLLVFGLTANSDTHLLQGQSLTLTLESPPGSSPS
VQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSS
KSWITFDLKNKEVSVKRVTQDPKLQMGKKLPLHLTLPQALPQYAGSGN
LTLALEAKTGKLHQEVNLVVMRATQLQKNLTCEVWGPTSPKLMLSLKL
ENKEAKVSKREKAVWVLNPEAGMWQCLLSDSGQVLLESNIKVLPTWGS
HHHHHHHHHH

### Example 2—DR Molecules with Enhanced CD4 Binding Capacities

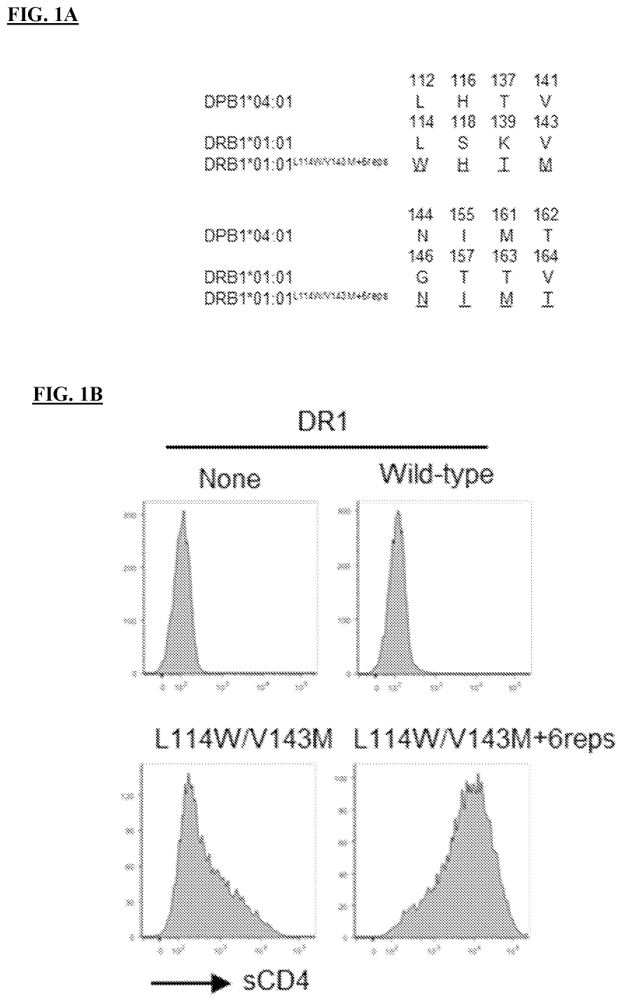

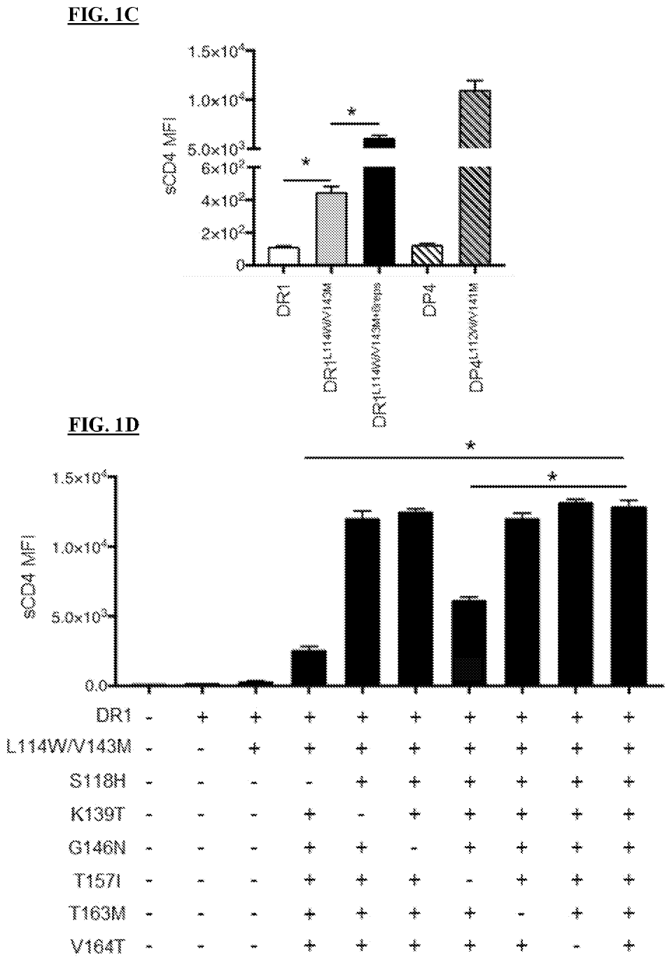

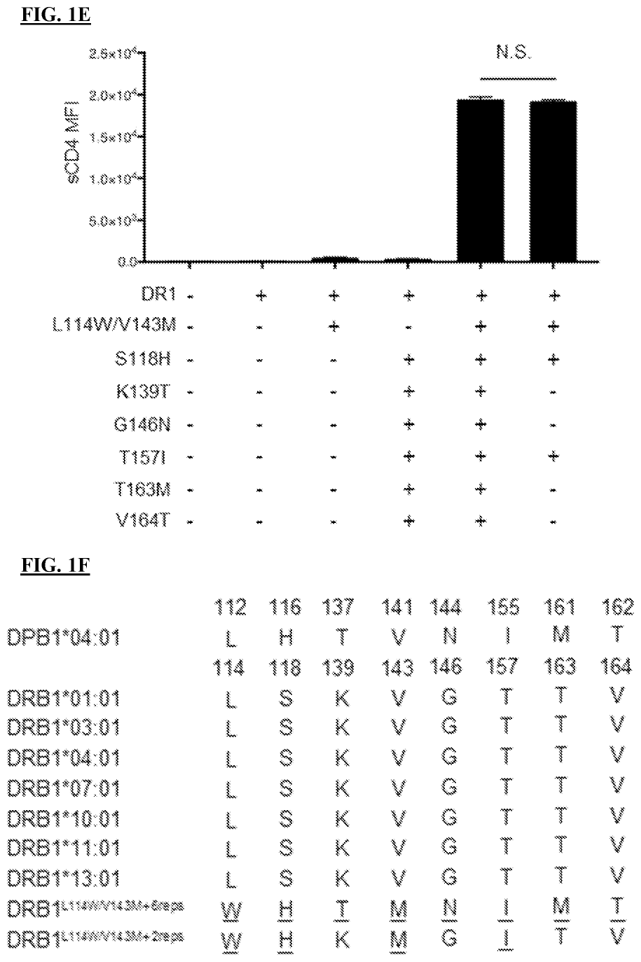

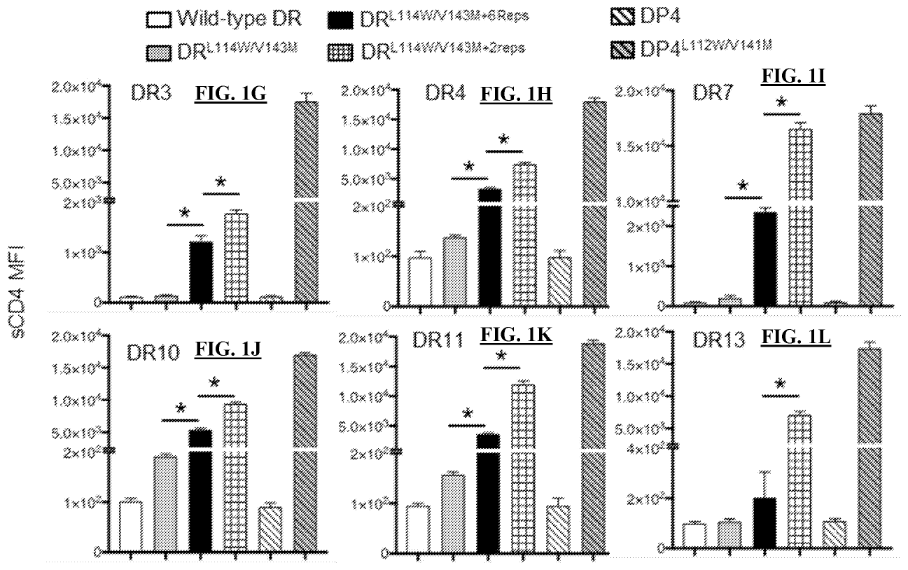

Affinity enhanced DR molecules were generated by introducing L114W/V143M mutations, to determine if these substitutions could improve the binding of HLA-DR molecules such as DR1 allele (DRA1*01:01-DRB1*01:01) to CD4. DRB1*01:01 encodes six different amino acids at positions 118, 139, 146, 157, 163 and 164 in addition to 114 and 143 (FIG. 1A). DR1^{L114W/V143M+6reps} showed enhanced CD4 binding compared with DR1^{L114W/V143M} and wild-type DR1 (FIGS. 1B and 1C). A library of DR1^{L114W/V143M+6reps}-derived mutants with a single amino acid reversal at either S118H or T157I but not at the 4 other positions showed decreased CD4 binding capability, suggesting that both the S118H and T157I mutations are critical (FIG. 1D).

Indeed, the CD4 binding capacity of DR1^{L114W/V143M+2reps}, which possesses the L114W/V143M+S118H/T157I replacements (2reps) in the (3 chain, was comparable to that of DR1^{L114W/V143M+6reps} (FIG. 1E). These results suggest that the two additional replacements at S118H and T157I are pivotal for the function of the L114W/V143M mutations in the improvement of the binding of DR1 to CD4.

DRβ chains such as DRB1*03:01, 04:01, 07:01, 10:01, 11:01, and 13:01 encode different amino acids at positions 118, 139, 146, 157, 163, and 164 in addition to 114 and 143 (FIG. 1F). Interestingly, comparison of CD4 binding activity between the DR1^{114W/V143M+2reps} and DR1^{L114W/V143M+6reps} mutants showed that, unlike for DR1, the L114W/V143M+2reps mutations enabled improved CD4 binding compared to the L114W/V143M+6reps mutations for DR3, DR4, DR7, DR10, DR11, and DR13 (FIGS. 1G-1L).

Using a biolayer interferometry (BLI) binding assay, the affinities of wild-type DR1 and DR1^{L114W/V143M+2reps} for CD4 were measured. While no binding was detected between wild-type DR1 and CD4 (FIG. 1M), DR1^{L114W/V143M+2reps} bound to CD4 with a KD of 14 μM±2.3 (FIGS. 1N-10).

### Example 3—Affinity-Matured DR Dimers Specifically and Robustly Stained Cognate TCRs

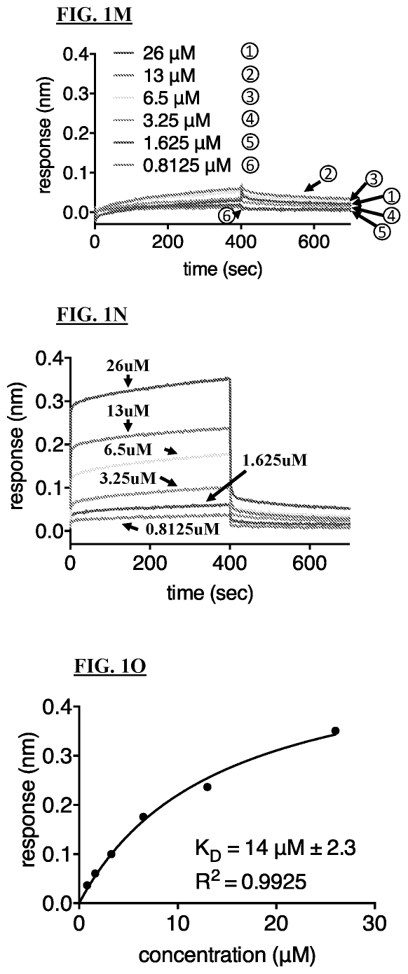

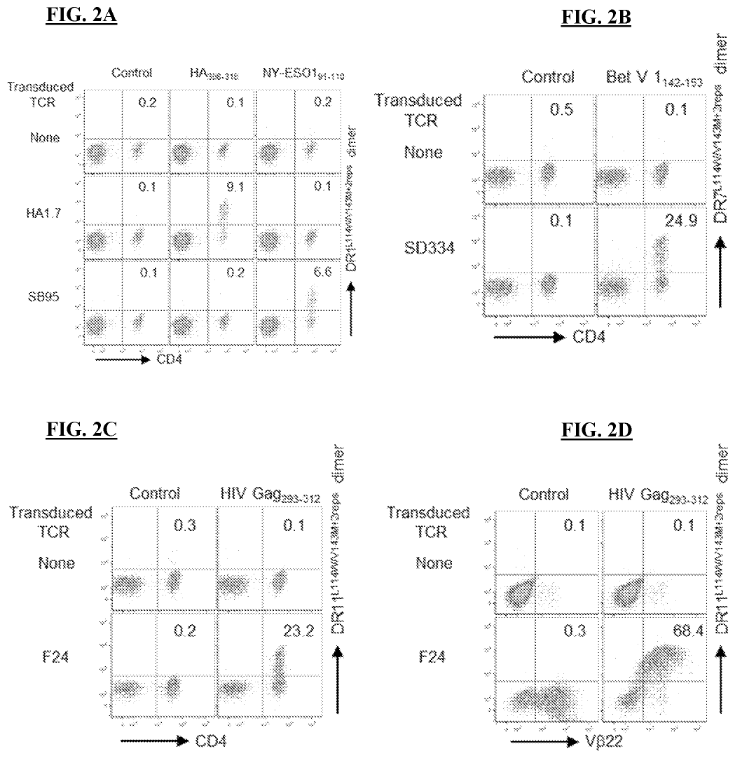

The ability of the affinity-matured DR dimers carrying the mutations described in example 2 were evaluated for the ability to identify antigen-specific CD4⁺ T cells. The DR1^{L114W/V143M+2reps}, DR7^{L114W/V143M+2reps}, and DR11^{L114W/V143M+2reps} dimers specifically stained the DR1-restricted TCRs HA1.7 and SB95, DR7-restricted TCR SD334, and DR11-restricted TCR F24, respectively (FIGS. 2A-2C). Costaining of F24-transduced CD4⁺ T cells with an anti-Vβ22 mAb, along with the respective DR11^{L114W/V143M+2reps} dimers, confirmed that virtually all the TCR-transduced CD4⁺ T cells were successfully stained with the respective DR11^{L114W/V143M+2reps} dimers (FIG. 2D).

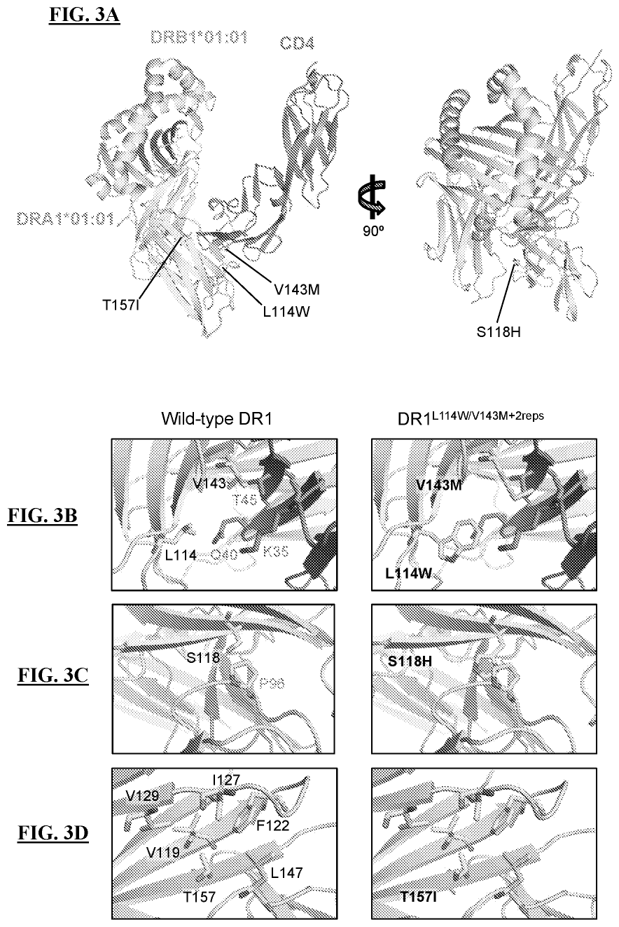

A structural model of the complex consisting of CD4 and DR1^{L114W/V143M+2reps} also showed a potential hydrophobic effect of the L114W/V143M replacements (FIGS. 3A-3B). Furthermore, hydrophobic stacking was observed between P96 of the α-chain and S118H of the β-chain (FIG. 3C), and the T157I replacement was found to localize in the β-sheet surrounding V119, F112, I127, V129, L147 and T157 (FIG. 3D). It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

To identify affinity-matured class II molecules, the present examples detail multiple mutations in the β-chain but not the α-chain because the β-chain has a more direct interaction with CD4 than the α chain. It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

In conclusion, CD4⁺ T cells play a critical role in the development of autoimmune diseases and protection against pathogenic infections and cancers. The novel HLA class II multimer technology described herein may better facilitate the study of HLA class II-restricted CD4⁺ T cell responses across HLA-DR alleles.

### Example 4

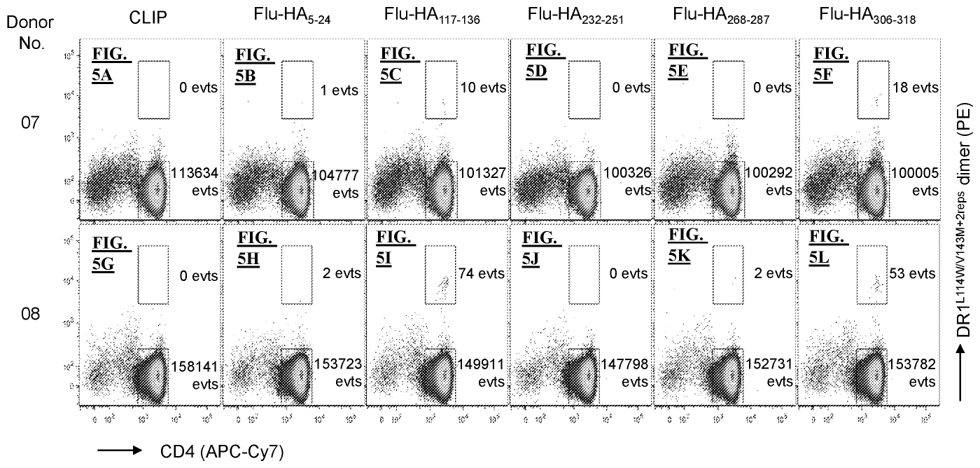

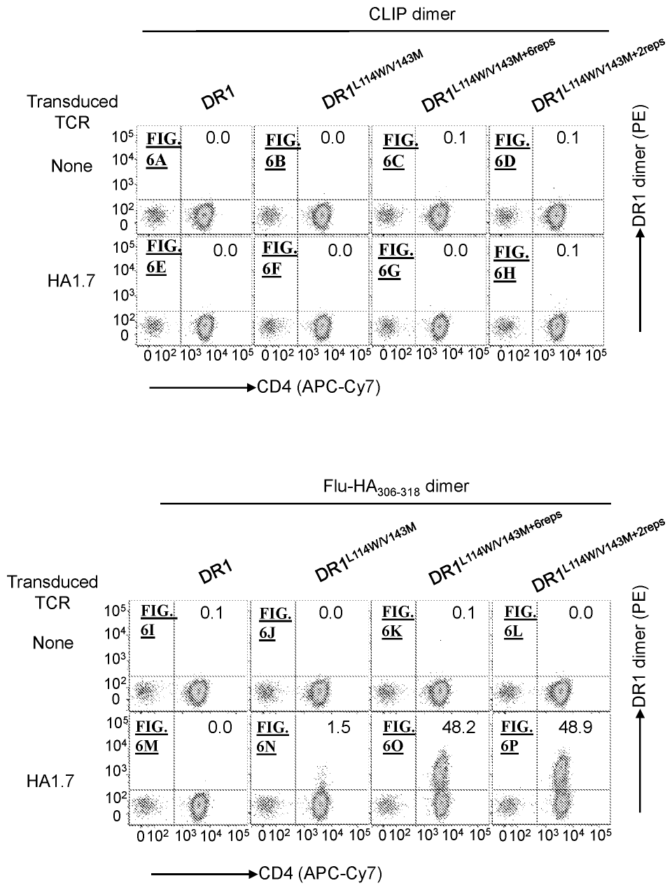

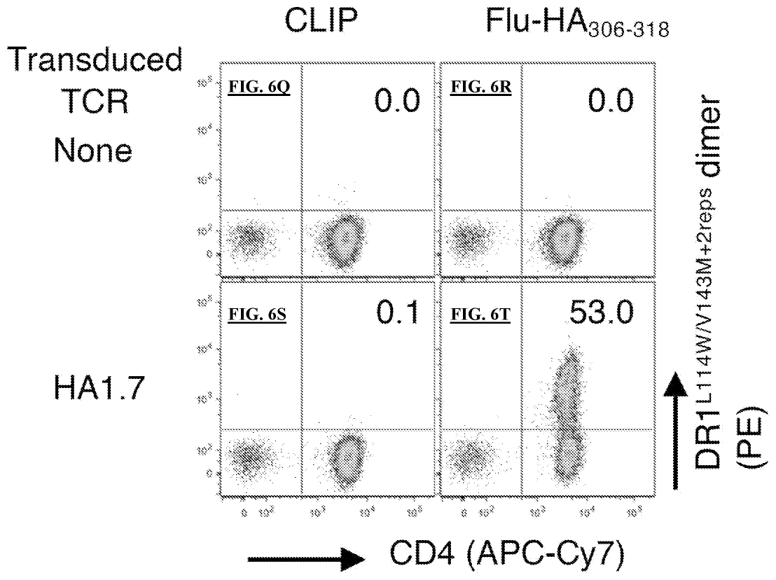

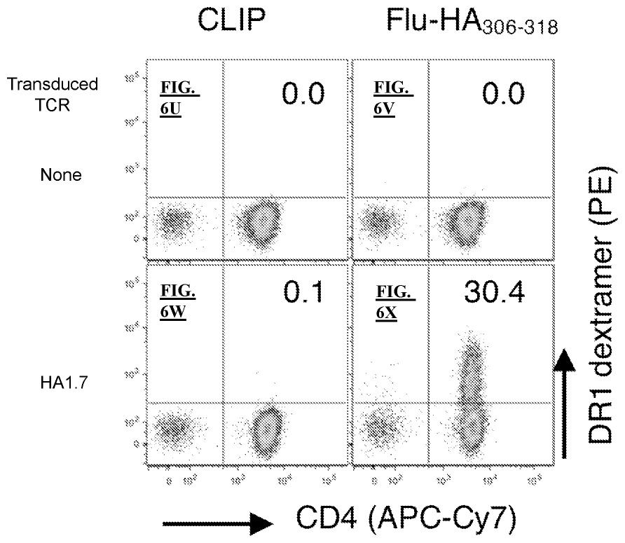

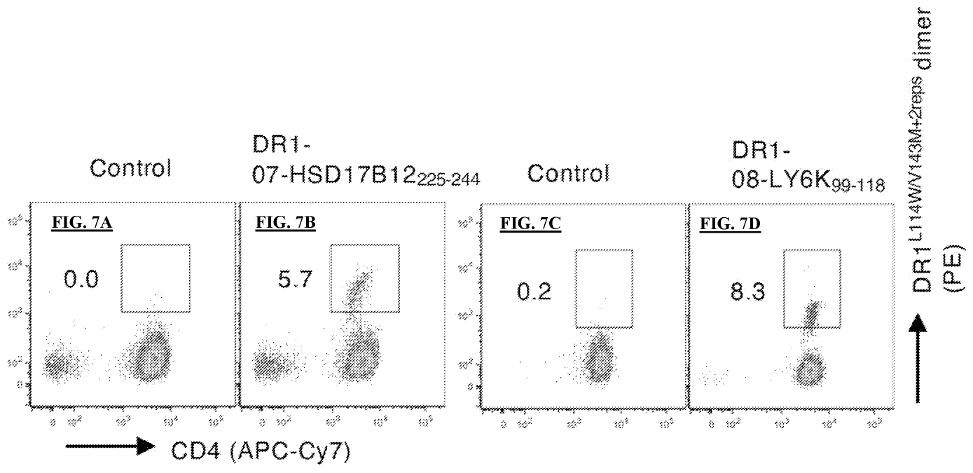

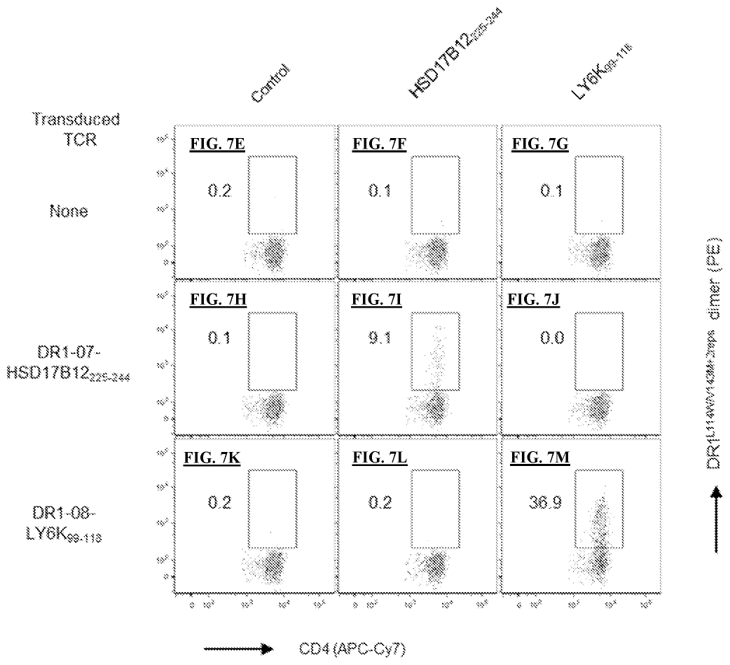

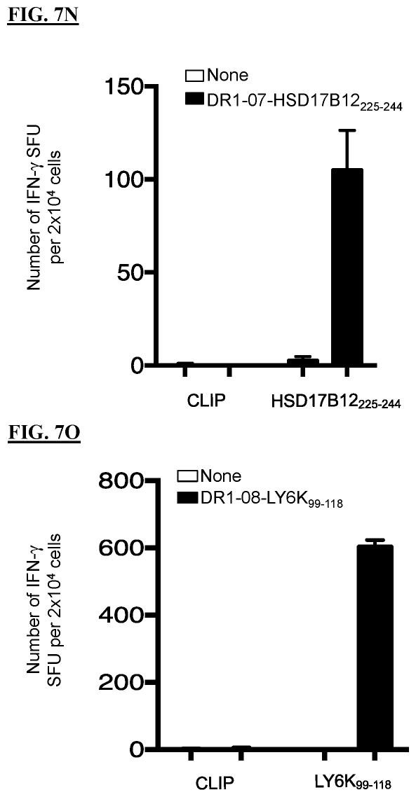

Ex vivo staining was performed of memory CD4⁺ T cells with DR1^{L114W/V143M+2reps} dimers specific to influenza virus hemagglutinin (Flu-HA) peptides without in vitro stimulation. A small subset of the CD4⁺ T cells were positively stained with DR1^{L114W/V143M+2reps} dimers for Flu-HA₁₁₇₋₁₃₆- and Flu-HA₃₀₆₋₃₁₈ (FIGS. 5A-5L). Wild-type DR1, DR1^{L114W/V143M} and DR1^{L114W/V143M+6reps} dimers and DR1^{L114W/V143M+2reps} dimers were produced and their staining of TCR-transduced CD4⁺ T cells was compared. Both wild-type DR1 and DR1^{L114W/V143M} dimers detected very little of the cognate TCR (HA1.7) on CD4⁺ T cells, while DR1^{L114W/V143M+2reps} and DR1^{L114W/V143M+6reps} dimers showed similar robust staining. Importantly, DR1^{L114W/V143M+2reps} dimers stained HA1.7-transduced CD4⁺ T cells more robustly and with better separation than the wild-type DR1 dextramer (FIGS. 6A-6X). To validate DR1^{L114W/V143M+2reps} dimer staining, DR1-restricted TCR genes specific to HSD17B12₂₂₅₋₂₄₄ and LY6K₉₉₋₁₁₈ were cloned from dimer⁺ CD4⁺ T cells in vitro expanded in a peptide-specific manner. When clonotypically reconstituted in primary CD4⁺ T cells, the two TCRs (Table 4) were successfully stained by the cognate DR1^{L114W/V143M+2reps} dimers and were functional in a DR1-restricted and antigen-specific manner (FIGS. 7A-7O).

TABLE 4
TCR Sequences

| | | | | | | | TCRβ |
|---|---|---|---|---|---|---|---|
| No. | Peptide | TRAV | TRAJ | TCRα CDR3 | TRBV | TRBJ | CDR3 |
| 07 | HSD17 | 5*01 | 4* | CADLSGG | 11* | 2-3* | CASSPTL |
| | B12₂₂₅₋₂₄₄ | | 01 | YNKLIF | 01 | 01 | GTDTQYF |
| | | | | (SEQ ID | | | (SEQ ID |
| | | | | NO: 22) | | | NO: 23) |
| 08 | LY6K₉₉₋₁₁₈ | 38- | 52* | CAYRSFL | 20- | 1-6* | CAASRES |
| | | 2/DV8* | 01 | NAGGTS | 1*01 | 01 | KWSSYN |
| | | 01 | | YGKLTF | | | SPLHF |
| | | | | (SEQ ID | | | (SEQ ID |
| | | | | NO: 24) | | | NO: 21) |

Methods

Cells

Peripheral mononuclear cells were obtained via density gradient centrifugation. K562-based artificial antigen presenting cells (aAPCs) individually expressing various HLA class II genes as a single HLA allele in conjunction with CD80 and CD83 have been reported previously (see Butler, M. O. et al., PLoS One 7, e30229 (2012)). The Jurkat 76 cell line is a T cell leukemic cell line lacking endogenous TCR, CD4, and CD8 expression (see Heemskerk, M. H. et al., Blood 102, 3530-3540 (2003)). Jurkat 76/CD4 cells were generated by retrovirally transducing the human CD4 gene. A375 cells are a melanoma cell line. HEK293T cells and A375 cells were grown in DMEM supplemented with 10% FBS and 50 μg/ml gentamicin. The Jurkat 76 cell line was cultured in RPMI 1640 supplemented with 10% FBS and 50 μg/ml gentamicin.

Peptides

Synthetic peptides were dissolved at 50 mg/ml in DMSO.

Genes

Novel TCR genes were cloned via 5′-rapid amplification of cDNA ends (RACE) PCR and sequenced as previously described (Nakatsugawa, M. et al., Sci Rep 6, 23821 (2016); Nakatsugawa, M. et al., J Immunol 194, 3487-3500 (2015); Ochi, T. et al., Cancer Immunol Res 3, 1070-1081 (2015)). All genes were cloned into the pMX retroviral vector and transduced into cell lines using the 293GPG and PG13 cell-based retrovirus system (see Hirano, N. et al., Blood 107, 1528-1536 (2006); Butler, M. O. et al., Clin Cancer Res 13, 1857-1867 (2007); Hirano, N. et al., Clin Cancer Res 12, 2967-2975 (2006)).

Antibodies

The following antibodies were used for flow cytometry analysis: APC-Cy7-conjugated anti-CD4 (RPA-T4; see Wooldridge, L. et al., Eur J Immunol 36, 1847-1855 (2006)) and PE-conjugated anti-His tag (AD1.1.10, ABCAM, Cambridge, Mass.). Dead cells were distinguished with the LIVE/DEAD Fixable Aqua Dead Cell Stain Kit. Stained cells were analyzed with FACSCanto II or LSRFortessa X-20. Cell sorting was conducted using a FACSAria II. Data analysis was performed using FlowJo software (version 9.9.6).

Generation of the HLA class II monomer and dimer HEK293T cells were transfected with the α and β genes using the 293GPG cell-based retrovirus system (see Hirano, N. et al., Blood 107, 1528-1536 (2006); Butler, M. O. et al., Clin Cancer Res 13, 1857-1867 (2007); Hirano, N. et al., Blood 108, 2662-2668 (2006)) and cultured in DMEM supplemented with 10% FBS and 50 μg/ml gentamicin. After forty-eight hours, the conditioned medium was harvested. The soluble HLA class II-containing supernatant was then mixed with 100 μg/ml peptide of interest for 20-24 hours at 37° C. for in vitro peptide exchange. The concentration of the monomer was measured by specific ELISA using a nickel-coated plate and an anti-His tag biotinylated mAb. Soluble HLA class II monomer was dimerized using PE-conjugated anti-His mAb (AD1.1.10) at a 2:1 molar ratio for 1.5 hours at 4° C. for staining.

Stimulation of DR1-Restricted Antigen-Specific CD4⁺ T Cells

CD4⁺ T cells were purified and then stimulated with DR1-expressing aAPCs pulsed with DR1-restricted peptides at 10 μg/ml and irradiated at 200 Gy at an E:T ratio of 20:1. After forty-eight hours, 10 IU/ml IL-2 and 10 ng/ml IL-15 were added to the CD4⁺ T cells. The culture medium supplemented with IL-2 (10 IU/ml) and IL-15 (10 ng/ml) was replenished every 2-3 days. Two weeks later, the T cells were subjected to DR1^{L114W/V143M+2reps} dimer staining.

HLA Class II Dimer and Dextramer Staining

DR1 dextramers were compared in multimer staining analysis. Primary CD4⁺ T cells transduced with antigen-specific TCR genes were pretreated with 50 nM dasatinib for 30 min at 37° C. and stained with 5-15 μg/ml class II dimers for 4-5 hours at room temperature. After washing, cell surface molecules were counterstained with an APC-Cy7-conjugated anti-CD4 mAb.

Dimer Staining of Unstimulated CD4⁺ T Cells from PBMCs from Melanoma Patients

One million CD4⁺ T cells were purified and pretreated with 50 nM dasatinib for 30 min at 37° C. The cells were stained with 5-15 μg/ml class II dimers for 4-5 hours at room temperature. After washing, cell surface molecules were counterstained with an APC-Cy7-conjugated anti-CD4 mAb. The absolute counts of the dimer⁺ cells were determined by flow cytometry.

ELISPOT Assay

Cytokine ELISPOT assays were performed as previously reported (see, e.g., Yamashita, Y. et al., Nat Commun 8, 15244 (2017); and Anczurowski, M. et al., Sci Rep 8, 4804 (2018)).

1. An HLA class II molecule comprising a DR beta chain, wherein the DR beta chain comprises an amino acid other than leucine at a position corresponding to amino acid residue 114 of SEQ ID NO: 1.

2. An HLA class II molecule comprising a DR beta chain, wherein the DR beta chain comprises a substitution mutation at a position corresponding to amino acid residue 114 of SEQ ID NO: 1, wherein the substitution mutation is with an amino acid other than leucine.

3. The HLA class II molecule of claim 1 or 2, wherein the DR beta chain further comprises an amino acid other than valine at a position corresponding to amino acid residue 143 of SEQ ID NO: 1.

4. An HLA class II molecule comprising a DR beta chain, wherein the DR beta chain comprises an amino acid other than valine at a position corresponding to amino acid residue 143 of SEQ ID NO: 1.

5. An HLA class II molecule comprising a DR beta chain, wherein the DR beta chain comprises a substitution mutation at a position corresponding to amino acid residue 143 of SEQ ID NO: 1, wherein the substitution mutation is with an amino acid other than valine.

6. The HLA class II molecule of claim 4 or 5, wherein the DR beta chain further comprises an amino acid other than leucine at a position corresponding to amino acid residue 114 of SEQ ID NO: 1.

7. The HLA class II molecule of any one of claims 1 to 6, wherein the DR beta chain further comprises an amino acid other than asparagine at a position corresponding to amino acid residue 110 of SEQ ID NO: 1.

8. The HLA class II molecule of any one of claims 1 to 7, wherein the DR beta chain further comprises an amino acid other than isoleucine at a position corresponding to amino acid residue 116 of SEQ ID NO: 1.

9. The HLA class II molecule of any one of claims 1 to 8, wherein the DR beta chain further comprises an amino acid other than serine at a position corresponding to amino acid residue 118 of SEQ ID NO: 1.

10. The HLA class II molecule of any one of claims 1 to 9, wherein the DR beta chain further comprises an amino acid other than proline at a position corresponding to amino acid residue 146 of SEQ ID NO: 1.

11. The HLA class II molecule of any one of claims 1 to 10, wherein the beta chain of the HLA class II molecule comprises a DR2, DR3, DR4, DR5, DR6, DR7, DR8, DR9, DR10, DR11, DR12, DR13, DR14, DR15, or DR16 allele.

12. The HLA class II molecule of any one of claims 1 to 10, wherein the beta chain of the MEW class II molecule comprises an HLA allele selected from the group consisting of DRB1*01, DRB1*03, DRB1*04, DRB1*07, DRB1*08, DRB1*09, DRB1*10, DRB1*11, DRB1*12, DRB1*13, DRB1*14, DRB1*15, and DRB1*16.

13. The HLA class II molecule of any one of claims 1 to 12, further comprising an alpha chain.

14. The HLA class II molecule claim 13, wherein the alpha chain of the MHC class II molecule comprises an HLA-DRA1*01 allele.

15. The HLA class II molecule of any one of claims 1 to 14, wherein the beta chain comprises:
(a) an amino acid other than leucine at a position corresponding to amino acid residue 114 of SEQ ID NO: 1;
(b) an amino acid other than valine at a position corresponding to amino acid residue 143 of SEQ ID NO: 1; and
(c) at least two of:
(i) an amino acid other than serine at a position corresponding to amino acid residue 118 of SEQ ID NO: 1,
(ii) an amino acid other than lysine at a position corresponding to amino acid residue 139 of SEQ ID NO: 1,
(iii) an amino acid other than glycine at a position corresponding to amino acid residue 146 of SEQ ID NO: 1,
(iv) an amino acid other than threonine at a position corresponding to amino acid residue 157 of SEQ ID NO: 1,
(v) an amino acid other than threonine at a position corresponding to amino acid residue 163 of SEQ ID NO: 1, and
(vi) an amino acid other than valine at a position corresponding to amino acid residue 164 of SEQ ID NO: 1.

16. The HLA class II molecule of claim 15, wherein the beta chain comprises:
(c) at least three of:
(i) an amino acid other than serine at a position corresponding to amino acid residue 118 of SEQ ID NO: 1,
(ii) an amino acid other than lysine at a position corresponding to amino acid residue 139 of SEQ ID NO: 1,
(iii) an amino acid other than glycine at a position corresponding to amino acid residue 146 of SEQ ID NO: 1,
(iv) an amino acid other than threonine at a position corresponding to amino acid residue 157 of SEQ ID NO: 1,
(v) an amino acid other than threonine at a position corresponding to amino acid residue 163 of SEQ ID NO: 1, and
(vi) an amino acid other than valine at a position corresponding to amino acid residue 164 of SEQ ID NO: 1.

17. The HLA class II molecule of claim 15 or 16, wherein the beta chain comprises:
(c) at least four of:
(i) an amino acid other than serine at a position corresponding to amino acid residue 118 of SEQ ID NO: 1,
(ii) an amino acid other than lysine at a position corresponding to amino acid residue 139 of SEQ ID NO: 1,
(iii) an amino acid other than glycine at a position corresponding to amino acid residue 146 of SEQ ID NO: 1,
(iv) an amino acid other than threonine at a position corresponding to amino acid residue 157 of SEQ ID NO: 1,
(v) an amino acid other than threonine at a position corresponding to amino acid residue 163 of SEQ ID NO: 1, and
(vi) an amino acid other than valine at a position corresponding to amino acid residue 164 of SEQ ID NO: 1.

18. The HLA class II molecule of any one of claims 1 to 15, wherein the beta chain comprises:
(a) an amino acid other than leucine at a position corresponding to amino acid residue 114 of SEQ ID NO: 1,
(b) an amino acid other than valine at a position corresponding to amino acid residue 143 of SEQ ID NO: 1,
(c) an amino acid other than serine at a position corresponding to amino acid residue 118 of SEQ ID NO: 1, and
(d) an amino acid other than threonine at a position corresponding to amino acid residue 157 of SEQ ID NO: 1.

19. The HLA class II molecule of any one of claims 1 to 18, wherein the beta chain comprises:
(a) an amino acid other than leucine at a position corresponding to amino acid residue 114 of SEQ ID NO: 1,
(b) an amino acid other than valine at a position corresponding to amino acid residue 143 of SEQ ID NO: 1,
(c) an amino acid other than serine at a position corresponding to amino acid residue 118 of SEQ ID NO: 1,
(d) an amino acid other than lysine at a position corresponding to amino acid residue 139 of SEQ ID NO: 1,
(e) an amino acid other than glycine at a position corresponding to amino acid residue 146 of SEQ ID NO: 1,
(f) an amino acid other than threonine at a position corresponding to amino acid residue 157 of SEQ ID NO: 1,
(g) an amino acid other than threonine at a position corresponding to amino acid residue 163 of SEQ ID NO: 1, and
(h) an amino acid other than valine at a position corresponding to amino acid residue 164 of SEQ ID NO: 1.

20. The HLA class II molecule of any one of claims 1 to 52, wherein the amino acid other than leucine at a position corresponding to amino acid residue 114 of SEQ ID NO: 1 comprises a hydrophobic side chain.

21. The HLA class II molecule of any one of claims 1 to 3 and 6 to 20, wherein the amino acid other than leucine at a position corresponding to amino acid residue 114 of SEQ ID NO: 1 is selected from the group consisting of an alanine, a valine, an isoleucine, a methionine, a phenylalanine, a tyrosine, and a tryptophan.

22. The HLA class II molecule of any one of claims 1 to 3 and 6 to 21, wherein the amino acid other than leucine at a position corresponding to amino acid residue 114 of SEQ ID NO: 1 is a tryptophan.

23. The HLA class II molecule of any one of claims 3 to 22, wherein the amino acid other than valine at a position corresponding to amino acid residue 143 of SEQ ID NO: 1 comprises a hydrophobic side chain.

24. The HLA class II molecule of any one of claims 3 to 23, wherein the amino acid other than valine at a position corresponding to amino acid residue 143 of SEQ ID NO: 1 is selected from an alanine, an isoleucine, a leucine, a methionine, a phenylalanine, a tyrosine, and a tryptophan.

25. The HLA class II molecule of any one of claims 3 to 24, wherein the amino acid other than valine at a position corresponding to amino acid residue 143 of SEQ ID NO: 1 is a methionine.

26. The HLA class II molecule of any one of claims 1 to 25, wherein the beta chain of the MHC class II molecule comprises an amino acid other than serine at a position corresponding to amino acid residue 118 of SEQ ID NO: 1.

27. The HLA class II molecule of any one of claims 15 to 26, wherein the amino acid other than serine at a position corresponding to amino acid residue 118 of SEQ ID NO: 1 is selected from an arginine, a histidine, and a lysine.

28. The HLA class II molecule of any one of claims 15 to 27, wherein the amino acid other than serine at a position corresponding to amino acid residue 118 of SEQ ID NO: 1 is a histidine.

29. The HLA class II molecule of any one of claims 1 to 28, wherein the beta chain of the MHC class II molecule comprises an amino acid other than lysine at a position corresponding to amino acid residue 139 of SEQ ID NO: 1.

30. The HLA class II molecule of any one of claims 15 to 29, wherein the amino acid other than lysine at a position corresponding to amino acid residue 139 of SEQ ID NO: 1 is selected from a serine, a threonine, and a glutamine.

31. The HLA class II molecule of any one of claims 15 to 30, wherein the amino acid other than lysine at a position corresponding to amino acid residue 139 of SEQ ID NO: 1 is a threonine.

32. The HLA class II molecule of any one of claims 1 to 31, wherein the beta chain of the MHC class II molecule comprises an amino acid other than glycine at a position corresponding to amino acid residue 146 of SEQ ID NO: 1.

33. The HLA class II molecule of any one of claims 15 to 32, wherein the amino acid other than glycine at a position corresponding to amino acid residue 146 of SEQ ID NO: 1 is selected from a serine, an asparagine, a threonine, and a glutamine.

34. The HLA class II molecule of any one of claims 15 to 33, wherein the amino acid other than glycine at a position corresponding to amino acid residue 146 of SEQ ID NO: 1 is a glutamine.

35. The HLA class II molecule of any one of claims 1 to 34, wherein the beta chain of the MHC class II molecule comprises an amino acid other than threonine at a position corresponding to amino acid residue 157 of SEQ ID NO: 1.

36. The HLA class II molecule of any one of claims 15 to 35, wherein the amino acid other than threonine at a position corresponding to amino acid residue 157 of SEQ ID NO: 1 is selected from an alanine, a valine, an isoleucine, a leucine, a methionine, a phenylalanine, a tyrosine, and a tryptophan.

37. The HLA class II molecule of any one of claims 15 to 36, wherein the amino acid other than threonine at a position corresponding to amino acid residue 157 of SEQ ID NO: 1 is an isoleucine.

38. The HLA class II molecule of any one of claims 1 to 37, wherein the beta chain of the MHC class II molecule comprises an amino acid other than threonine at a position corresponding to amino acid residue 163 of SEQ ID NO: 1.

39. The HLA class II molecule of any one of claims 15 to 38, wherein the amino acid other than threonine at a position corresponding to amino acid residue 163 of SEQ ID NO: 1 is selected from an alanine, a valine, an isoleucine, a leucine, a methionine, a phenylalanine, a tyrosine, and a tryptophan.

40. The HLA class II molecule of any one of claims 15 to 39, wherein the amino acid other than threonine at a position corresponding to amino acid residue 163 of SEQ ID NO: 1 is a methionine.

41. The HLA class II molecule of any one of claims 1 to 40, wherein the beta chain of the MHC class II molecule comprises an amino acid other than valine at a position corresponding to amino acid residue 164 of SEQ ID NO: 1.

42. The HLA class II molecule of any one of claims 15 to 41, wherein the amino acid other than valine at a position corresponding to amino acid residue 164 of SEQ ID NO: 1 is selected from a serine, an asparagine, a threonine, and a glutamine.

43. The HLA class II molecule of any one of claims 15 to 42, wherein the amino acid other than valine at a position corresponding to amino acid residue 164 of SEQ ID NO: 1 is a threonine.

44. The HLA class II molecule of any one of claims 1 to 43, wherein the beta chain comprises:
(a) a tryptophan at a position corresponding to amino acid residue 114 of SEQ ID NO: 1,
(b) a methionine at a position corresponding to amino acid residue 143 of SEQ ID NO: 1,
(c) a histidine at a position corresponding to amino acid residue 118 of SEQ ID NO: 1, and
(d) an isoleucine at a position corresponding to amino acid residue 157 of SEQ ID NO: 1.

45. The HLA class II molecule of any one of claims 1 to 44, wherein the DR beta chain comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 1, 3, 4, and 5.

46. The HLA class II molecule of any one of claims 1 to 44, wherein the beta chain comprises:
(a) a tryptophan at a position corresponding to amino acid residue 114 of SEQ ID NO: 1;
(b) a methionine at a position corresponding to amino acid residue 143 of SEQ ID NO: 1; and
(c) at least two of:
(i) a histidine at a position corresponding to amino acid residue 118 of SEQ ID NO: 1,
(ii) a threonine at a position corresponding to amino acid residue 139 of SEQ ID NO: 1,
(iii) a glutamine at a position corresponding to amino acid residue 146 of SEQ ID NO: 1,
(iv) an isoleucine at a position corresponding to amino acid residue 157 of SEQ ID NO: 1,
(v) a methionine at a position corresponding to amino acid residue 163 of SEQ ID NO: 1, and
(vi) a threonine at a position corresponding to amino acid residue 164 of SEQ ID NO: 1.

47. The HLA class II molecule of any one of claims 1 to 46, wherein the beta chain comprises:
(a) a tryptophan at a position corresponding to amino acid residue 114 of SEQ ID NO: 1;
(b) a methionine at a position corresponding to amino acid residue 143 of SEQ ID NO: 1; and
(c) at least three of:
(i) a histidine at a position corresponding to amino acid residue 118 of SEQ ID NO: 1,
(ii) a threonine at a position corresponding to amino acid residue 139 of SEQ ID NO: 1,
(iii) a glutamine at a position corresponding to amino acid residue 146 of SEQ ID NO: 1,
(iv) an isoleucine at a position corresponding to amino acid residue 157 of SEQ ID NO: 1,
(v) a methionine at a position corresponding to amino acid residue 163 of SEQ ID NO: 1, and
(vi) a threonine at a position corresponding to amino acid residue 164 of SEQ ID NO: 1.

48. The HLA class II molecule of any one of claims 1 to 47, wherein the beta chain comprises:
(a) a tryptophan at a position corresponding to amino acid residue 114 of SEQ ID NO: 1;
(b) a methionine at a position corresponding to amino acid residue 143 of SEQ ID NO: 1; and
(c) at least four of:
(i) a histidine at a position corresponding to amino acid residue 118 of SEQ ID NO: 1,
(ii) a threonine at a position corresponding to amino acid residue 139 of SEQ ID NO: 1,
(iii) a glutamine at a position corresponding to amino acid residue 146 of SEQ ID NO: 1,
(iv) an isoleucine at a position corresponding to amino acid residue 157 of SEQ ID NO: 1,
(v) a methionine at a position corresponding to amino acid residue 163 of SEQ ID NO: 1, and
(vi) a threonine at a position corresponding to amino acid residue 164 of SEQ ID NO: 1.

49. The HLA class II molecule of any one of claims 1 to 48, wherein the beta chain comprises:
(a) a tryptophan at a position corresponding to amino acid residue 114 of SEQ ID NO: 1,
(b) a methionine at a position corresponding to amino acid residue 143 of SEQ ID NO: 1,
(c) a histidine at a position corresponding to amino acid residue 118 of SEQ ID NO: 1,
(d) a threonine at a position corresponding to amino acid residue 139 of SEQ ID NO: 1,
(e) a glutamine at a position corresponding to amino acid residue 146 of SEQ ID NO: 1,
(f) an isoleucine at a position corresponding to amino acid residue 157 of SEQ ID NO: 1,
(g) a methionine at a position corresponding to amino acid residue 163 of SEQ ID NO: 1, and
(h) a threonine at a position corresponding to amino acid residue 164 of SEQ ID NO: 1.

50. The HLA class II molecule of any one of claims 1 to 49, wherein the DR beta chain comprises the amino acid sequence set forth in SEQ ID NO: 3.

51. The HLA class II molecule of any one of claims 1 to 50, wherein the DR beta chain comprises the amino acid sequence set forth in SEQ ID NO: 4.

52. The HLA class II molecule of any one of claims 1 to 51, wherein the beta chain of the HLA class II molecule comprises a DR1, DR3, DR4, DR7, DR8, DR9, DR10, DR11, DR12, DR13, DR14, DR15, or DR16 allele.

53. The HLA class II molecule of any one of claims 1 to 38, wherein the beta chain of the MHC class II molecule comprises an HLA-DRB1*01, HLA-DRB1*03, HLA-DRB1*04, HLA-DRB1*06, HLA-DRB1*07, HLA-DRB1*08, HLA-DRB1*09, HLA-DRB1*10, HLA-DRB1*11, HLA-DRB1*12, HLA-DRB1*13, HLA-DRB1*14, HLA-DRB1*15, or HLA-DRB1*16 allele.

54. The HLA class II molecule of any one of claim 41, wherein the alpha chain of the MHC class II molecule comprises an HLA-DRA1*01 allele.

55. The HLA class II molecule of any one of claims 13 to 54, wherein the DR alpha chain comprises an amino acid sequence having at least about 80%, at least about 85%, at least about 90%, at least about 95%, at least about 96%, at least about 97%, at least about 98%, or at least about 99% sequence identity to SEQ ID NO: 6 or 8.

56. The HLA class II molecule of any one of claims 13 to 55, wherein the DR alpha chain comprises the amino acid sequence set forth in SEQ ID NO: 6 or 8.

57. The HLA class II molecule of any one of claims 1 to 56, wherein the DR beta chain has an increased affinity for a CD4 protein as compared to a reference HLA class II molecule, wherein the reference HLA class II molecule comprises a DR beta chain comprising (i) a leucine at a position corresponding to amino acid residue 114 of SEQ ID NO: 1 and/or (ii) a valine at a position corresponding to amino acid residue 143 of SEQ ID NO: 1.

58. The HLA class II molecule of claim 57, wherein the increased affinity is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, or at least about 1000.

59. The HLA class II molecule of any one of claims 1 to 58, wherein the DR beta chain is bound to a membrane of a cell.

60. The HLA class II molecule of any one of claims 1 to 58, wherein the DR beta chain is not bound to a membrane of a cell.

61. The HLA class II molecule of any one of claims 1 to 58, wherein the DR beta chain comprises an extracellular domain of a full length DR alpha chain.

62. The HLA class II molecule of claim 61, wherein the DR beta chain does not comprise a transmembrane domain of a full length DR beta chain.

63. The HLA class II molecule of any one of claims 13 to 62, wherein the DR alpha chain is bound to a membrane of a cell.

64. The HLA class II molecule of any one of claims 13 to 62, wherein the DR alpha chain is not bound to a membrane of a cell.

65. The HLA class II molecule of any one of claims 13 to 62, wherein the DR alpha chain comprises an extracellular domain of a full length DR alpha chain.

66. The HLA class II molecule of claim 65, wherein the DR alpha chain does not comprise a transmembrane domain of a full length DR alpha chain.

67. The HLA class II molecule of any one of claims 1 to 66, wherein the DR beta chain is linked to or associated with an inert particle.

68. The HLA class II molecule of claim 67, wherein the inert particle is a bead.

69. The HLA class II molecule of claim 67, wherein the inert particle is a nanoparticle.

70. The HLA class II molecule of claim 69, wherein the nanoparticle is selected from a pegylated iron oxide, chitosan, dextrane, gelatin, alginate, liposome, starch, branched polymer, carbon-based carrier, polylactic acid, poly(cyano)acrylate, polyethyleinemine, block copolymer, ply caprolactone, SPIONS, USPIONS, Cd/Zn-selenide, or silica nanoparticle.

71. The HLA class II molecule of claim 69 or 70, wherein the nanoparticle is a pegylated iron oxide nanoparticle.

72. The HLA class II molecule of any one of claims 1 to 71, wherein the DR beta chain comprises a signal peptide.

73. The HLA class II molecule of any one of claims 13 to 72, wherein the DR alpha chain comprises a signal peptide.

74. The HLA class II molecule of claim 72 or 73, wherein the signal peptide comprises the amino acid sequence set forth in SEQ ID NO: 9.

75. A nucleic acid molecule encoding the DR beta chain of any one of claims 1 to 74.

76. The nucleic acid molecule of claim 75, further encoding the DR alpha chain of any of claims 13 to 74.

77. The nucleic acid molecule of claim 75 or 76, comprising a nucleotide sequence having at least about 70%, at least about 75%, at least about 80%, at least about 85%, at least about 90%, at least about 95%, at least about 96%, at least about 97%, at least about 98%, or at least about 99% sequence identity to SEQ ID NO: 2.

78. A vector comprising the nucleic acid molecule of any one of claims 75 to 77.

79. A cell comprising the HLA class II molecule of any one of claims 1 to 75, the nucleic acid molecule of any one of claims 76 to 77, or the vector of claim 78.

80. The cell of claim 79, which is a mammalian cell or an insect cell.

81. The cell of claim 79 or 80, which is selected from a K562 cell, T2, HEK293, HEK293T, A375, SK-MEL-28, Me275, COS, a fibroblast cell, a tumor cell, or any combination thereof.

82. The cell of any one of claims 79 to 81, which lacks endogenous MHC class II DR beta chain expression.

83. The cell of any one of claims 79 to 82, which lacks endogenous MHC class II DR alpha chain expression.

84. A method of identifying a T cell receptor capable of binding an epitope in an MHC class II complex, comprising pulsing the cell of any one of claims 79 to 83 with one or more peptide comprising the epitope, and stimulating one or more CD4⁺ T cell with the APC.

85. A method of treating a disease or condition in a subject in need thereof, comprising administering to the subject the MHC class II molecule of any one of claims 1 to 74.

86. The method of claim 85, wherein the disease or condition is cancer or an infection.

87. The method of claim 86, wherein the cancer is selected from the group consisting of melanoma, bone cancer, pancreatic cancer, skin cancer, cancer of the head or neck, uterine cancer, ovarian cancer, rectal cancer, stomach cancer, uterine cancer, lung cancer, Hodgkin's Disease, non-Hodgkin's lymphoma (NHL), cancer of the esophagus, cancer of the small intestine, cancer of the urethra, chronic or acute leukemia, acute myeloid leukemia, chronic myeloid leukemia, acute lymphoblastic leukemia (ALL) (including non T cell ALL), chronic lymphocytic leukemia (CLL), cancer of the bladder, cancer of the kidney or ureter, carcinoma of the renal pelvis, glioma, squamous cell cancer, and combinations of said cancers.

88. The method of claim 85 or 86, wherein the cancer is relapsed or refractory.

89. The method of any one of claims 85 to 88, wherein the cancer is locally advanced.

90. The method of any one of claims 85 to 89, wherein the cancer is advanced.

91. The method of any one of claims 85 to 90, wherein the cancer is metastatic

92. The HLA class II molecule of any one of claims 1 to 75, which binds CD4 with a K_{D} of less than about 20 μM.

93. The HLA class II molecule of any one of claims 1 to 75, which binds CD4 with a K_{D} of about 14 μM or less.

94. A complex comprising the HLA class II molecule of any one of claims 1 to 75, 92, and 93 and a peptide, wherein the peptide is selected from the group consisting of NY-ESO1₉₁₋₁₁₀, Bet V 1₁₄₂₋₁₅₃, HIV Gag₂₉₃₋₃₁₂, HA₃₀₆₋₃₁₈, Flu-HA₅₋₂₄, Flu-HA₁₁₇₋₁₃₆, Flu-HA₂₃₂₋₂₅₁, Flu-HA₂₆₈₋₂₈₇, Flu-HA₃₀₆₋₃₁₈, HSD17B12₂₂₅₋₂₄₄, LY6K₉₉₋₁₁₈, and any combination thereof.


SEQUENCE LISTING (24 sequences)

SEQ ID NO: 1 - protein, 198 aa, Artificial Sequence
  synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 2 - protein, 594 aa, Artificial Sequence
  synthetic construct
GGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACCTCCTGGTCTGCTCTGTGAGTGGTTTCTATCCAGGCAGCATTGAAGTCAGGTGGTTCCGGAACGGCCAGGAAGAGAAGGCTGGGGTGGTGTCCACAGGCCTGATCCAGAATGGAGATTGGACCTTCCAGACCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAG

SEQ ID NO: 3 - protein, 198 aa, Artificial Sequence
  synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSK

SEQ ID NO: 4 - protein, 267 aa, Artificial Sequence
  synthetic construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSKGGGGSLEIEAAFLERENTALETRVAELRQRVQRLRNRVSQYRTRYGPLGGGK

SEQ ID NO: 5 - protein, 266 aa, Artificial Sequence
  synthetic construct
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 6 - protein, 192 aa, Artificial Sequence
  synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEN

SEQ ID NO: 7 - protein, 576 aa, Artificial Sequence
  synthetic construct
ATCAAAGAAGAACATGTGATCATCCAGGCCGAGTTCTATCTGAATCCTGACCAATCAGGCGAGTTTATGTTTGACTTTGATGGTGATGAGATTTTCCATGTGGATATGGCAAAGAAGGAGACGGTCTGGCGGCTTGAAGAATTTGGACGATTTGCCAGCTTTGAGGCTCAAGGTGCATTGGCCAACATAGCTGTGGACAAAGCCAACCTGGAAATCATGACAAAGCGCTCCAACTATACTCCGATCACCAATGTACCTCCAGAGGTAACTGTGCTCACAAACAGCCCTGTGGAACTGAGAGAGCCCAACGTCCTCATCTGTTTCATAGACAAGTTCACCCCACCAGTGGTCAATGTCACGTGGCTTCGAAATGGAAAACCTGTCACCACAGGAGTGTCAGAGACAGTCTTCCTGCCCAGGGAAGACCACCTTTTCCGCAAGTTCCACTATCTCCCCTTCCTGCCCTCAACTGAGGACGTTTACGACTGCAGGGTGGAGCACTGGGGCTTGGATGAGCCTCTTCTCAAGCACTGGGAGTTTGATGCTCCAAGCCCTCTCCCAGAGACTACAGAGAAC

SEQ ID NO: 8 - protein, 269 aa, Artificial Sequence
  synthetic construct
MMRPIVLVLLFATSALAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENGGGGSLEIRAAFLRQRNTALRTEVAELEQEVQRLENEVSQYETRYGPLGGGKGSHHHHHH

SEQ ID NO: 9 - protein, 17 aa, Artificial Sequence
  synthetic construct
MMRPIVLVLLFATSALA

SEQ ID NO: 10 - protein, 458 aa, Artificial Sequence
  synthetic construct
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 11 - protein, 198 aa, Artificial Sequence
  synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCSVSGFYPGSIEVRWFRNGQEEKAGVMSTGLIQNGDWTFQTLVMLETVPRSGEVYTCQVEHPSVTSPLTVEWRARSESAQSK

SEQ ID NO: 12 - protein, 198 aa, Artificial Sequence
  synthetic construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEETAGVMSTNLIQNGDWTFQILVMLEMTPRSGEVYTCQVEHPSVTSPLTVEWRARSESAQSK

SEQ ID NO: 13 - protein, 15 aa, Artificial Sequence
  synthetic construct
GLNDIFEAQKIEWHE

SEQ ID NO: 14 - protein, 267 aa, Artificial Sequence
  synthetic construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCSVSGFYPGSIEVRWFRNGQEEKAGVMSTGLIQNGDWTFQTLVMLETVPRSGEVYTCQVEHPSVTSPLTVEWRARSESAQSKGGGGSLEIEAAFLERENTALETRVAELRQRVQRLRNRVSQYRTRYGPLGGGK

SEQ ID NO: 15 - protein, 284 aa, Artificial Sequence
  synthetic construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSLEIEAAFLERENTALETRVAELRQRVQRLRNRVSQYRTRYGPLGGGKGSGLNDIFEAQKIEWHE

SEQ ID NO: 16 - protein, 855 aa, Artificial Sequence
  synthetic construct
ATGATGCGGCCCATCGTGCTGGTGCTGCTGTTCGCCACATCTGCCCTGGCCGGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACCTCCTGGTCTGCTCTGTGAGTGGTTTCTATCCAGGCAGCATTGAAGTCAGGTGGTTCCGGAACGGCCAGGAAGAGAAGGCTGGGGTGGTGTCCACAGGCCTGATCCAGAATGGAGATTGGACCTTCCAGACCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAGGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 17 - protein, 284 aa, Artificial Sequence
  synthetic construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSKGGGGSLEIEAAFLERENTALETRVAELRQRVQRLRNRVSQYRTRYGPLGGGKGSGLNDIFEAQKIEWHE

SEQ ID NO: 18 - protein, 855 aa, Artificial Sequence
  synthetic construct
ATGATGCGGCCCATCGTGCTGGTGCTGCTGTTCGCCACATCTGCCCTGGCCGGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACTGGCTGGTCTGCCATGTGAGTGGTTTCTATCCAGGCAGCATTGAAGTCAGGTGGTTCCGGAACGGCCAGGAAGAGAAGGCTGGGGTGATGTCCACAGGCCTGATCCAGAATGGAGATTGGACCTTCCAGATCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAGGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 19 - protein, 273 aa, Artificial Sequence
  synthetic construct
MMRPIVLVLLFATSALAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENGGGGSLEIRAAFLRQRNTALRTEVAELEQEVQRLENEVSQYETRYGPLGGGKGSHHHHHHHHHH

SEQ ID NO: 20 - protein, 822 aa, Artificial Sequence
  synthetic construct
ATGATGCGGCCCATCGTGCTGGTGCTGCTGTTCGCCACATCTGCCCTGGCCATCAAAGAAGAACATGTGATCATCCAGGCCGAGTTCTATCTGAATCCTGACCAATCAGGCGAGTTTATGTTTGACTTTGATGGTGATGAGATTTTCCATGTGGATATGGCAAAGAAGGAGACGGTCTGGCGGCTTGAAGAATTTGGACGATTTGCCAGCTTTGAGGCTCAAGGTGCATTGGCCAACATAGCTGTGGACAAAGCCAACCTGGAAATCATGACAAAGCGCTCCAACTATACTCCGATCACCAATGTACCTCCAGAGGTAACTGTGCTCACGAACAGCCCTGTGGAACTGAGAGAGCCCAACGTCCTCATCTGTTTCATCGACAAGTTCACCCCACCAGTGGTCAATGTCACGTGGCTTCGAAATGGAAAACCTGTCACCACAGGAGTGTCAGAGACAGTCTTCCTGCCCAGGGAAGACCACCTTTTCCGCAAGTTCCACTATCTCCCCTTCCTGCCCTCAACTGAGGACGTTTACGACTGCAGGGTGGAGCACTGGGGCTTGGATGAGCCTCTTCTCAAGCACTGGGAGTTTGATGCTCCAAGCCCTCTCCCAGAGACTACAGAGAACGGCGGCGGAGGCAGCCTGGAAATCAGAGCCGCCTTCCTGCGGCAGAGAAACACCGCCCTGAGAACCGAAGTGGCCGAGCTGGAACAGGAAGTGCAGCGGCTGGAAAACGAGGTGTCCCAGTACGAGACAAGATACGGCCCTCTGGGAGGCGGCAAGGGCTCTCACCACCACCATCACCATCATCATCACCATTGA

SEQ ID NO: 21 - protein, 18 aa, Artificial Sequence
  synthetic construct
CAASRESKWSSYNSPLHF

SEQ ID NO: 22 - protein, 13 aa, Artificial Sequence
  synthetic construct
CADLSGGYNKLIF

SEQ ID NO: 23 - protein, 14 aa, Artificial Sequence
  synthetic construct
CASSPTLGTDTQYF

SEQ ID NO: 24 - protein, 19 aa, Artificial Sequence
  synthetic construct
CAYRSFLNAGGTSYGKLTF